(19) United States
(12) Patent Application Publication
(10) Pub. No.: US 2026/0345773 A1
(43) Pub. Date: Nov. 5, 2026

(54) COMPOSITIONS AND METHODS FOR TREATING CANCER

(71) Applicant: The University of North Carolina at Chapel Hill, Chapel Hill, NC (US)

(72) Inventors: Yuliya Pylayeva-Gupta, Chapel Hill, NC (US); Bhalchandra Mirlekar, Memphis, TN (US)

(21) Appl. No.: 19/107,195

(22) PCT Filed: Sep. 1, 2023
(86) PCT No.: PCT/US2023/073316

§ 371 Date: Feb. 27, 2025

Related U.S. Application Data
(60) Provisional application No. 63/374,506, filed on Sep. 2, 2022.
(60) Provisional application No. 63/375,289, filed on Sep. 12, 2022.

(51) Int. Cl. A61K 40/13; A61K 40/42; C12N 5/0781

(52) U.S. Cl. CPC A61K 40/13; A61K 40/42; C12N 5/0635; C12N 2501/2335; C12N 2506/11

(57) ABSTRACT

This invention relates to methods of producing reprogrammed B cells, methods of treating cancer, and methods of enhancing an immune response in a subject having, suspected to have and/or at risk of cancer. The invention further relates to compositions and kits comprising reprogrammed B cells of the invention as well as use thereof in the methods of the invention.

### STATEMENT OF PRIORITY

This application claims the benefit, under 35 U.S.C. § 119(e), of U.S. Provisional Application Ser. No. 63/374,506, filed Sep. 2, 2022, and U.S. Provisional Application Ser. No. 63/375,289, filed Sep. 12, 2022, the entire contents of each of which are incorporated by reference herein.

### STATEMENT OF GOVERNMENT SUPPORT

This invention was made with government support under Grant Number CA230786 awarded by the National Institutes of Health. The government has certain rights in the invention.

### STATEMENT REGARDING ELECTRONIC FILING OF A SEQUENCE LISTING

A Sequence Listing in XML format, entitled 5470-937WO_ST26.xml, 90,989 bytes in size, generated on Sep. 1, 2023 and filed herewith, is hereby incorporated by reference into the specification for its disclosures.

### FIELD OF THE INVENTION

This invention relates to methods of producing reprogrammed B cells, methods of treating cancer, and methods of enhancing an immune response in a subject having, suspected to have and/or at risk of cancer. The invention further relates to compositions and kits comprising reprogrammed B cells of the invention as well as use thereof in the methods of the invention.

### BACKGROUND OF THE INVENTION

Plasma cell responses are associated with anti-tumor immunity and favorable response to immunotherapy. In theory, B cells can amplify anti-tumor immune responses through antibody production, yet B cells in patients and tumor-bearing mice often fail to support these effector function. For example, pancreatic ductal adenocarcinoma (PDAC) is an aggressive and deadly disease characterized by rampant immunosuppression and resistance to immunotherapy, where simple evaluation of B cell infiltration cannot distinguish between positive, neutral or negative patient prognoses (Clark et al. 2007 Cancer Research 67:9518-9527; Royal et al. 2010 J. of Immunotherapy 33:828; Brahmer et al. 2012 NEJM 366:2455-2465; Vonderheide and Bayne 2013 Current Opinion in Immunology 25:200-205; Feig et al. 2012 Clinical Cancer Research 18:4266-4276; Wouters and Nelson 2018 Clinical Cancer Research 24:6125-6135).

B cells frequently infiltrate human tumors, and the intra-tumoral abundance of plasma cells can correlate with improved patient prognosis. However, some tumors are devoid of plasma B cells, and strategies to enhance anti-tumor B cell responses are needed (Largeot et al. 2019 Cells 8:449). The transcriptional and signaling mechanisms that regulate B cell differentiation in malignancy are also not well understood, and there is a significant knowledge gap in the understanding of how effective anti-tumor B cell responses versus regulatory responses are generated. There is a need to develop new therapeutic methods for the management of overcoming immunosuppression in cancers.

### SUMMARY OF THE INVENTION

One aspect of the present invention provides a method of producing a reprogrammed B cell, comprising culturing a B cell (e.g., ex vivo culturing) in a medium with a reprogramming factor to produce a reprogrammed B cell.

Another aspect of the present invention provides a method of producing a population of reprogrammed B cells, comprising culturing a B cell (e.g., ex vivo culturing) in a medium with a reprogramming factor to produce a population of one or more reprogrammed B cells.

Another aspect of the present invention provides a method of treating a cancer in a subject, comprising delivering to the subject an effective amount of a reprogrammed B cell and/or B cell population of the invention.

Another aspect of the present invention provides a method of enhancing an immune response (e.g., a B and/or T cell response) to cancer in a subject, comprising delivering to the subject an effective amount of a reprogrammed B cell and/or B cell population of the invention.

Another aspect of the present invention provides a method of treating a cancer in a subject, comprising the steps of: (a) culturing a B cell (e.g., ex vivo culturing) in a medium with a reprogramming factor to produce a population of one or more reprogrammed B cells; and (b) delivering the reprogrammed B cells to the subject, thereby treating the cancer in the subject.

Another aspect of the present invention provides a method of enhancing an immune response (e.g., a B and/or T cell response) to cancer in a subject, comprising the steps of: (a) culturing a B cell (e.g., ex vivo culturing) in a medium with a reprogramming factor to produce a population of one or more reprogrammed B cells; and (b) delivering the reprogrammed B cells to the subject, thereby the immune response to the cancer in the subject.

Also provided are compositions and kits comprising a reprogrammed B cell produced by the methods of the invention and/or for use in the methods of the invention.

These and other aspects of the invention are set forth in more detail in the description of the invention below.

### BRIEF DESCRIPTION OF THE DRAWINGS

FIG. 1 shows fluorescence images and data plots related to studies performed examining increased accumulation of B cell subsets in human PDAC. FIG. 1 panel A) Representative immunofluorescence staining for CD20, CD138 and Ebi3 in samples of normal adjacent (left panel) and human PDAC. CD20+Ebi3+ B cells are indicated by arrowheads. Scale bars, 50 μm. FIG. 1 panel B) Quantification of the frequency of CD20+ cells in human PDAC (N=56) and adjacent normal tissues (N=23). FIG. 1 panel C) Quantification of the frequency of CD138+ cells in human PDAC and adjacent normal tissues. FIG. 1 panel D) Quantification of the frequency of CD20+Ebi3+ cells in human PDAC and adjacent normal tissues. FIG. 1 panel E) Paired analysis of intratumoral CD20+Ebi3+ immunoregulatory B cells and CD138+ plasma cells. FIG. 1 panel F) Association with survival via Cox PH regression analysis of plasma cell signature in TCGA PAAD dataset. FIG. 1 panel G) Correlation of cancer Breg signature with plasma cell signature in PAAD (TCGA). For IF quantification, three fields per sample were counted. Error bars indicate SEM, p− values were calculated using Student's t-test (unpaired, two-tailed); NS—not significant, p value: **<0.01; ***<0.001.

FIG. 2 shows data plots related to studied performed examining transcriptional profiling to identify disease-specific naïve B cell states. FIG. 2 panel A) Volcano plot of differentially expressed genes (red; upregulated, blue; downregulated) with p-adj<0.05 in naive B cells from (S3A). FIG. 2 panel B) Bar plots of the top enriched GO biological processes in healthy and tumor-educated naïve B cells. FIG. 2 panel C) Bar plots of the top enriched GO biological processes in healthy and tumor-educated naïve BEbi3−/− cells. FIG. 2 pane D) Loading plot overlayed on Principle Component Analysis (PCA) as derived by DEG analysis between healthy wild-type (WT), healthy B cell-specific Ebi3 KO (BEbi3−/−), tumor-educated wild-type (WT_tum) and tumor-educated B cell-specific Ebi3 KO (BEbi3−/− tum) naïve B cells. FIG. 2 panel E) Heatmap of selected differentially expressed genes in naïve B cells from healthy or tumor-bearing WT and BEBi3−/− mice. The standardization of log expression was performed by row. FIG. 2 panel F) Quantification of intracellular pSTAT3 and Pax5 as determined by flow cytometry in splenic Breg cells isolated from WT mice and activated with αCD40/LPS (Sti, blue) and αCD40/LPS+IL35 (red) for 48 hours. FIG. 2 panel G) Quantification of intracellular pSTAT3 (left), Pax5 (middle) and BCL6 (right) analyzed by flow cytometry from intra-tumoral Bcon and Breg. FIG. 2 panel H) Relative levels of indicated gene expression in each group, as determined by RNAseq of peripheral CD19+CD24hiCD38 hi Breg or CD19+CD24loCD38lo Bcon cells from PDAC patients. FIG. 2 panel I) Quantification of IL35 expression from Breg and Bcon cells isolated from spleens of PDAC patients (n=25).

FIG. 2 panel J) The MFI of the intracellular levels of pSTAT3 (left) and Pax5 (right) in Breg and Bcon cells isolated as in FIG. 2 panel H. Data are representative of 3 independent experiments. Error bars indicate SEM. NS: non-significant, *p<0.05, **p<0.005 (Student t test, two-tailed, unpaired).

FIG. 3 shows data plots related to studies performed indicating that IL35 drives imbalance of plasma and regulatory B cells in cancer. FIG. 3 panel A) Quantification of tumor weights from WT, BEBi3−/− and Bp35−/− mice orthotopically injected with KPC cells and collected at 3 weeks post-injection. Control WT mice represent both BEBi3+/− and BWT control bone marrow chimera groups. FIG. 3 panels B and C) Frequency of intra-tumoral naive B cells (% of CD45; panel B), and CD21hiCD1dhiCD5+ Breg cells (% of CD19; panel C) and total B cells (% of CD45; panel C) from FIG. 3 panel A as determined by flow cytometry. FIG. 3 panels D and E) Representative flow cytometry plots (panel D) and quantification (panel E) of frequency of IL-10, and IL35 expressing Breg cells from FIG. 3 panel A. FIG. 3 panels F and G) Representative flow cytometry plots (panel F) and quantification (panel G) of CD19loCD138 hi intra-tumoral plasma cells from FIG. 3 panel A. FIG. 3 panels H and I) Quantification of IgG (panel H) and IgM levels (panel I) in tumors of WT, BEBi3−/− and Bp35−/− mice 3− week post-orthotopic injection with KPC cells. FIG. 3 panel J) Representative ADCC assay for determination of antibody mediated killing of tumor cells (target cells). Target 1: Non-cancerous pancreatic cells, Target 2: Tumor cells (KPC 4662), ADCC: Effector cells+Target cells+source of antibodies (serum samples) from tumor bearing WT, BEBi3−/− and Bp35−/− mice. RLU: Relative Luminescence Units. FIG. 3 panel K) Schematic of treatment schedule. Representative flow cytometry plots of intratumoral CD19loCD138 hi plasma cells from Bortezomib (BZ) or control PBS treated WT and BEBi3−/− mice 3-week post-orthotopic injection with KPC cells. FIG. 3 panel L) Quantification of intra-tumoral CD19loCD138 hi plasma cells from FIG. 3 panel K. FIG. 3 panel M) Quantification of pancreatic tumor weights from FIG. 3 panel K. Error bars indicate SEM; p values in FIG. 3 panels A-I were calculated using one-way ANOVA. P values in FIG. 3 panel J were calculated using student t test, two-tailed, unpaired. p values in FIG. 3 panels L and M were calculated using two-way ANOVA. NS: non-significant, *p<0.05, **p<0.005, ***p<0.001. Data are representative of three independent experiments.

FIG. 4 shows bar graphs related to studies performed examining how tumor-educated naïve B cells are primed for dysfunction. FIG. 4 panel A) Intracellular levels of EBi3 and p35 in naive B cells from WT and orthotopic KPC (oKPC) mice treated with LPS/αCD40±indicated cytokines for 72 hr as determined by flow cytometry. FIG. 4 panel B) Intracellular levels of Pax5 in naive B cells from WT and oKPC mice treated as in FIG. 4 panel A were determined by flow cytometry. FIG. 4 panel C) Intracellular levels of Pax5 and BCL6 in LPS/αCD40/rIL35 treated naïve B cells isolated from WT and oKPC mice as determined by flow cytometry. FIG. 4 panels D-G) Expression of Ebi3 and p35 (FIG. 4 panel D), CD1d and IL-10 (FIG. 4 panel E), Pax5 and Bcl6 (FIG. 4 panel F), and pSTAT1 and pSTAT3 (FIG. 4 panel G) in naive B cells from WT and spontaneous KPC (sKPC) mice treated as in FIG. 4 panel A) were determined by flow cytometry. FIG. 4 panels H-J) Fold change in expression of P35, EBI3, and IL10 (FIG. 4 panel H); BCL6 and PAX5 (FIG. 4 panel I); and PRDM1 and XBP1 (FIG. 4 panel J) from naïve B cells isolated from spleens of PDAC patients and activated with αCD40/LPS±rIL35. FIG. 4 panel K) Heat map of top 30 differentially expressed chromatin and DNA modifiers, fold expression is indicated. FIG. 4 panels L-N) Relative H3K27 methylation (FIG. 4 panel L); HDAC1 acetylation (FIG. 4 panel M); and H3K27 acetylation (FIG. 4 panel N) enrichment as determined by ChIP assay at p35, EBi3 and Cd1d gene promoters. Naïve B cells isolated from WT and oKPC mice were treated with αCD40/LPS and rIL35 for 72 hrs then used for downstream ChIP analysis. FIG. 4 panels O and P) Relative H3K27 acetylation enrichment as determined by ChIP assay at EBI3 (FIG. 4 panel O) and P35 (FIG. 4 panel P) gene promoters from Breg and Bcon cells isolated from spleens of PDAC patients. Data are representative of 3 independent experiments. Error bars indicate SEM. NS: non-significant, *p<0.05, **p<0.01, ***p<0.005 (Student t test, two-tailed, unpaired).

FIG. 5 shows bar graphs and data plots related to studies performed examining how IL35 drives STAT3-Pax5 co-recruitment in tumor-educated B cells. FIG. 5 panel A) Mouse Pax5 gene promoter with the STAT binding consensus sites. FIG. 5 panel B) Mouse Bcl6 gene promoter with the STAT and Pax5 binding consensus sites. FIG. 5 panel C) ChIP analysis for enrichment of pSTAT1 and pSTAT3 on Pax5 promoter in naïve B cells (treated with αCD40/LPS/rIL35 for 72 hr) from WT or orthotopic KPC (oKPC) mice. FIG. 5 panels D and E) ChIP analysis for enrichment of pSTAT3 (FIG. 5 panel D) and Pax5 (FIG. 5 panel E) at Bcl6 promoter from naïve B cells isolated and treated as in FIG. 5 panel C. FIG. 5 panel F) Representative flow cytometry plot of Duolink proximity ligation assay (PLA) between pSTAT3 and Pax5 in naïve B cells treated with LPS/αCD40/cytokine (green) or LPS/αCD40 alone. FIG. 5 panel G) Representative flow cytometry plot of PLA between pSTAT3 and Pax5 in intra-tumoral Breg (green) and Bcon (black) cells. FIG. 5 panel H) ChIP-re-ChIP analysis to detect STAT3-Pax5 complex (STAT3:S3, Pax5:P5) at Bcl6 promoter in rIL35 treated naïve B cells isolated from WT and orthotopic KPC (oKPC) mice. FIG. 5 panels I-K) ChIP-re-ChIP analysis to detect STAT3-Pax5 complex (STAT3:S3, Pax5:P5) at promoters of p35 (FIG. 5 panel I), EBi3 (FIG. 5 panel J) and Cd1d (FIG. 5 panel K) genes in rIL35 treated naïve B cells isolated from WT or oKPC mice. Error bars indicate SEM; p values were calculated using two-tailed; unpaired, Student t-test. NS: non-significant, *p<0.05, **p<0.005. Data represent triplicates within same experiment and are representative of three independent experiments.

FIG. 6 shows data plots and bar graphs related to studies performed examining how the IL35-STAT3 axis deregulates naive B cell in pancreatic cancer. FIG. 6 panel A) Representative flow cytometry histogram plot of intracellular IL10, p35, EBi3 and surface CD1d in naïve B cells isolated from orthotopic KPC mice and treated as indicated. FIG. 6 panel B) Quantification of IL10, p35, EBi3 and CD1d in naive B cells treated as in FIG. 6 panel A, with STAT1 inhibitor (left) and STAT3 inhibitor (right). FIG. 6 panel C) Quantification of Pax5 in naive B cells treated as in FIG. 6 panel A. FIG. 6 panel D) Quantification of Bcl6 in naive B cells treated as in FIG. 6 panel A. FIG. 6 panel E) ChIP analysis for enrichment of Pax5 at p35, EBi3 and Cd1d gene promoters. Naïve B cells isolated from orthotopic KPC mice were treated with αCD40/LPS and ±STAT3 inhibitor for 72 hr followed by ChIP analysis. FIG. 6 panel F) ChIP analysis for enrichment of H3K27ac at p35, EBi3 and Cd1d gene promoters. Naïve B cells were isolated and treated as in FIG. 6 panel E. FIG. 6 panel G) Experimental schematic used to investigate in vivo effect of STAT1 (STAT1i) and STAT3 (STAT3i) inhibition in B cells on pancreatic tumor growth. FIG. 6 panel H) Quantification of tumor weights from mice in FIG. 6 panel G. FIG. 6 panel I) Absolute number of intra-tumoral B cells from mice in FIG. 6 panel H. FIG. 6 panel J) Absolute number of intra-tumoral Breg cells from mice in FIG. 6 panel H. FIG. 6 panel K) Absolute number of intra-tumoral plasma cells from mice in FIG. 6 panel H. Error bars indicate SEM; p values were calculated using two-tailed; unpaired, Student t test. p values in FIG. 6 panels H to K were calculated using one-way ANOVA. NS: non-significant, *p<0.05, **p<0.005. Data are representative of three independent experiments.

FIG. 7 shows a schematic, bar graphs and data plots related to studies performed examining how BCL6 inhibition in tumor-educated naïve B cells restrains tumor growth. FIG. 7 panel A) Experimental schematic used to test in vivo effect of Bcl6 inhibition (Bcl6i) in B cells on pancreatic tumor growth. Naïve B cells from oKPC mice were treated with αCD40/LPS/IL35±Bcl6i and transferred into μMT mice, followed by orthotopic injection of KPC cells. FIG. 7 panel B) Quantification of tumor weights from μMT mice as in FIG. 7 panel A. FIG. 7 panels C-E) Absolute number of intra-tumoral B cells (FIG. 7 panel C) Breg cells (FIG. 7 panel D) and plasma cells (FIG. 7 panel E) in mice from FIG. 7 panel B. FIG. 7 panel F) Quantification of IgG and IgM levels in tumors from mice in FIG. 7 panel B. FIG. 7 panel G) Correlation between intra-tumoral Breg cells and IgG levels from mice in FIG. 7 panel B. FIG. 7 panel H) Fold change in expression of p35, EBi3 and IL-10 as determined by qPCR in intra-tumoral Breg cells isolated from mice in FIG. 7 panel B. FIG. 7 panel I) Frequency of intra-tumoral T effector (CD45+CD4+CD25−) and cytotoxic T cells (CD45+CD8+) in mice from FIG. 7 panel B. FIG. 7 panel J) Quantification of tumor weight from μMT mice 3 weeks post-orthotopic adoptive transfer of pre-treated B cells and intra-pancreatic injections with KPC cells as in FIG. 7 panel A±treatment with αPD-1 on days 7, 9 and 11. FIG. 7 panel K) Frequency of intra-tumoral cytotoxic T cells (CD45+CD8+) in mice from FIG. 7 panel J. FIG. 7 panel L) Quantification of tumor weight from μMT mice 3 weeks post-orthotopic adoptive transfer of pre-treated B cells and intra-pancreatic injections with KPC cells as in FIG. 7 panel J±depletion of CD8 T cells. Error bars indicate SEM; p values were calculated using two-tailed; unpaired, Student t test. NS: non-significant, *p<0.05, **p<0.005, ***p<0.001. Data are representative of three independent experiments. Experiments using CD8+ depletion were performed with 7-8-week-old μMT mice with at least 6 mice per group in duplicate.

FIG. 8 shows images of microscopy from studies performed relating to the detection of plasma and regulatory B cells in human PDAC, related to FIG. 1. Immunofluorescence staining for CD20+, CD138, pan-cytokeratin (CK) and Ebi3 in samples of human PDAC. Cytokeratin staining was used to distinguish cancer cells (CK+CD138+ from plasma cells CK-CD138 hi); similarly, CD138 and CD20 markers were used to distinguish Ebi3+ Breg from cancer and/or stromal cells. White box indicates the location of magnified images shown in FIG. 1 panel A. Scale bars, 100 μm.

FIG. 9 shows data plots from studies performed relating to the detection of plasma and regulatory B cells in human PDAC, related to FIG. 1. Data plots show results of studies performed on the correlation of cancer Breg signature with plasma cell signature in LUAD and COAD (TCGA) in relation to the analysis of plasma cell and Breg signatures in TCGA, also related to FIG. 1.

FIG. 10 shows data plots from studies performed examining the transcriptional regulation of naïve B cells in pancreatic cancer, related to FIG. 2. FIG. 10 panel A) Mean-difference (MD) plot showing differential expression between tumor bearing and non-tumor bearing WT naïve B cells. The y axis shows log 2 fold change, and the x axis shows average log 2 counts per million. Genes with significantly increased or decreased are shown (p-adj<0.05). FIG. 10 panel B) Mean-difference plot showing differential gene expression in naïve B cells from tumor bearing WT and BEBi3−/− mice. The y axis shows log 2 fold change, and the x axis shows average log 2 counts per million. Genes with significantly increased or decreased are shown (p-adj<0.05). FIG. 10 panel C) Volcano plot of genes that are differentially expressed (red; upregulated, blue; downregulated) with p-adj<0.05 in naive B cells from FIG. 10 panel B. FIG. 10 panel D) Representative flow cytometric plots for gating strategy of Breg (CD19+CD24hiCD38 hi) and Bcon cells (CD19+CD24loCD38lo) isolated from spleen of PDAC patients (left) and flow cytometric plots for intracellular expression of IL-35 from Bcon and Breg cells (right). FIG. 10 panels E and F) Quantification of IFN-γ and TNF-α from CD4+ T cells (FIG. 10 panel E) and CD8+ T cells (FIG. 10 panel F) co-cultured with activated Breg and Bcon cells isolated from spleens of PDAC patients. Data are representative of 3 independent experiments. Errors bars indicate SEM. NS: non-significant, **p<0.05, ***p<0.005 (Student t test, two-tailed, unpaired).

FIG. 11 shows blot plots and data graphs from studies performed indicating that IL-35 deficiency promotes intra-tumoral expansion of plasma cells, related to FIG. 3. FIG. 11 panel A) Validation of EBi3 (top) and p35 (bottom) loss in B cells from BEBi3−/− and Bp35−/− mice. Genomic DNA was isolated from CD19+ B cells, CD3+ T cells, non-labeled CD19−CD3− cells (Non-T, B). Genomic DNA samples from tails of WT and p35−/− mice were used as controls. B-actin gene was used as a standard. FIG. 10 panel B) Representative gating strategy for isolation of mouse regulatory (CD19+CD21hiCD5+CD1dhi) and naïve (CD19+IgDhiCD27−CD1d−) B cells. FIG. 11 panel C) Frequency of intra-tumoral CD45+ cells (gated on live cells) from tumors of WT, BEBi3−/− and Bp35−/− mice orthotopically injected with KPC cells and collected at 3 weeks after injection of tumor cells. FIG. 11 panel D) Frequency of splenic naïve B cells (% of CD45, left) and Breg cells (% of CD19, right) from tumor bearing WT, BEBi3−/− and Bp35−/− mice as determined by flow cytometry. FIG. 11 panel E) Correlation between intra-tumoral Breg cells and IgG levels from mice in FIG. 3 panel A. FIG. 11 panel F) Quantification of CD19loCD138 hi splenic plasma cells from non-tumor bearing mice performed by flow cytometry. FIG. 11 panel G) Quantification of tumor weights from WT, BEBi3−/− and Bp35−/− mice orthotopically injected with KPC 2173 cells and collected at 3 weeks after injection of tumor cells. Control WT mice represent both BEBi3+/− and BWT control bone marrow chimera groups. FIG. 11 panels H and I) Frequency of intra-tumoral naïve B cells (% of CD45; FIG. 11 panel H) and Breg cells (% of CD19; FIG. 11 panel I) as determined by flow cytometry from mice in FIG. 11 panel G. FIG. 11 panels J and K) Representative flow cytometry plots (FIG. 11 panel J) and quantification (FIG. 11 panel K) of CD19loCD138 hi intra-tumoral plasma cells from mice in FIG. 11 panel G. Numbers in flow plots indicate percent of plasma cells. FIG. 11 panels L and M) Representative flow cytometry plots (FIG. 11 panel L) and quantification (FIG. 11 panel M) of CD19loCD138 hi intra-tumoral plasma cells as determined by flow cytometry from spontaneous KPC (sKPC) mice treated with αPD-1, αIL-35 and αPD-1+αIL-35 with IgG control. Numbers in flow plots indicate percent of plasma cells. FIG. 11 panels N and O) Frequency of intra-tumoral naïve B cells (% of CD45; FIG. 11 panel N) and Breg cells (% of CD19; FIG. 11 panel O) as determined by flow cytometry from mice in FIG. 11 panel L. Error bars indicate SEM; p values were calculated using two-tailed; unpaired, Student t test. NS: non-significant, *p<0.01, **p<0.005, ***p<0.001. p values in FIG. 11 panels C, D, F and G to K were calculated using one-way ANOVA. NS: non-significant. p values in FIG. 11 panels M to O were calculated using two-way ANOVA. NS: non-significant, *p<0.01, **p<0.005, ***p<0.001. Experiments were performed using 7-8-week-old mice of indicated genotype and data are representative of three independent experiments with 12-14 mice per group.

FIG. 12 shows bar graphs from studies examining bortezomib treatment in mouse PDAC model, related to FIG. 3. FIG. 12 panel A) Percent cell viability of KPC 4662 cells treated for 48 hrs with indicated concentrations of bortezomib was determined by MTT assay. FIG. 12 panels B to D) Frequency of splenic total T cells (FIG. 12 panel B), B cells (FIG. 12 panel C), and CD11b+ cells (% of CD45; FIG. 12 panel D) from bortezomib and PBS treated WT or BEBi3−/− mice as determined by flow cytometry. FIG. 12 panel E) Frequency of intra-tumoral tumoral naïve B cells (% of CD45) from bortezomib and PBS treated WT or BEBi3−/− mice as determined by flow cytometry. FIG. 12 panel F) Frequency of intra-tumoral Breg cells (% of CD19) from bortezomib and PBS treated WT or BEBi3−/− mice as determined by flow cytometry. FIG. 12 panel G) Quantification of intra-tumoral effector CD4+CD25− T cells (% of CD45, left), CD8+ T cells (% of CD45, right) from bortezomib and PBS treated WT or BEBi3−/− mice 3 weeks post-orthotopic injection with KPC cells. Error bars indicate SEM; p values were calculated using two-tailed; unpaired, Student t test. NS: non-significant, *p<0.01, **p<0.005, ***p<0.001. p values were calculated using two-way ANOVA. NS: non-significant, *p<0.01, **p<0.005, ***p<0.001. Experiments were performed using 7-8-week-old mice of indicated genotype and data are representative of three independent experiments with 12-14 mice per group.

FIG. 13 shows bar graphs from studies analyzing tumor associated Breg cells having altered suppressive activity ex vivo, related to FIG. 4. FIG. 13 panel A) Expression of CD1d and IL-10 in naive B cells from WT (left; dark) and oKPC (right; gray) mice treated with LPS/αCD40±indicated cytokines for 72 hr as determined by flow cytometry. FIG. 13 panels B and C) Quantification of intracellular expression of pSTAT1 (FIG. 13 panel B) and pSTAT3 (FIG. 13 panel C) analyzed by flow cytometry in naive B cells derived from WT (left; dark) or oKPC (right; gray) mice and treated with LPS/αCD40±IL-35 at indicated time-points. FIG. 13 panel D) Quantification of intracellular expression of Pax5 analyzed by flow cytometry in LSP/αCD40 activated naïve B cells treated with IL-35 at different time-points (specified on x-axis) up to 72 hours for the indicated WT (left; dark) and oKPC (right; gray) genotype (MFI. mean fluorescence intensity). FIG. 13 panel E) Endogenous expression of Pax5 was quantified in Breg cells isolated from spleens and pancreatic tumors of non-tumor bearing and tumor bearing WT and BEBi3−/− mice. FIG. 13 panel F) Intracellular expression of pSTAT3 (left), Pax5 (middle), and BCL6 (left) analyzed by flow cytometry from intra-tumoral Breg cells isolated from WT, BEBi3−/− and Bp35−/− mice. FIG. 13 panel G) STAT3/Pax5 DNA binding motif within murine p35, Ebi3 and CD1d gene promoters was derived using Homer. FIG. 13 panels H and I) ChIP analysis for enrichment of pSTAT1 (FIG. 13 panel H) and pSTAT3 (FIG. 13 panel I) at p35, EBi3 and CD1d gene promoters. Naïve B cells isolated from WT and orthotopic KPC (oKPC) mice were treated with αCD40/LPS and rIL-35 for 72 hrs then used for downstream ChIP analysis. The relative enrichment of pSTAT1 and pSTAT3 to p35 promoter region −250, −1600, −1800 and −2200, EBi3 promoter region −250, −500, −1200 and −1600 and CD1d promoter region −80, −500 and −1300 upstream of TSS were shown and results are scaled to ChIP with control isotype antibody and input. FIG. 13 panels J and K) ChIP analysis for enrichment of pSTAT1 (FIG. 13 panel J) and pSTAT3 (FIG. 13 panel K) at p35, EBi3 and CD1d gene promoters. Naïve B cells isolated from WT and spontaneous KPC (sKPC) mice were treated with αCD40/LPS and rIL-35 for 72 hrs then used for downstream ChIP analysis. The relative enrichment of pSTAT1 and pSTAT3 to p35 promoter region −250, −1600, −1800 and −2200, EBi3 promoterregion −250, −500, −1200 and −1600 and CD1d promoter region −80, −500 and −1300 upstream of TSS were shown and results are scaled to ChIP with control isotype antibody and input. Error bars indicate SEM; p values were calculated using two-tailed; unpaired, Student t test. NS: non-significant, *p<0.05, **p<0.01, ***p<0.005. Experiments were performed using 7-8-week-old mice of indicated genotype and representative of three independent experiments.

FIG. 14 shows bar graphs from studies analyzing STATs and Pax5 binding to promoters of regulatory genes, related to FIG. 5. FIG. 14 panel A) ChIP analysis for enrichment of pSTAT1 and pSTAT3 at Pax5 promoter in naïve B cells (treated with αCD40/LPS/rIL-35 for 72 hr) from WT (left; dark) or spontaneous KPC (right; gray, sKPC) mice. Relative enrichment of pSTAT1 and pSTAT3 to Pax5 promoter region −1100 and −1600 upstream of TSS were shown and results are scaled to ChIP with control isotype antibody and input. FIG. 14 panel B) ChIP analysis for enrichment of pSTAT3 (left) and Pax5 (right) at BCL6 promoter. Naïve B cells were isolated and treated as in FIG. 14 panel A. FIG. 14 panel C) ChIP-re-ChIP analysis to detect STAT3-Pax5 complex at Bcl6 promoter in rIL-35 treated naïve B cells isolated from WT and spontaneous KPC (sKPC) mice. FIG. 14 panels D to F) ChIP analysis for enrichment of Pax5 at p35, EBi3 and CD1d gene promoter. Naïve B cells isolated from WT and orthotopic KPC (oKPC) mice were treated with αCD40/LPS and rIL-35 for 72 hr then used for downstream ChIP analysis. Relative enrichment of Pax5 on p35 promoter region −250, −1600, −1800 and −2200 (FIG. 14 panel D), EBi3 promoter region −250, −500, −1200 and −1600 (FIG. 14 panel E) and CD1d promoter region −80, −500 and −1300 (FIG. 14 panel F) upstream of TSS were shown and results are scaled to ChIP with control isotype antibody and input. FIG. 14 panels G to I) ChIP analysis for enrichment of Pax5 on p35, EBi3 and CD1d gene promoter. Naïve B cells isolated from WT and spontaneous KPC (sKPC) mice were treated with αCD40/LPS and rIL-35 for 72 hr then used for downstream ChIP analysis. Relative enrichment of Pax5 on p35 promoter region −250, −1600, −1800 and −2200 (FIG. 14 panel G), EBi3 promoter region −250, −500, −1200 and −1600 (FIG. 14 panel H) and CD1d promoter region −80, −500 and −1300 (FIG. 14 panel I) upstream of TSS were shown and results are scaled to ChIP with control isotype antibody and input. FIG. 14 panels J to K) ChIP-re-ChIP analysis to detect STAT3-Pax5 complex at promoters of p35 (FIG. 14 panel J), EBi3 (FIG. 14 panel K) and CD1d genes (FIG. 14 panel L) in rIL-35 treated naïve B cells isolated from WT or spontaneous KPC (sKPC) mice. FIG. 14 panels M to O) Mutation of pSTAT3 and Pax5 binding sites on the p35 (FIG. 14 panel M), EBi3 (FIG. 14 panel N) and CD1d (FIG. 14 panel O) gene promoter reduces the luciferase reporter activity in HEK 293T cells. Error bars indicate SEM; p values were calculated using two-tailed; unpaired, Student t test. NS: non-significant, **p<0.05, ***p<0.005. NS: non-significant, *p<0.05, **p<0.01 for ChIP experiments. The data represent triplicates within the same experiment and are representative of three independent experiments.

FIG. 15 shows bar graphs from studies analyzing B cell specific inhibition of STAT3 as modulating anti-tumor immunity, related to FIG. 6. FIG. 15 panel A) Intra-cellular levels of pSTAT1 and pSTAT3 in naïve B cells isolated from orthotopic KPC mice and treated with LPS/αCD40/rIL-35±STAT1 or STAT3 inhibitor for 72 hr. FIG. 15 panels B and C) Proliferation and percent cell viability of activated naïve B cells (FIG. 15 panel B) and Breg cells (FIG. 15 panel C) treated for 72 hrs with rIL-35 and STAT1 or STAT3 inhibitors as determined by MTT assay. FIG. 15 panels D to F) ChIP analysis for enrichment of Pax5 at p35 (FIG. 15 panel D), EBi3 (FIG. 15 panel E) and CD1d (FIG. 15 panel F) promoter. Naïve B cells isolated from KPC mice were treated with αCD40/LPS and rIL-35 in presence and absence of STAT1 inhibitor (STAT1i) for 72 hr, then used for downstream ChIP analysis. Relative enrichment of Pax5 were shown. FIG. 15 panels G to I) ChIP analysis for enrichment of H3K27ac at p35 (FIG. 15 panel G), EBi3 (FIG. 15 panel H) and CD1d (FIG. 15 panel I) gene promoters. Naïve B cells isolated from KPC mice were treated with αCD40/LPS and rIL-35 in presence and absence of STAT1 inhibitor for 72 hr then used for downstream ChIP analysis. Relative enrichment of H3K27ac is shown. FIG. 15 panel J) Frequency of intra-tumoral CD45+ cells (gated on live cells, top) and total B cells (gated on CD45+ cells, bottom) from μMT mice treated and injected as in FIG. 6 panel G, at 3 weeks post-orthotopic injection of KPC cells. FIG. 15 panels K and L) Representative flow cytometry plots (FIG. 15 panel K) and quantification (FIG. 15 panel L) of intra-cellular expression of IL-10, p35 and EBi3 in intra-tumoral Breg cells isolated from μMT mice treated and injected as in FIG. 6 panel H, at 3 weeks post-orthotopic injection of KPC cells. FIG. 15 panel M) Frequency of intra-tumoral T effector (CD45+CD4+CD25−), cytotoxic (CD45+CD8+) T cells in μMT mice from FIG. 6 panel H. FIG. 15 panels N and O) Representative flow cytometry plots (FIG. 15 panel N) showing frequency of intra-tumoral regulatory T cells and IFN-7+CD8+ T cells; FIG. 15 panel O provides quantification thereof in mice from FIG. 6 panel H. FIG. 15 panel P) T effector to Treg and T cytotoxic to Treg ratio in μMT mice from FIG. 6 panel H. Error bars indicate SEM; p values were calculated using two-tailed; unpaired, Student t test. NS: non-significant, *p<0.01, **p<0.005, ***p<0.001. p values in FIG. 15 panels J to P were calculated using one-way ANOVA. NS: non-significant, *p<0.01, **p<0.001, ***p<0.0001. Data are representative of three independent experiments.

FIG. 16 shows bar graphs of studies examining BCL6 inhibition in B cells as restraining PDAC growth, related to FIG. 7. FIG. 16 panels A and B) Proliferation and percent cell viability of activated naïve B cells (FIG. 16 panel A) and Breg cells (FIG. 16 panel B) treated for 72 hr with rIL-35 and BCL6 inhibitor as determined by MTT assay. FIG. 16 panel C) Quantification of p35, EBi3 and CD1d expression by naïve B cells treated for 72 hr with rIL-35 and BCL6 inhibitor as determined by qPCR. FIG. 16 panel D) Immunohistochemical detection of CD138+ plasma cells in tumor tissues from μMT mice adoptively transferred with B cells as in FIG. 7 panel A. Scale bars, 1000 μm. FIG. 16 panel E) Quantification of intra-tumoral CD138+ plasma cells per field of view (FOV) from mice in FIG. 16 panel D, as determined by immunohistochemistry. FIG. 16 panel F) Representative flow cytometric plots for CD8+ T cells from αPD-1, αCD8 and αPD-1+αCD8 treated MT mice 3 weeks post-orthotopic adoptive transfer of pre-treated B cells as in FIG. 7 panel J and intra-pancreatic injections with KPC cells. FIG. 16 panels G and H) Absolute number of plasma cells (FIG. 16 panel G) and Breg cells (FIG. 16 panel H) from MT mice treated as in FIG. 16 panel F. FIG. 16 panel I) Frequency of intra-tumoral CD8+ T cells (% of CD45) in PMT mice treated as in FIG. 16 panel F. Error bars indicate SEM; p values were calculated using two-tailed; unpaired, Student t test. NS: non-significant, *p<0.05, **p<0.005. Data are representative of three independent experiments. Experiments were performed using 6-8-week-old mice of indicated genotype with at least 9-12 mice per group in triplicate. Experiments using in vivo BCL6 inhibition in B cells with CD8 depletion were performed using 7-8-week-old μMT mice with at least 6 mice per group in duplicate.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

“Amino acid sequence” and terms such as “peptide,” “polypeptide,” and “protein” are used interchangeably herein, and are not meant to limit the amino acid sequence to the complete, native amino acid sequence (i.e., a sequence containing only those amino acids found in the protein as it occurs in nature) associated with the recited protein molecule. The proteins and protein fragments of the presently disclosed subject matter can be produced by recombinant approaches or can be isolated from a naturally occurring source. The protein fragments can be any size, and for example can range in size from four amino acid residues to the entire amino acid sequence minus one amino acid.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

A “nucleic acid,” “nucleic acid molecule,” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

A “therapeutic,” “therapeutic polypeptide,” “therapeutic molecule” and similar terms refer to a polypeptide and/or molecule that can alleviate, reduce, prevent, delay and/or stabilize symptoms that result from an absence or defect in a protein in a cell or subject and/or is a polypeptide and/or molecule that otherwise confers a benefit to a subject, e.g., anti-cancer effects or improvement in transplant survivability or induction of an immune response.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of cancer.

As used herein, the terms “reduce,” “reduces,” “reduction,” “diminish,” “inhibit” and similar terms mean a decrease of at least about 5%, 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

By “substantially retain” a property and/or to maintain a property “substantially the same” as a comparison (e.g., a control), it is meant that at least about 75%, 85%, 90%, 95%, 96%, 97%, 98%, 99% or 100% of the property (e.g., activity or other measurable characteristic) is retained.

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. Preferably, the subject is a human. The subject may be of any gender, any ethnicity and any age. In some embodiments, the subject has, is suspected to have, and/or is at risk of cancer, e.g., a “subject in need”.

A “subject in need thereof” or “a subject in need of” is a subject known to have, or is suspected of having or developing or is at risk of having or developing disorder that can be treated by the methods and compositions of the present invention, or would benefit from the delivery of a particle and/or composition including those described herein.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compositions of the invention for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset are substantially less than what would occur in the absence of the present invention.

A “treatment effective” or “effective” amount as used herein is an amount that is sufficient to provide some improvement or benefit to the subject. Alternatively stated, a “treatment effective” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

An “effective amount” or “therapeutically effective amount” refers to an amount of a compound or composition of this invention that is sufficient to produce a desired effect, which can be a therapeutic and/or beneficial effect. In general, a “therapeutically effective amount” or “treatment effective amount” refers to an amount that is a sufficient, but non-toxic, amount of the active ingredient (i.e., particles of this invention) to achieve the desired effect, which, for example, can be a reduction or elimination in the severity and/or frequency of symptoms and/or improvement or remediation of damage, or otherwise prevent, hinder, retard or reverse the progression of a disease or any other undesirable symptom. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in B cells with anti-tumor functions (e.g., enhanced antibody production, e.g., production of immunosuppressive cytokines, e.g., enhanced T cell interaction and/or activation, e.g., plasma B cell differentiation, e.g., reduced B regulatory cell (“Breg”) differentiation, e.g., reduced IL10, IL35, and/or TGFβ expression). Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to antigens the host views as “non-self”, e.g., immunogens by infection or by vaccination, e.g., cancer antigens. Active immunity can be contrasted with passive immunity, which is acquired through the “transfer of preformed substances (antibody, transfer factor, thymic graft, interleukin-2) from an actively immunized host to a non-immune host.” Id.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

As used herein, “immunosuppressive” refers to a function or activity that suppresses one or more aspects of an active immune response. An “immunosuppressive agent” refers to an agent that inhibits or prevents an immune response, e.g., to a foreign material in a subject. Immunosuppressive agents generally act by inhibiting immune cell (e.g., T-cell, B-cell, NK cell, and the like) activation, disrupting proliferation, and/or suppressing inflammation.

The term “biologically active” as used herein means an enzyme or protein having structural, regulatory, or biochemical functions of a naturally occurring molecule.

The terms “antibody” and “immunoglobulin” include antibodies or immunoglobulins of any isotype, fragments of antibodies that retain specific binding to antigen, including but not limited to Fab, Fv, single chain Fv (scFv), Fc, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen (e.g., any antibody fragment that comprises at least one paratope).

Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include antibodies, antibody fragments, and nucleic acid sequences.

As used herein, the term “sample” is used in its broadest sense. In one sense, it is meant to include a specimen from a biological source. A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art. For example, biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject.

As used herein the term “control” refers to a comparative sample and/or other reference source for a control subject.

“Control subject” as used herein refers to a subject which does not have said condition(s) of the subject in need, e.g., said cancer and/or an illness to which the methods of the present invention disclosed herein may provide beneficial health effects.

As used herein, “tumor microenvironment” or “TME” refers to the immediate and adjacent surroundings of a tumor, including but not limited to, the surrounding stromal environment (e.g., stroma cells), vasculature (e.g., blood vessels), immune cells, fibroblasts and extracellular matrix (ECM), as well as any molecules (e.g., proteins or other macromolecules) encompassed therein. Further description of tumor microenvironments can be found in, e.g., Valkenburg et al. 2018 Nat Rev Clin Oncol 15:366-381 and Ho et al. 2020 Nat Rev Clin Oncol 17:527-540, the disclosures of each of which are incorporated herein by reference.

The term “reprogrammed” and/or “reprogram” as used herein refers to a set of epigenetic, transcriptional and/or translational modifications induced in the cell leading to a lineage change (“reprogramming” from one lineage to another). A B cell cultured under growth conditions inducing such epigenetic, transcriptional and/or translational modifications leading to a lineage change may be referred to as a “reprogrammed B cell.” For example, a B cell (e.g., an immunosuppressed B cell, e.g., a naïve B cell, e.g., a “tumor-educated” B cell) may be cultured in conditions comprising IL35 cytokine and/or Bcl-6 inhibitor and become reprogrammed into an anticancer B cell (e.g., an anticancer plasma B cell).

The term “reprogramming factors” as used herein refers to any factors (e.g., exogenous or endogenous factors) required in the culture conditions as needed to induce the lineage change (i.e., the reprogramming). Non-limiting examples of reprogramming factors include IL35, STAT3 inhibitor, and/or Bcl-6 inhibitor.

Without wishing to be bound to theory, many types of solid malignancies, including pancreatic cancer, select for robust interactions between tumor cells and host responses that establish markedly immunosuppressive environment with influx of myeloid cells, Treg cells, activation of cancer-associated fibroblasts and expansion of regulatory T and B cell responses (Michaud et al. 2020 Immunological Reviews 299:74-92).

The balance between regulatory and plasma B cells in the tumor immune microenvironment can determine sensitivity to immune checkpoint inhibitors, yet the mechanisms that govern B cell differentiation in the context of tumorigenesis are poorly characterized. Tumor-promoting B cells are typically defined by their ability to modulate immune tolerance via production of immunosuppressive cytokines and/or direct interaction with T cells (Michaud et al. 2020 Immunological Reviews 299:74-92; Shen and Fillatreau 2015 Nature Reviews Immunology 15:441-451). On the other hand, in both oncologic and autoimmune diseases, immunoregulatory B cells (Bregs) can be found as diverse IL10+, IL35+ and/or TGFβ+ B cell populations (He et al. 2014 J. Immunology Research 2014; Shen et al. 2014 Nature 507:366-370; Wang et al. 2014 Nature Medicine 20:633-641; Pylayeva-Gupta et al. 2016 Cancer Discovery 6:247-255; Yanaba et al. 2008 Immunity 28:639-650; Korniotis et al. 2016 Nature Communications 7:1-13; Cherukuri et al. 2021 Immunological reviews 299:31-44; Mirlekar et al. 2018 Cancer Immunology Research 6:1014-1024; Mirlekar et al. 2020 Cancer Immunology Research 8:292-308.

The transcriptional and signaling mechanisms that regulate B cell differentiation in malignancy are also not well understood, and there is a significant knowledge gap in the understanding of how effective anti-tumor B cell responses versus regulatory responses are generated. As such, there is a need to develop new therapeutic methods for the management of overcoming immunosuppression in cancers, and strategies to enhance anti-tumor B cell responses are needed (Largeot et al. 2019 Cells 8:449).

Accordingly, one aspect of the present invention comprises a method of producing a reprogrammed B cell, comprising culturing a B cell in a medium with a reprogramming factor to produce a reprogrammed B cell.

Another aspect of the present invention comprises a method of producing a population of reprogrammed B cells, comprising culturing a B cell in a medium with a reprogramming factor to produce a population of one or more reprogrammed B cells.

In some embodiments, the culturing may be in vitro culturing, e.g., culturing of in vitro derived B cell(s), such as but not limited to culturing an immortalized B cell line.

In some embodiments, the culture may be ex vivo culturing, e.g., culturing of B cell(s) derived from a sample and/or a subject, e.g., an ex vivo B cell. In some embodiments, the B cell may comprise an ex vivo isolated B cell derived from a subject (e.g., a human patient).

Another aspect of the present invention comprises a method of treating a cancer in a subject, comprising delivering to the subject an effective amount of the reprogrammed B cell and/or B cell population of the invention.

Another aspect of the present invention comprises a method of enhancing an immune response to cancer in a subject, comprising delivering to the subject an effective amount of the reprogrammed B cell and/or B cell population of the invention.

Any immune response and/or aspect of an immune response may be enhanced from the methods of the present invention. In some embodiments, a B and/or a T cell response may be enhanced. In some embodiments, anti-cancer immune responses, e.g., anti-cancer B and/or T cell responses may be enhanced.

Accordingly, another aspect of the present invention provides a method of treating a cancer in a subject, comprising the steps of: (a) culturing a B cell (e.g., ex vivo culturing) in a medium with a reprogramming factor to produce a population of one or more reprogrammed B cells; and (b) delivering the reprogrammed B cells to the subject, thereby treating the cancer in the subject.

Another aspect of the present invention provides a method of enhancing an immune response (e.g., a B and/or T cell response) to cancer in a subject, comprising the steps of: (a) culturing a B cell (e.g., ex vivo culturing) in a medium with a reprogramming factor to produce a population of one or more reprogrammed B cells; and (b) delivering the reprogrammed B cells to the subject, thereby the immune response to the cancer in the subject.

The reprogrammed B cell may be reprogrammed to have any one or more anti-tumor functions. In some embodiments, reprogrammed B cells may comprise anti-tumor functions including, but not limited to, enhanced antibody production, enhanced production of immunosuppressive cytokines, enhanced T cell interaction and/or activation, enhanced plasma B cell differentiation, enhanced reduced B regulatory cell (“Breg”) differentiation, and/or reduced IL10, IL35, and/or TGFβ cytokine expression.

In some embodiments, the reprogrammed B cells are and/or differentiate into plasma B cells.

In some embodiments, the reprogramming factor may comprise one or more STAT3 pathway activators and/or inhibitors. Non-limiting examples of reprogramming factors of the present invention include LPS, anti-CD40, a STAT3 inhibitor (e.g., STA-21), a Bcl-6 inhibitor (e.g., Bcl6i 79-6), and any combination thereof.

The culturing step of the methods of the invention may be performed using any standard technique commonly used in the field. In some embodiments, the culturing may comprise incubating the B cell with the reprogramming factor for about 30 minutes to about 96 hours, e.g., about 30, 35, 40, 45, 50, 55, 60, 65, 70, 75, 80, 85, 90, 95, 100, 105, 110, 115, or 120 minutes (2 hrs), or about 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, or 96 hrs (4 days), or about 1, 2, 3, 4, 5, or 6 days or more, or any value or range therein. For example, in some embodiments, the culturing may comprise incubating the B cell with the reprogramming factor for about 30 minutes to about 96 hrs, about 1 hr to about 48 hrs, about 45 minutes to about 90 minutes, about 12 hrs to about 72 hrs, or about 30 minutes, about 1 hr, about 90 minutes, about 12 hrs, about 48 hrs, about 72 hrs, about 1 day, or about 5 days.

In some embodiments, the culturing may comprise incubating the B cell with a reprogramming factor in a concentration of about 1 μmol/L to about 1000 μmol/L or any value or range therein, e.g., in a concentration of about 1, 2, 3, 4, 5, 6, 7, 8, 9, 10, 15, 20, 30, 40, 5, 60, 70, 80, 90, 100, 125, 150, 175, 200, 250, 300, 350, 400, 450, 500, 550, 600, 650, 700, 750, 800, 850, 900, 950, 975, 990, 995, or about 1000 μmol/L. For example, in some embodiments, the culturing may comprise incubating the B cell with the reprogramming factor in a concentration of about 1 μmol/L to about 500 μmol/L, about 10 μmol/L to about 1000 μmol/L, about 50 μmol/L to about 995 μmol/L, or about 1 μmol/L, about 10 μmol/L, about 75 μmol/L, about 325 μmol/L, about 650 μmol/L, about 750 μmol/L, or about 1000 μmol/L, or any value or range therein.

In some embodiments, the culturing may comprise incubating the B cell with a reprogramming factor in a concentration of about 1 ng/ml to about 10 μg/ml, or any value or range therein, e.g., in a concentration of about 1, 2, 3, 4, 5, 6, 7, 8, 9, 10, 15, 20, 30, 40, 5, 60, 70, 80, 90, 100, 125, 150, 175, 200, 250, 300, 350, 400, 450, 500, 550, 600, 650, 700, 750, 800, 850, 900, 950, 975, 990, 995, or about 1000 ng/ml, or about 0.25, 0.5, 0.75, 1, 1.25, 1.5, 1.75, 2, 2.5, 3, 3.5, 4, 4.5, 5, 5.5, 6, 6.5, 7, 7.5, 8, 8.5, 9, 9.25, 9.5, 9.75, 9.9, or 10 μg/ml. For example, in some embodiments, the culturing may comprise incubating the B cell with the reprogramming factor in a concentration of about 1 ng/ml to about 500 ng/ml, about 10 ng/ml to about 1000 ng/ml, about 50 ng/ml to about 995 ng/ml, or about 1 ng/ml, about 10 ng/ml, about 75 ng/ml, about 325 ng/ml, about 650 ng/ml, about 750 ng/ml, or about 1000 ng/ml, or any value or range therein. In some embodiments, the culturing may comprise incubating the B cell with the reprogramming factor in a concentration of about 1 ng/ml to about 5 μg/ml, about 10 ng/ml to about 10 μg/ml, about 50 ng/ml to about 9.5 μg/ml, or about 1 μg/ml, about 10 μg/ml, about 7.5 μg/ml, about 3.25 μg/ml, about 6.5 μg/ml, about 7 μg/ml, or about 9.5 μg/ml, or any value or range therein.

The B cell of the present invention may be source and/or derived from any type of B cell type and any type of source, e.g., in vitro, ex vivo, and/or in vivo. For example, in some embodiments, the B cell may be a liver-sourced B cell, including but not limited to, e.g., a liver-sourced immortalized B cell line, e.g., an ex vivo B cell isolated from the liver of a subject and/or a liver sample (e.g., a biopsy). In some embodiments, the B cell may be a blood-circulatory-sourced B cell, including but not limited to, e.g., a blood-sourced immortalized B cell line, e.g., an ex vivo B cell isolated from blood of a subject and/or a blood sample.

In some embodiments, B cell may be sourced (e.g., isolated) from a cancer proximal environment (e.g., cancer microenvironment).

In some embodiments, the B cell may be sourced (e.g., isolated) from a cancer nonproximal environment (e.g., sourced from an environment distal from a cancer, e.g., not sourced from a cancer microenvironment).

In some embodiments, the subject may have, be suspected to have, and/or be at risk of cancer. A cancer relevant to the present invention may be any cancer in which the tumor microenvironment may comprise B cells. In some embodiments, the cancer may be any cancer such as, but not limited to, pancreatic cancer (e.g., pancreatic adenocarcinoma (PAAD), pancreatic ductal adenocarcinoma (PDAC), and the like), skin cancer (e.g., melanoma, e.g., skin cutaneous melanoma (SKCM) and the like), lung cancer (e.g., Lung adenocarcinoma (LUAD), lung squamous cell carcinoma (LUSC), and the like), mesothelioma (MESO; e.g., pleural mesothelioma, peritoneal mesothelioma, pericardial mesothelioma, testicular mesothelioma, and the like), breast cancer (e.g., breast invasive carcinoma (BRCA) and the like), blood cancer (e.g., lymphoid neoplasm diffuse large B-cell lymphoma (DLBC), acute myeloid leukemia (LAML), chronic myelogenous leukemia (LCML), and the like), adrenocortical carcinoma (ACC), bladder urothelial carcinoma (BLCA), brain cancer (e.g., brain lower grade glioma (LGG), glioblastoma multiforme (GBM), Head and Neck squamous cell carcinoma (HNSC), and the like) cervical squamous cell carcinoma and endocervical adenocarcinoma (CESC), cholangiocarcinoma (CHOL), colon adenocarcinoma (COAD), esophageal carcinoma (ESCA), kidney or liver cancer (e.g., kidney chromophobe (KICH), kidney renal clear cell carcinoma (KIRC), kidney renal papillary cell carcinoma (KIRP), liver hepatocellular carcinoma (LIHC), and the like), ovarian serous cystadenocarcinoma, pheochromocytoma and paraganglioma (PCPG), prostate adenocarcinoma (PRAD), rectum adenocarcinoma (READ), sarcoma (SARC), stomach adenocarcinoma (STAD), testicular germ cell tumors (TGCT), thymoma (THYM), thyroid carcinoma (THCA), uterine carcinosarcoma (UCS), uterine corpus endometrial carcinoma (UCEC), and/or uveal melanoma (UVM). In some embodiments, the cancer may be a solid cancer (e.g., a tumor).

In some embodiments, the cancer may be pancreatic ductal adenocarcinoma (PDAC)).

In some embodiments, the cancer may be a liquid cancer (e.g., leukemia and the like).

In some embodiments, the methods of the present invention may further comprise a step of isolating the B cell from the subject prior to ex vivo culturing.

In some embodiments, delivering the reprogrammed B cell(s) (e.g., delivering an effective amount of the reprogrammed B cell(s)) may comprise at least one or more iterative administrations of the reprogrammed B cell, e.g., administering the reprogrammed B cell every 1, 2, 3, 4, 5, 6, 7, 8, 9, 10, 11, 12, or more weeks, e.g., administering the reprogrammed B cell every 1, 2, 3, 4, 5, 6, 7, 8, 9, 11, 12 or more months, or any value or range therein. For example, in some embodiments, delivering the reprogrammed B cell(s) may comprise serial administrations of the reprogrammed B cell(s) every 1, 2, 3, 4, 5, 6, 7, 8, 9, 10 or more days (e.g., every 3 days, every 7 days, once a week, twice a week, three times a week etc.), for one or more repeats (e.g., every 3 days repeated for 4 times, twice a week repeated for 3 weeks etc.). In some embodiments, the reprogrammed B cell(s) may be administered on one schedule (e.g., every 3 days repeated for 4 times, etc.).

In some embodiments, the methods of the present invention may further comprise co-delivering a therapeutic agent to the subject.

In some embodiments, co-delivering of a therapeutic agent to the subject may comprise concurrent delivery of the therapeutic agent and the reprogrammed B cell(s).

In some embodiments, concurrent delivery (e.g., co-delivering) of the therapeutic agent and the reprogrammed B cell(s) may comprise delivering the therapeutic agent prior to (e.g., about 1 to about 24 hours prior to, about 1 to about 7 days prior to, about 1 to about 4 weeks prior to) delivery of the reprogrammed B cells.

In some embodiments, concurrent delivery (e.g., co-delivering) of the therapeutic agent and the reprogrammed B cell(s) may comprise delivering the therapeutic agent after (e.g., about 1 to about 24 hours after, about 1 to about 7 days after, about 1 to about 4 weeks after) delivery of the reprogrammed B cells.

The therapeutic agent may be any cancer treatment which may be of use to the subject in need. In some embodiments, the therapeutic agent may be an immunotherapy agent (e.g., checkpoint inhibitor, e.g., anti-PD1, anti-PDL1, anti-CTLA4, and the like). In some embodiments, the immunotherapy may be an autologous cellular immunotherapy, e.g., chimeric antigen receptor (CAR)-T cell therapy, CAR-NK cell therapy, and/or other modified immune cell (e.g., dendritic cell based therapy, e.g., Sipuleucel-T and the like)). In some embodiments, the immunotherapy may be targeted antibody therapy (e.g., monoclonal antibody therapy) such as, but not limited to, anti-CD20, anti-EGFR, anti-VEGF, anti-VEGFR2, anti-TNFα, anti-CD44, anti-CD19, anti-CD3, anti-EpCAM, anti-IGF1R, anti-MUC1, anti-CD51, anti-integrin, or any other targeted antibody-based therapy with anti-cancer function.

In some embodiments, the checkpoint inhibitor therapy may be inhibitors targeting CTLA-4, PD-1 and/or PD-L1. In some embodiments, the checkpoint inhibitor therapy may be, but is not limited to, pembrolizumab, nivolumab, ipilimumab, atezolizumab, avelumab, durvalumab, cemiplimab, amivantamab, apolizumab, bevacizumab, bivatuzumab, blinatumomab, camrelizumab, catumaxomab, cemiplimab, cixutumumab, clivatuzumab (e.g., clivatuzumab tetraxetan) durvalumab, edrecolomab, ertumaxomab, etaracizumab, faricimab, inebilizumab, intetumumab, isatuximab, margetuximab, necitumumab, nimotuzumab, Obinutuzumab, ocrelizumab, ofatumumab, olaratumab, panitumumab, pemtumomab, pertuzumab, racotumomab, ramucirumab, retifanlimab, rituximab, siltuximab, tafasitamab, teclistamab, tisotumab, tositumomab, trastuzumab, tremelimumab, votumumab and/or any variant or biosimilar thereof.

In some embodiments, the therapeutic agent may be surgery. In some embodiments, the therapeutic agent may be radiation therapy.

The invention also provides compositions (e.g., immunogenic compositions) use in the methods of the invention, e.g., compositions comprising a reprogrammed B cell produced by any one of the methods of the invention and/or for use in any one of the methods of the invention. The composition can further comprise a pharmaceutically acceptable carrier, diluent, and/or adjuvant.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

According, one aspect of the present invention provides a composition comprising one or more reprogrammed B cell(s) produced by any one of the methods of the invention.

Another aspect of the present invention provides a composition comprising one or more reprogrammed B cell(s) for use in any one of the methods of the invention.

In some embodiments, the present invention provides a pharmaceutical composition comprising one or more reprogrammed B cell(s) of the invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Pharmaceutical compositions comprising one or more reprogrammed B cell(s) of the invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition compositions comprising one or more reprogrammed B cell(s) of the invention, and a pharmaceutically acceptable carrier may further comprise an adjuvant. As used herein, “suitable adjuvant” describes an adjuvant capable of being combined with compositions comprising one or more reprogrammed B cell(s) of the invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

Also provided herein is a kit comprising a composition of the present invention, and optional instructions for the use thereof.

Kits that include particles of this invention and/or a pharmaceutical composition as described herein are also provided herein. Some kits include particles and/or compositions in a container (e.g., vial or ampule), and may also include instructions for use of the particles and/or composition in the various methods disclosed above. The particles and/or composition can be in various forms, including, for instance, as part of a solution or as a solid (e.g., lyophilized powder). The instructions may include a description of how to prepare (e.g., dissolve or resuspend) the particles in an appropriate fluid and/or how to administer the particles for the treatment of the diseases and disorders described herein.

The kits may also include various other components, such as buffers, salts, complexing metal ions and other agents described above in the section on pharmaceutical compositions. These components may be included with the chimeric protein or may be in separate containers. The kits may also include other therapeutic agents for administration with the chimeric protein. Examples of such agents include, but are not limited to, agents to treat the disorders or conditions described above.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

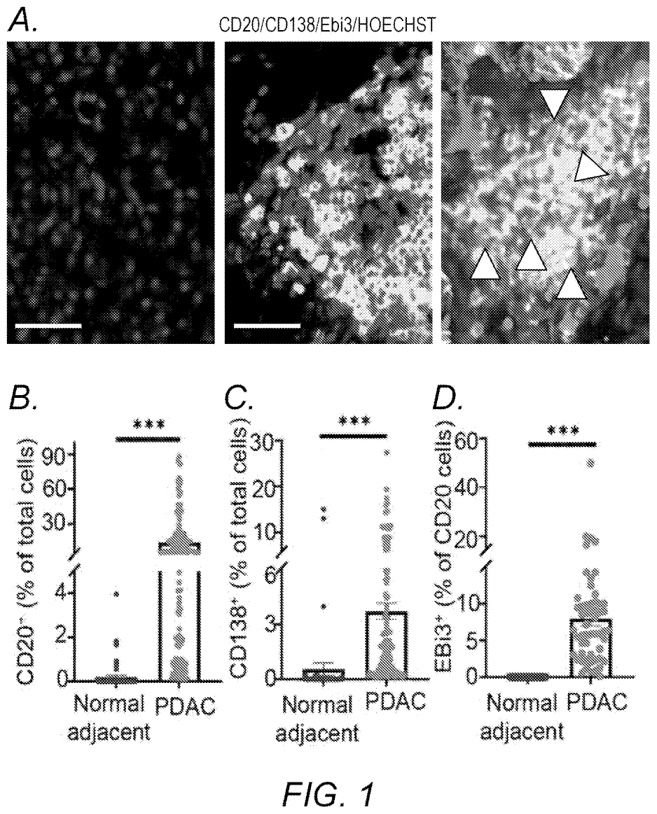

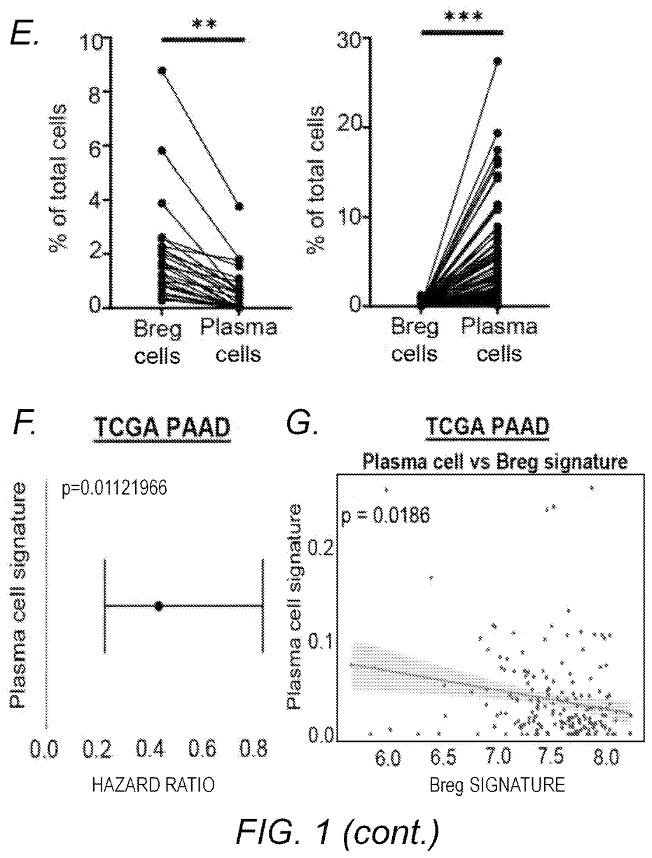

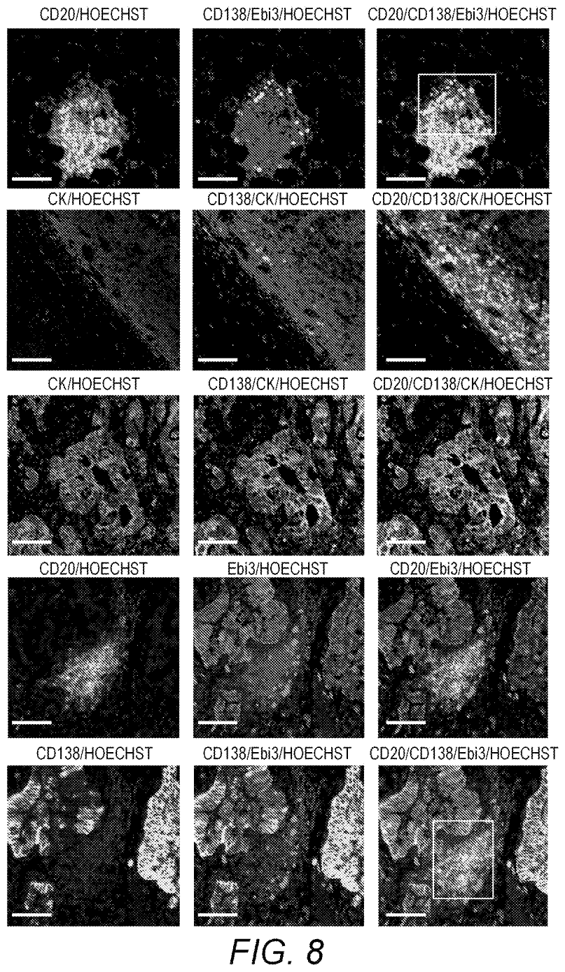

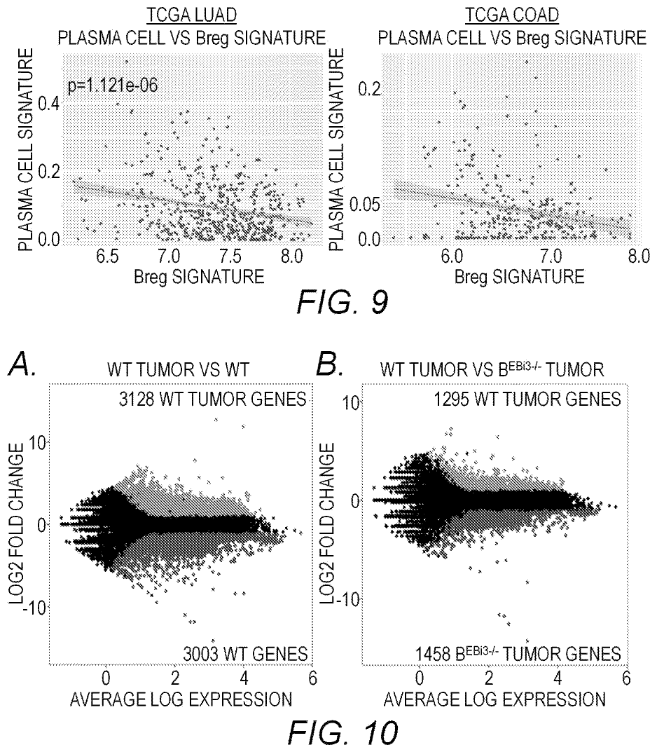

The distribution of plasma and immunoregulatory B cells was assessed in order to characterize the relationship between B cell subtypes in PDAC patients. To quantify abundance, multiplex immunofluorescence was used to analyze primary human PDAC tissue samples for the presence of CD20⁺ B cells, CK-CD138⁺ plasma cells and CD20⁺Ebi3⁺ regulatory B cells (Breg) (FIG. 1 panel A and FIG. 8). Compared to normal adjacent tissues, PDAC samples had increased numbers of total B cells, as well as increases in overall plasma and Breg abundance (FIG. 1 panels B-D). Analysis of relative frequencies of plasma and Breg cells in each sample revealed a significant negative correlation (FIG. 1 panel E). Frequency of plasma cells may be linked to improved survival prognosis as seen in melanoma and ovarian cancers (Kroeger et al. 2016 Clinical Cancer Research 22: 3005-3015; Gupta et al. 2019 Cancers 11: 894; Bosisio et al. 2016 Modern Pathology 29:347-358; Verma and Kumar 2020 Frontiers in Immunology 11:979). In this study, to better understand how plasma cell frequency related to survival in patients with pancreatic cancer, reported plasma and regulatory B cell signatures in patients with PDAC (PAAD) from The Cancer Genome Atlas (TCGA) were evaluated. It was found that plasma cell signature correlated with better survival (FIG. 1 panel F). Consistent with immunofluorescence-based quantification, a significant negative correlation was observed between regulatory and plasma cell signatures in PAAD (FIG. 1 panel G) as well as patients in lung and colorectal adenocarcinoma cohorts (LUAD and COAD respectively, FIG. 9). Collectively, these data demonstrate a positive correlation between plasma cell abundance and prognosis in PDAC and reveal a negative relationship between regulatory and plasma cell abundance in several major cancer types. These observations raised the possibility that B cell differentiation programs may be altered in cancer.

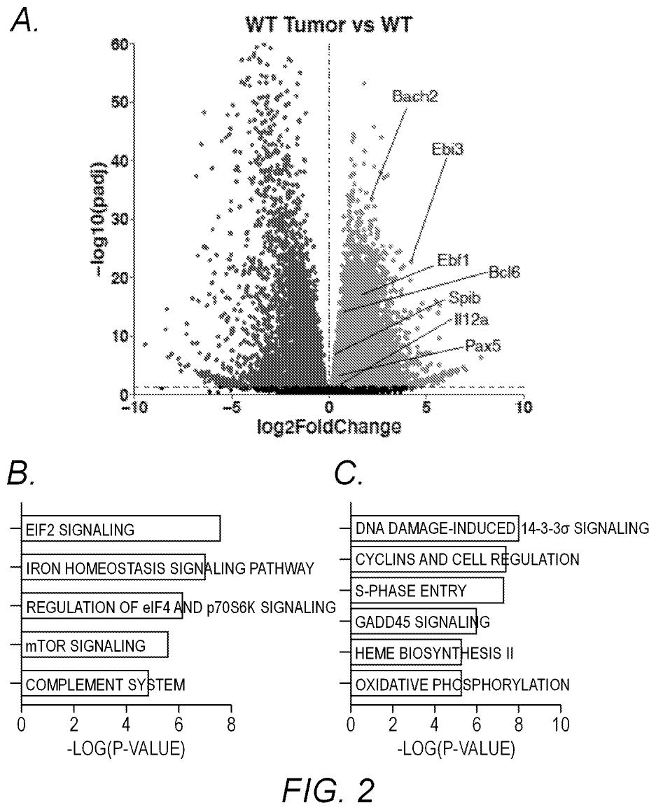

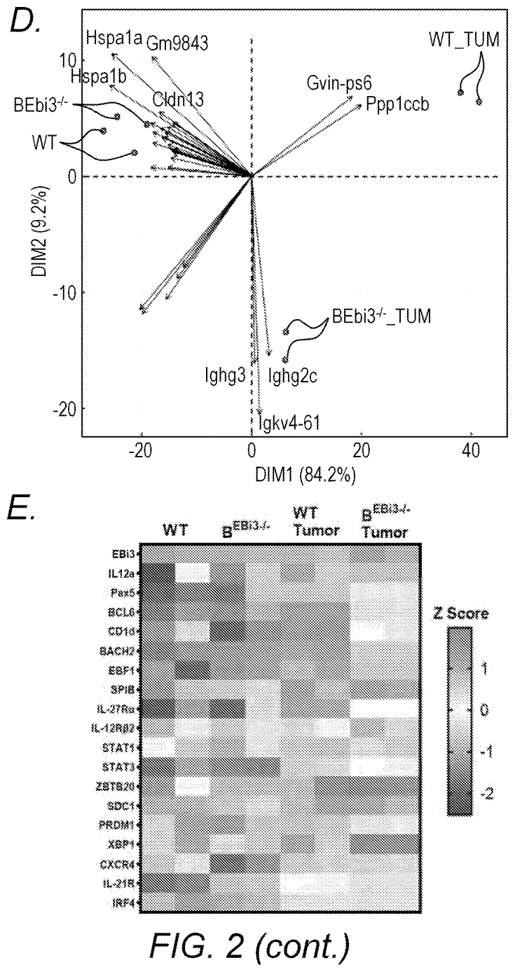

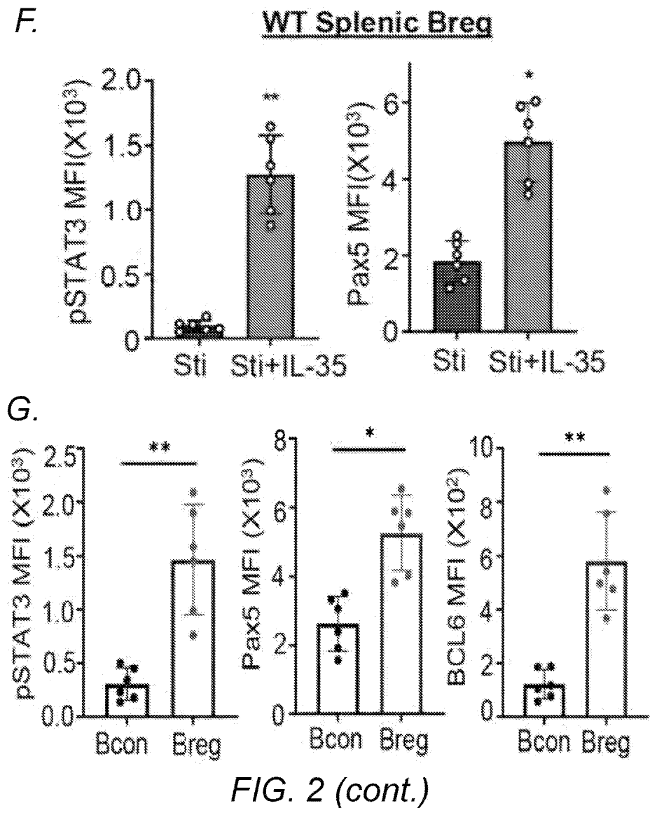

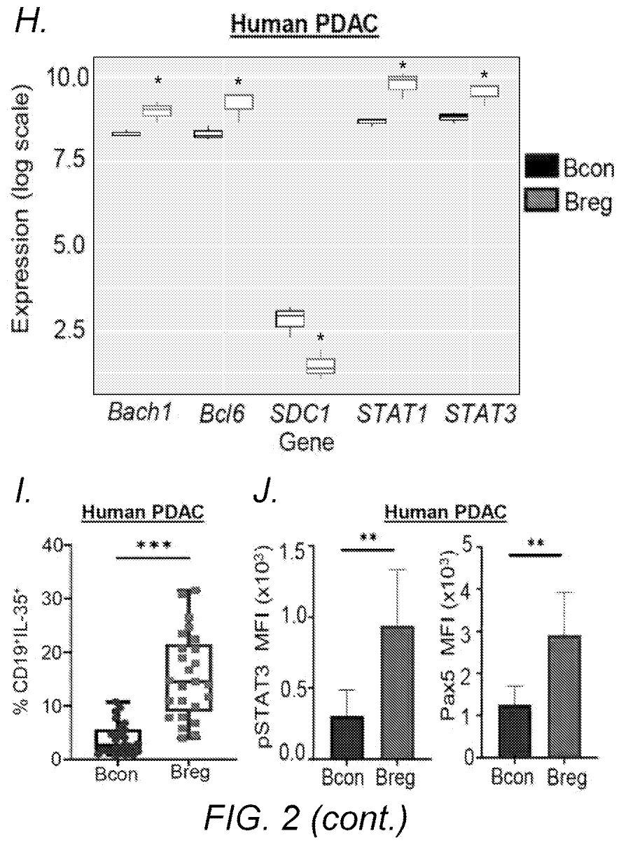

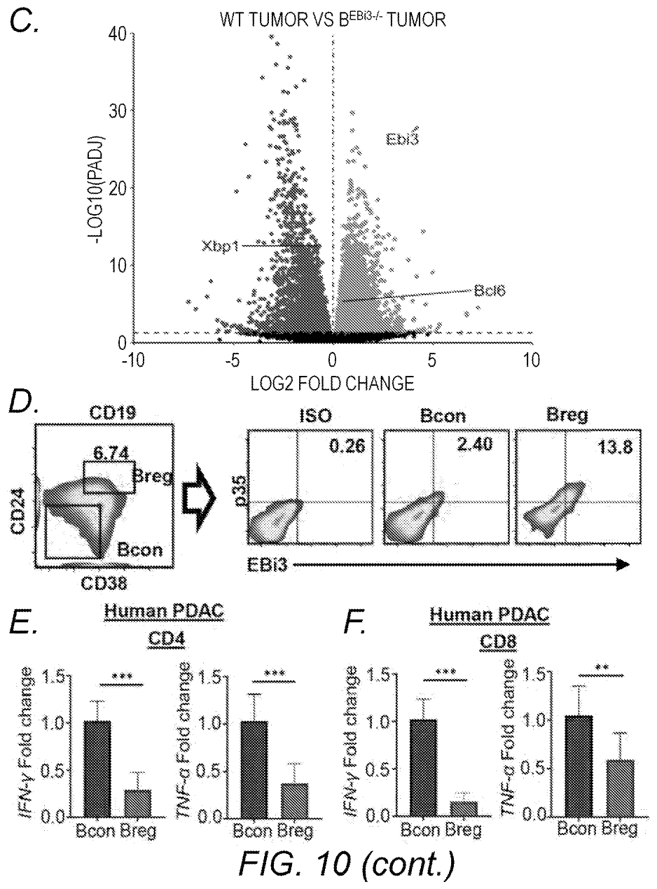

Changes in transcriptional profile of tumor-educated naïve B cells: To characterize molecular changes to naïve B cell in cancer, RNAseq was conducted of primary naïve B cells from control or orthotopic tumor-bearing mice (tumor cells contain mutations in Kras^{G12D/+}; TP53^{R172H/+}; p48^{Cre/+} (oKPC)). Altogether, 6131 genes were differentially regulated between wild-type (WT) and tumor-educated naïve B cells (FIG. 2 panel A, FIG. 10 panel A). Functional annotation of genes in tumor-educated naïve B cells, in comparison to healthy control naïve B cells, was determined by Gene Ontology (GO) analysis. Naïve B cells from tumor-bearing mice were uniquely enriched in gene sets associated with EIF2 signaling (for example, Rpl9, Rpl41), iron homeostasis signaling (Hmox1, Tf), EIF4 and p70S6K signaling (Rps3, Rps21), mTOR signaling and complement system (C6, C1qa) (FIG. 2 panel B). The overall expression levels of the gene sets were decreased, suggesting downregulation of protein translation in tumor-educated naïve B cells. Further analysis of the top regulator networks revealed inhibition of IFNG and MYC function. Simultaneous upregulation of several transcriptional regulators of B cell maturation was observed, including pioneer factor Ebf1, Bcl6, Bach2, Pax5, and Spib (Méndez and Mendoza, 2016 PLOS Computational Biology 12:e1004696; Nutt et al. 2015 Nature Reviews Immunology 15:160-171; Tarlinton et al. 2018 Current Opinion in Immunology 20:162-169), as well as markers associated with inflammation and immunosuppressive regulatory B cells (Breg, CD19⁺CD21^{hi}CD1d^{hi}CD5⁺ and IL12a/Ebi3 (IL35) positive B cells) such as STAT1, STAT3, Il12a, Ebi3 and Cd1d (FIG. 2 panel A). Consistent with the initial transcriptional analysis performed in this study and as seen in autoimmune diseases (Wang et al. 2014 Nature Medicine 20:633-641; Kroeger et al. 2016 Clinical Cancer Research 22:3005-3015), these data suggest that specification of immunosuppressive function in B cells may begin at the naïve B cell stage. Concurrent upregulation of markers of maturation and immunosuppression suggest that cancer-associated cues may lead to establishment of B cell dysfunction.

The mechanisms that regulate B cell lineage commitment in malignancy are not known and may have implications for the development of B cell-targeted immunotherapies. It has been demonstrated that IL35+ B cells suppress anti-tumor T cell responses to promote pancreatic tumor growth and restrict efficacy of immunotherapy (Pylayeva-Gupta et al. 2016 Cancer Discovery 6:247-255; Mirlekar et al. 2020 Cancer Immunology Research 8:292-308). In this study, it was observed that IL35 is transcriptionally elevated as early as the naïve B cell stage in mice with PDAC (FIG. 2 panel A). To understand if IL35 may play a cell-autonomous role in subverting B cell development, RNAseq data from naïve B cells in healthy or cancer-bearing mice was analyzed following B cell-specific conditional deletion of IL35 subunit Ebi3 (B^{EBi3-/-}). Loss of EBi3 in B cells resulted in differential expression of 2,753 genes in tumor-educated naïve B cells (FIG. 10 panels B and C). Tumor-educated B^{EBi3-/-} naïve B cells were enriched in gene sets associated with DNA damage-induced 14-3-3σ signaling, cyclins and cell cycle regulation, S-phase entry, GADD45 signaling (Ccnb1, Ccnr1, Cdk1, Ccna2, E2f8), heme biosynthesis (Cpox), and oxidative phosphorylation (MT-CO1, MT-CYB) (FIG. 2 panel C). There was an overall decrease in cellular proliferation capacity. Some of the top regulator networks included tumor suppressor TP53, which was significantly upregulated, possibly accounting for overall reduction in cell cycle progression. Principle component analysis (PCA) indicated tumor-educated naïve B cells as distinct from healthy controls with principal component 1 (PC1) separating populations by disease status (FIG. 2 panel D). Healthy controls from either WT or B^{EBi3-/-} strains had similar gene expression profiles, suggesting that expression of Ebi3 does not perturb steady-state naïve B cell homeostasis. Healthy naïve B cells were also characterized by low levels of HSP70 (Hspa1a and Hspa1b), a regulatory component in B cell-driven suppression in autoimmunity (Wang et al. 2021 J. Molecular Biology 433:166634). Tumor-educated WT naïve B cells were characterized by downregulation of Ppp1ccb, a component of protein synthesis regulators. Loss of Ebi3 in tumor-educated B cells shifted the gene expression profile along both PC1 and PC2 axes, although did not fully recapitulate healthy naïve B cell profiles (FIG. 2 panel D). Loading plot analysis determined a set of genes that were most strongly associated with driving directionality of principal components. Increase in immunoglobulin gene expression (Ighg3, Igh2c, Igkv4) preferentially drove PCA differences in tumor-educated B^{EBi3-/-} naïve B cells as compared to healthy and tumor-educated WT B cells, suggesting initiation of antibody synthesis (FIG. 2 panel D). Indeed, top upregulated molecules included immunoglobulin chains, Il21r and Il4r. Additional analysis showed that tumor-educated B^{EBi3-/-} naïve B cells featured a prominent loss of Breg-associated genes Il12a, Ebi3 and Cd1d (FIG. 2 panel E). In contrast, the plasma cell specifying transcription factors Prdm1 and Xbp1 were significantly elevated in the setting of IL35 loss (FIG. 2 panel E). Thus, in cancer setting, naïve B cells can acquire features associated with cellular stress, translational repression, immunosuppression and maturation, which are partially reversed by IL35 loss with a shift towards plasma cell differentiation features.

Analysis of more terminally differentiated CD21^{hi}CD1d^{hi}CD5⁺ Breg cells confirmed that IL35 could induce significantly higher levels of pSTAT3 and Pax5 (FIG. 2 panel F). Similarly, the intra-tumoral Breg cells isolated from WT mice had higher levels of pSTAT3, Pax5 and BCL6 compared to Bcon cells (FIG. 2 panel G). Consistent with murine data, RNAseq analysis performed on peripheral B cell subsets from PDAC patients revealed significantly increased expression of BACH1, BCL6, STAT1and STAT3 and decreased expression of SDC1 (CD138) gene in CD19⁺CD24^{hi}CD38^{hi} immunoregulatory IL35⁺ B cells, when compared to conventional B cells (FIG. 2 panel H; FIG. 10 panel D). This broad subset of B cells also correlated with diminished cytokine upregulation in T cell activation assay (FIG. 10 panels E and F). Consistent with this, this study found that CD19⁺CD24^{hi}CD38^{hi} B cells isolated from resected splenic tissues of PDAC patients had elevated protein level expression of p35, EBi3, pSTAT3 and Pax5 when compared to CD19⁺CD24^{lo}CD38^{lo} conventional B cells (FIG. 2 panels I, J; n=25). Together, these data provide evidence that PDAC instructs the upregulation of a transcriptional network consisting of pSTAT3, Pax5, and Bcl6.

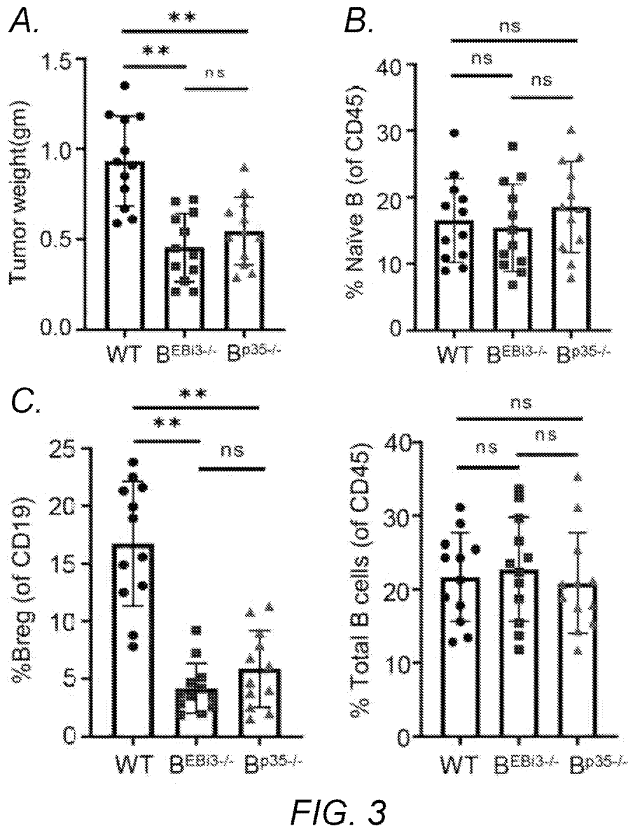

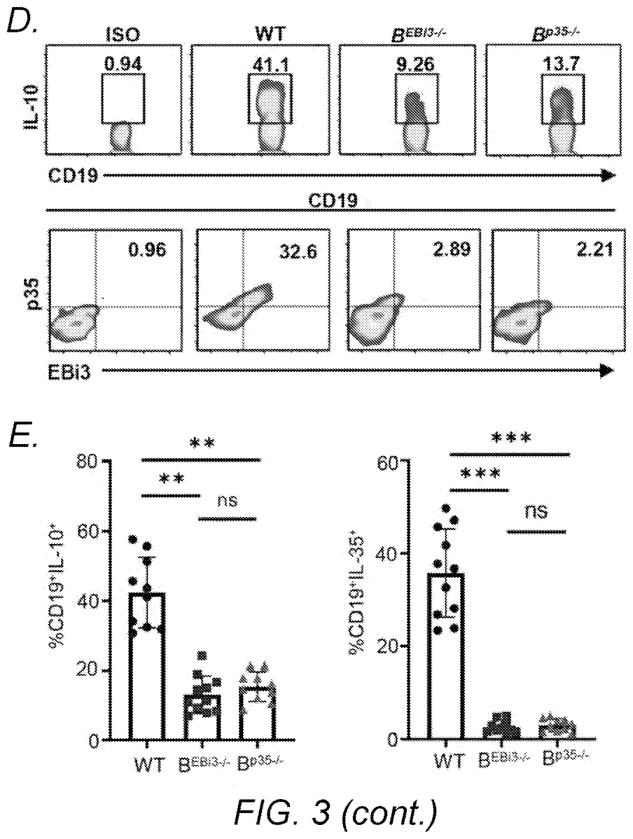

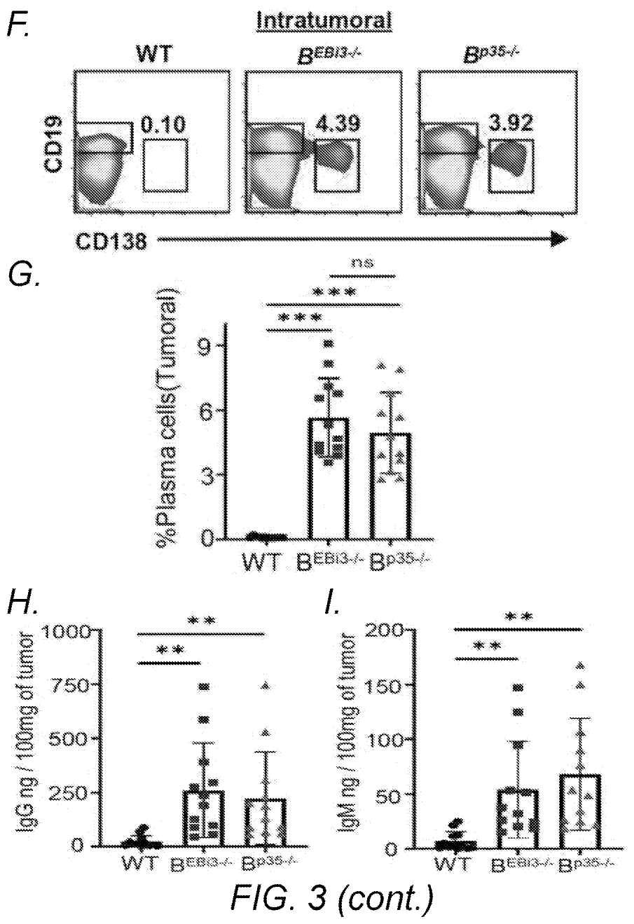

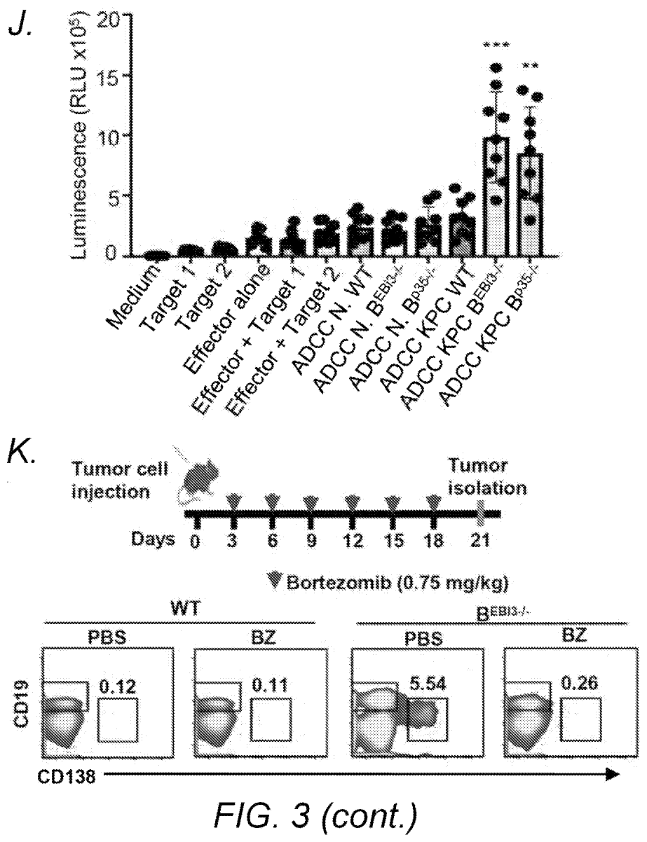

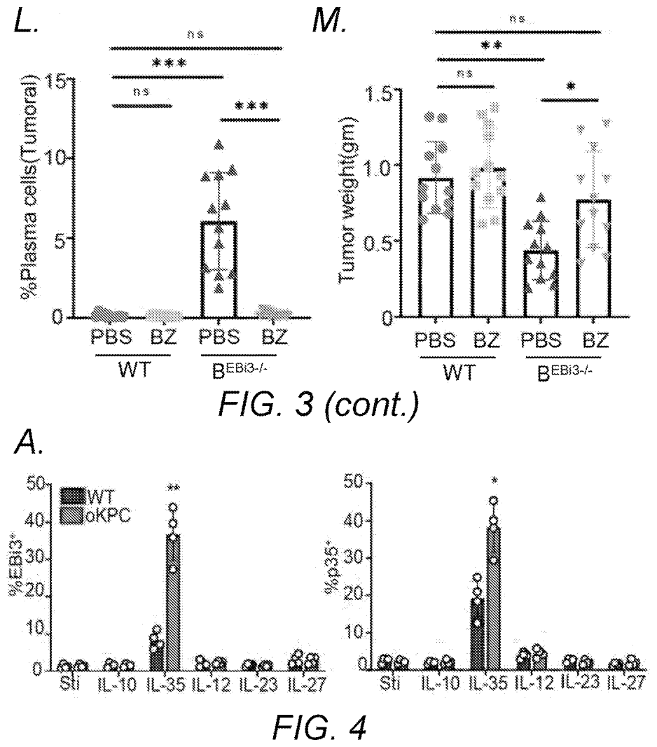

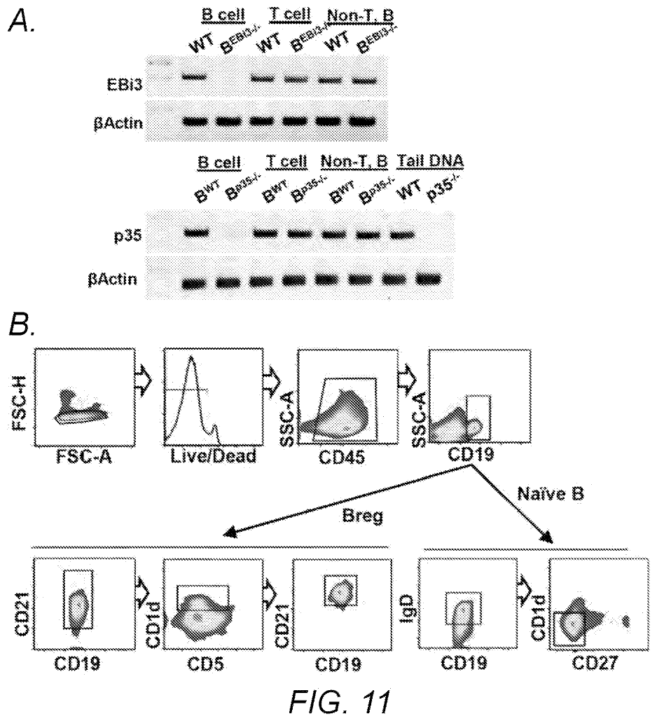

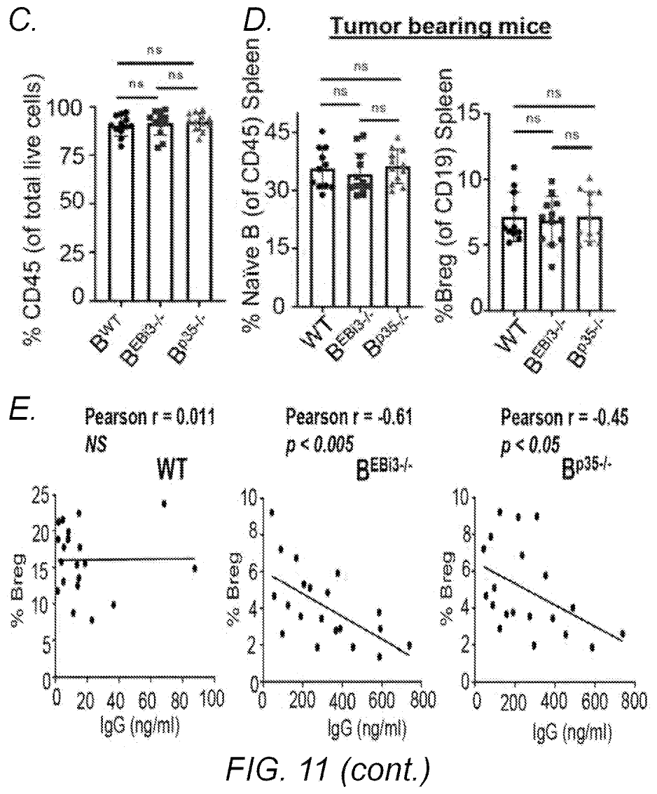

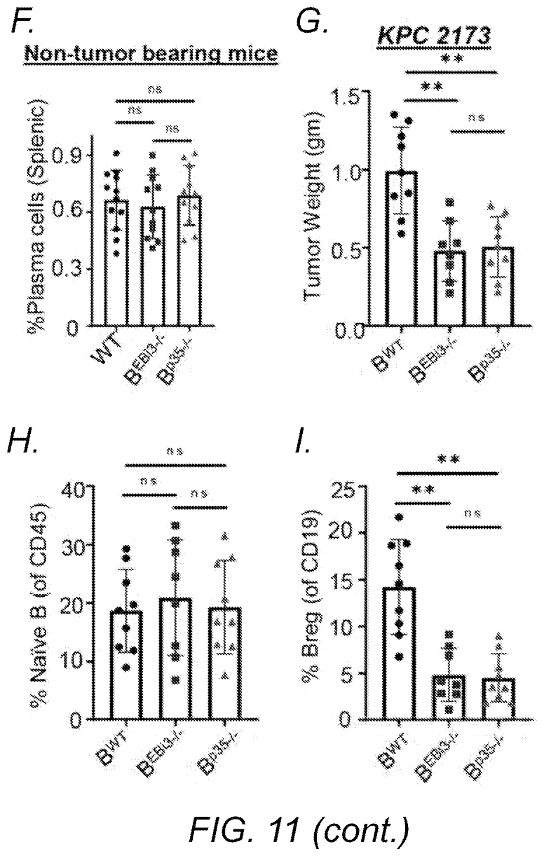

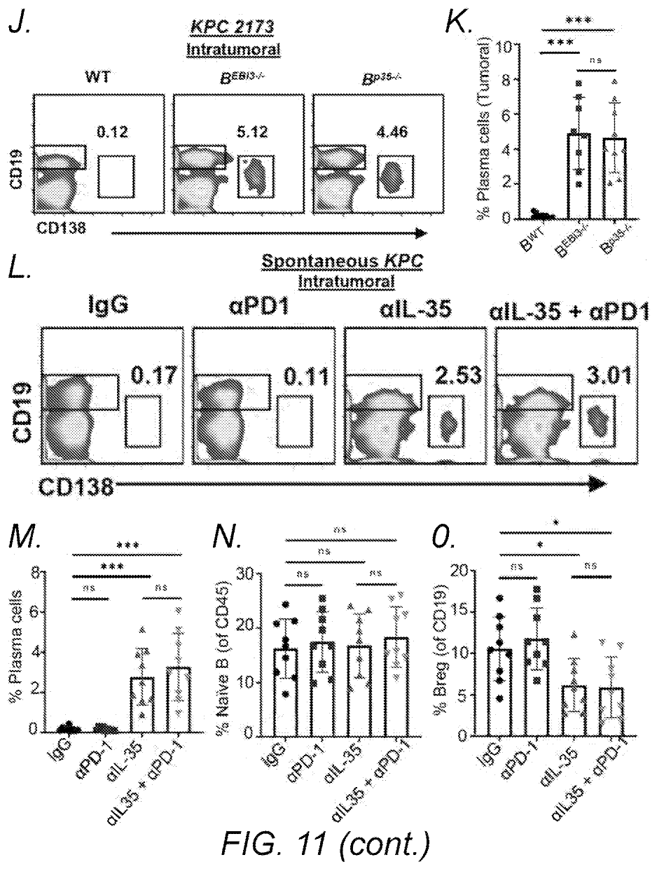

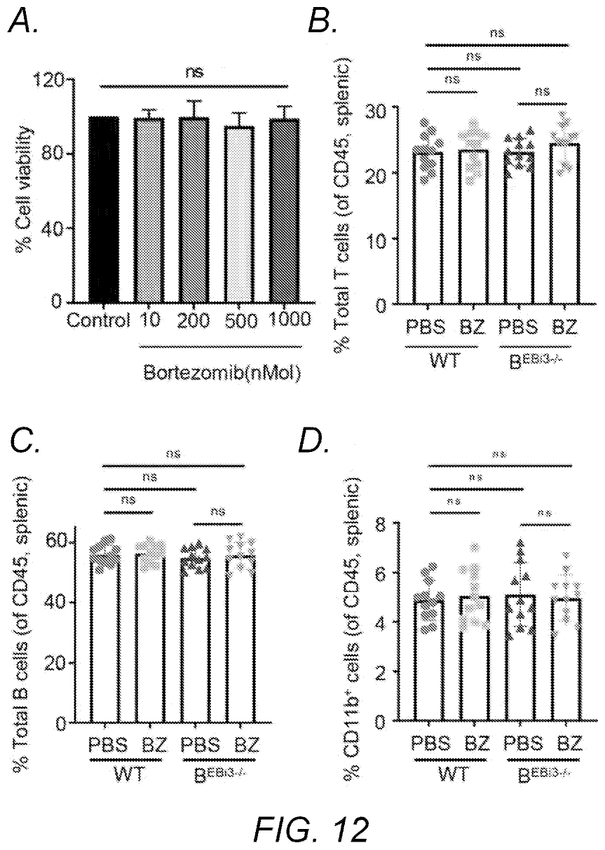

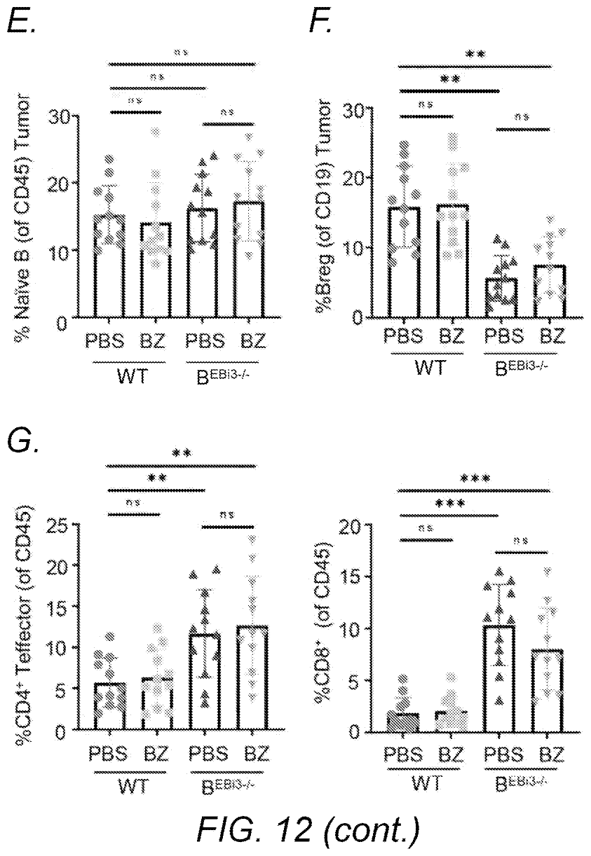

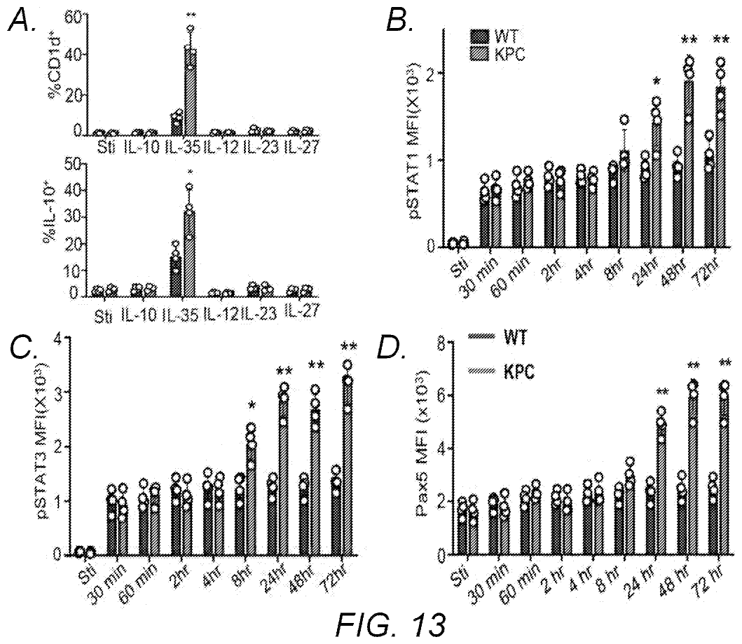

IL35 contributes to B cell dysfunction and suppresses intra-tumoral expansion of plasma cells: B cell subsets in orthotopic tumor-bearing mice with B cell-specific conditional deletion of IL35 subunits p35 or Ebi3 (B^{p35-/-} and B^{EBi3-/-} respectively) were analyzed to understand how cell autonomous IL35 promotes B cell dysfunction in cancer. B cell-specific loss of IL35 resulted in significant reduction in tumor growth (FIG. 3 panel A; FIG. 11 panel A). Analysis of intra-tumoral immune cells revealed that IL35 loss did not significantly alter CD45⁺ leukocyte, total B cell and naïve B cell frequency, but instead specifically decreased the intra-tumoral CD21^{hi}CD1d^{hi}CD5⁺ Breg population (FIG. 3 panels B and C; FIG. 11 panels B-D). Production of immunosuppressive cytokines IL-10 and IL35 by intra-tumoral Breg cells was also significantly reduced as compared to WT counterparts (FIG. 3 panels D and E). On the other hand, consistent with RNAseq data suggesting a shift towards plasma cell differentiation with loss of IL35, there was a significant increase in the intratumoral proportion of plasma cells in mice with B cell specific IL35 deficiency (FIG. 3 panels F and G; FIG. 11 panel E). Immunoglobulin analysis revealed elevated intratumoral IgG and IgM antibody isotypes, which inversely correlated with Breg frequency (FIG. 3 panels H and I). Furthermore, antibody-dependent cellular cytotoxicity assay demonstrated enhanced ability of peripheral effector cells to target tumor cells specifically in the context of IL35 loss, suggesting antigen-specific recognition (FIG. 3 panel J). The frequency of splenic plasma cells in tumor naïve B^{p35-/-} and B^{EBi3-/-} mice remained unchanged, suggesting that IL35 does not regulate plasma cell expansion in a cell intrinsic manner (FIG. 11 panel F). Additional studies using KPC cell line 2173 confirmed that B cell-specific loss of IL35 led increase in accumulation of intra-tumoral plasma cells (FIG. 11 panels G-K). A previous study had shown that treatment with blocking anti-IL35 inhibitor that can deplete rapidly proliferating auto-reactive plasma cells (Alexander et al. 2015 Annals of the Rheumatic Diseases 74:1474-1478; Neubert et al. 2008 Nature Medicine 14:748-755). WT and B^{EBi3-/-} mice were orthotopically injected with KPC cells and treated with bortezomib (0.75 mg/kg) or control (FIG. 3 panel K). Of note, bortezomib did not alter viability of KPC4662 cells in vitro or affect frequency of T cells, total B cells and myeloid cell lineages (FIG. 12 panels A-D). Treatment with bortezomib selectively reduced intra-tumoral plasma cell frequency and rescued tumor growth in B^{EBi3-/-} mice (FIG. 3 panels L and M). As expected, reduced Breg function via loss of Ebi3 led to an increase in intra-tumoral T cell infiltration (FIG. 12 panels E-G). This was not affected by plasma cell depletion, suggesting that IL35 independently affects frequency of effector B and T cells. These data reveal that IL35 contributes to dysfunctional B cell differentiation by both supporting Breg specification and by restricting tumor-reactive plasma cell expansion in PDAC. Thus, B cell effector function may be actively suppressed in PDAC.

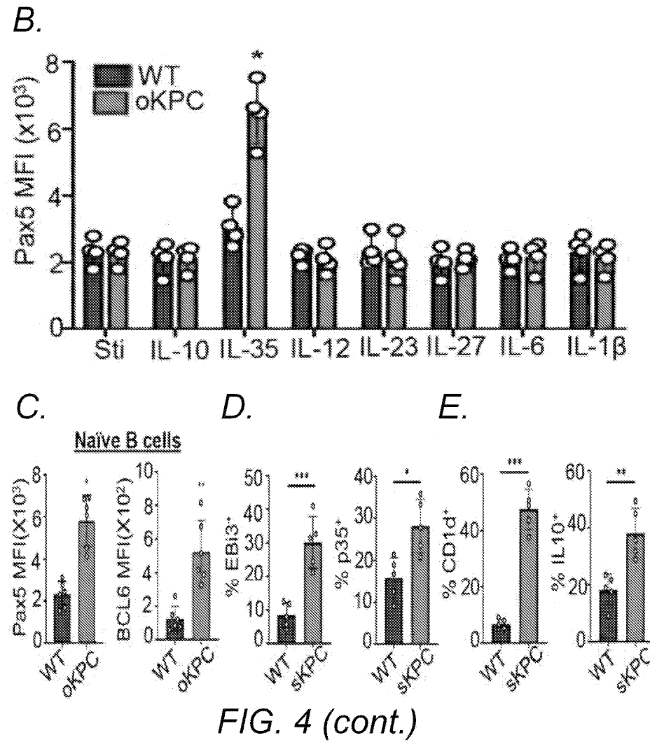

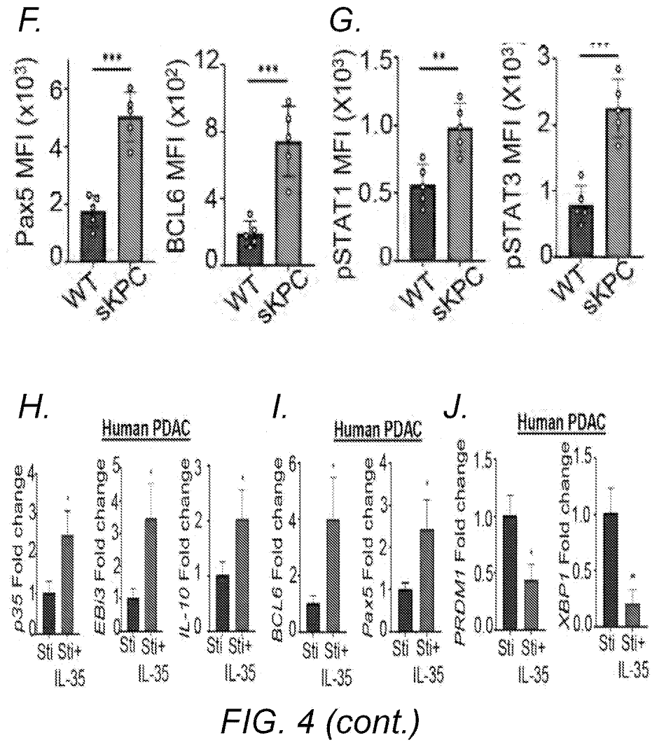

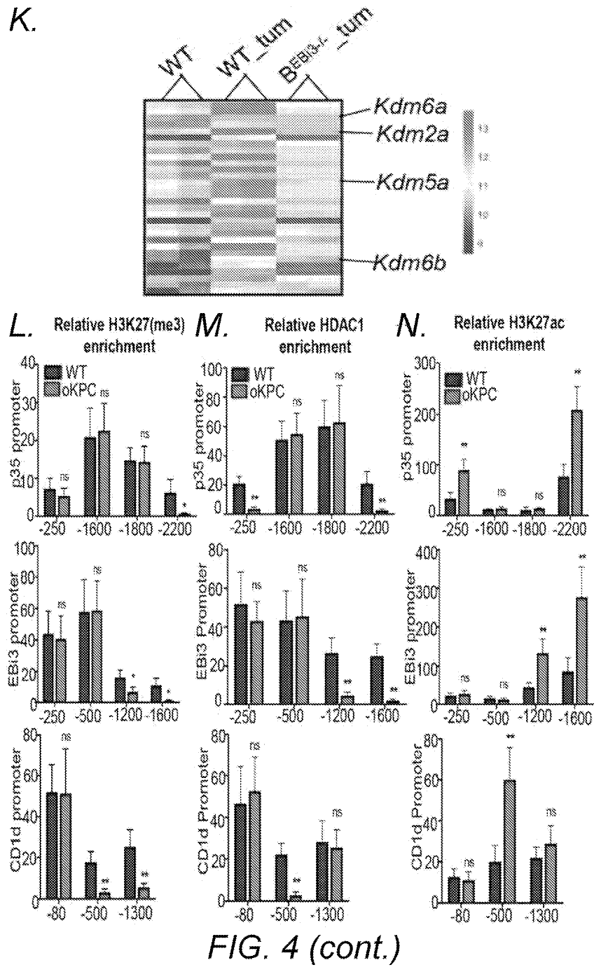

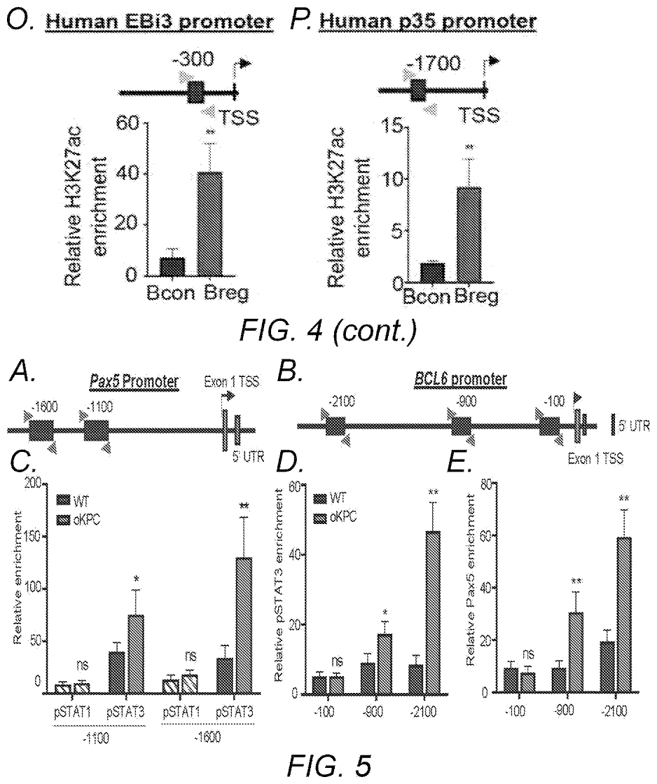

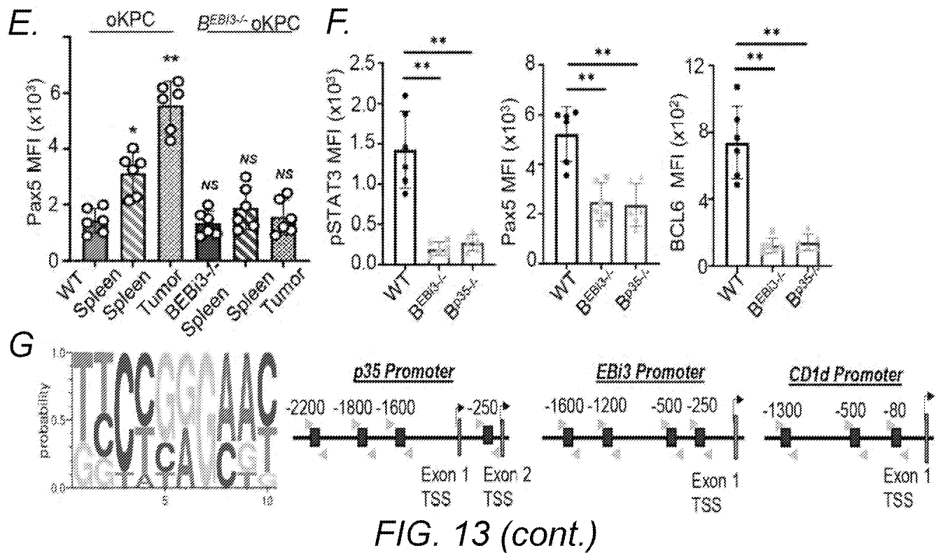

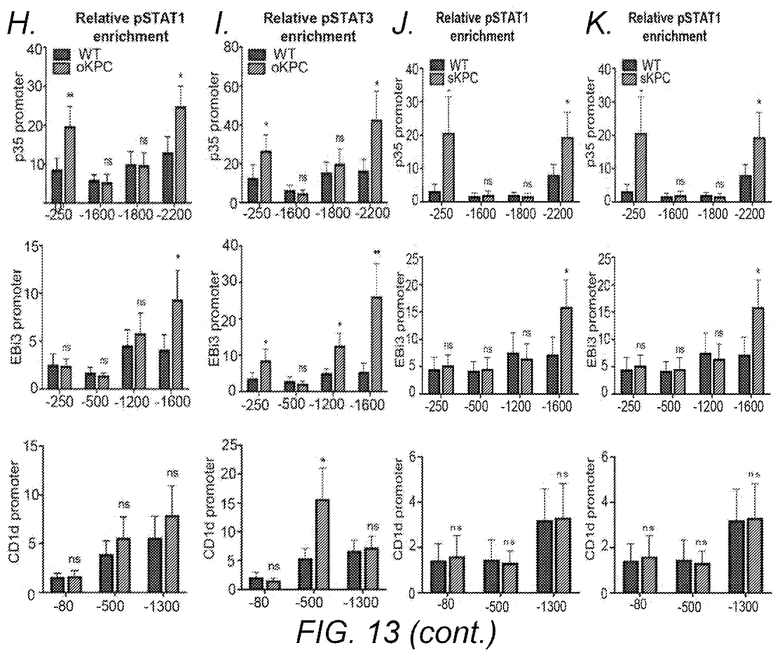

Tumor-educated naïve B cells are primed for dysfunction: To better understand how IL35 may be altering B cell differentiation programs, the differences in response of naïve B cells from healthy or tumor-bearing mice to IL35 stimulation were examined. Treatment of splenic naïve B cells isolated from oKPC mice with LPS/αCD40 and rIL35 induced significantly stronger expression of p35, EBi3, IL10 and CD1d (FIG. 4 panel A; FIG. 13 panel A). This effect was specific to IL35 and was not observed with other tested IL-12 family cytokines or IL-10. Flow cytometry analyses confirmed that regulatory cytokine genes, transcriptional regulators Pax5, BCL6 and activated Stat1 and Stat3 were preferentially enriched in LPS/αCD40/rIL35 treated naïve B cells isolated from oKPC and sKPC mice (FIG. 4 panels B-G; FIG. 13 panels B-D). Patient-derived splenic naïve B cells were also able to respond to rIL35 treatment by increasing expression of p35, EBi3, IL-10, Pax5 and BCL6 and downregulating PRDM1 and XBP1 (FIG. 4 panels H-J). IL35-driven phenotype persisted in Breg cells, as it induced significantly higher levels of Pax5 (FIG. 11 panel E). Similarly, the intra-tumoral Breg cells isolated from WT mice had higher levels of pSTAT3, Pax5 and BCL6 compared to Bcon cells, and this effect was lost with IL35 deficiency (FIG. 13 panel F). Thus, these data reveal that IL35 not only promotes its own expression, but has a broad role in modulating a dysfunctional B cell state characterized by expression of immunosuppressive markers, enrichment of Pax5 and Bcl6 and suppression of plasma cell specifying transcription factors.

The mechanism of transcriptional regulation by IL35 was next investigated. Evaluation of RNAseq data in naïve B cells (FIG. 2), revealed significant changes in expression of chromatin and DNA modifiers that were largely alleviated with IL35 loss (FIG. 4 panel K). In particular, several lysine demethylases were significantly upregulated in tumor-educated naïve B cells. To assess whether treatment with IL35 could modulate chromatin modifications and transcription factor enrichment, in silico analysis of consensus STAT recognition motifs using TFBIND, TFSEARCH and PROMO-ALGGEN (Farre et al. 2003 Nucleic Acids Research 31:3651-3653) was performed, which identified potential binding sites within the p35, EBi3 and Cd1d promoter regions (FIG. 13 panel G). A decrease in repressive mark H3K27 trimethylation, reduced HDAC1 recruitment, and increased H3K27 acetylation at the STAT-binding sites in tumor-educated naïve B cells was observed (FIG. 4 panels L-N). The H3K27ac mark was similarly enriched at STAT3 consensus binding sites within the p35 and EBi3 genes in Breg cells isolated from PDAC patients (FIG. 4 panels O and P). Tumor-educated naïve B cells derived from oKPC or sKPC mice as compared to healthy controls, were significantly more enriched for pSTAT1 and/or pSTAT3 binding at multiple sites within the p35, EBi3, and CD1d gene promoters (FIG. 13 panels H-K). Thus, in tumor-educated B cells, IL35 alters the expression of chromatin regulatory factors and its effects on target loci are associated with significant changes in chromatin modification state.

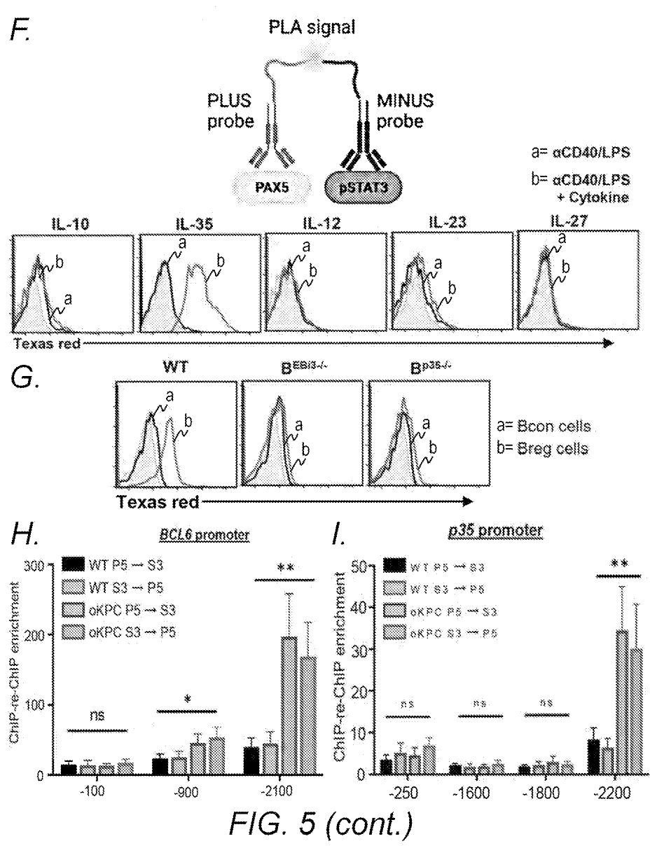

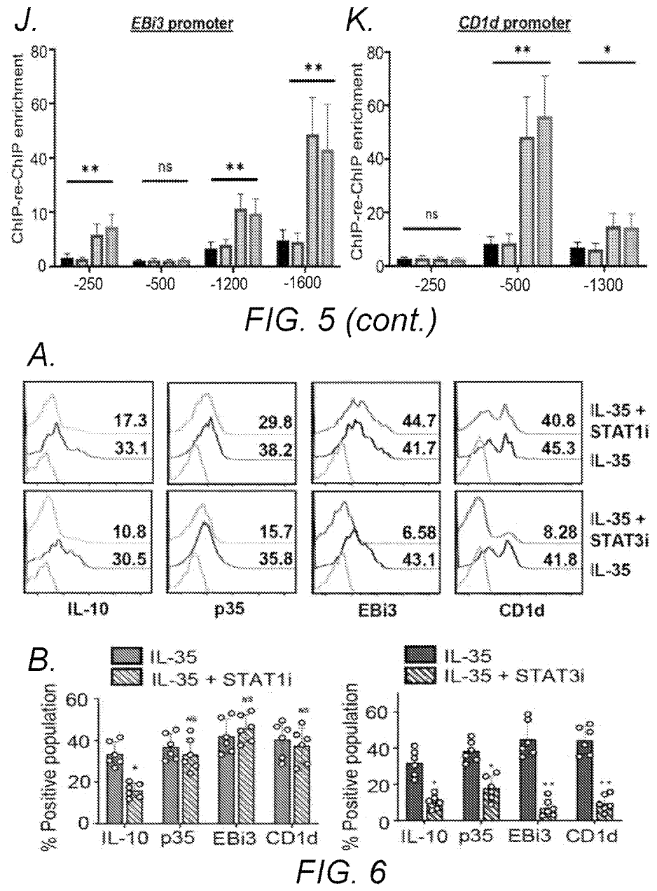

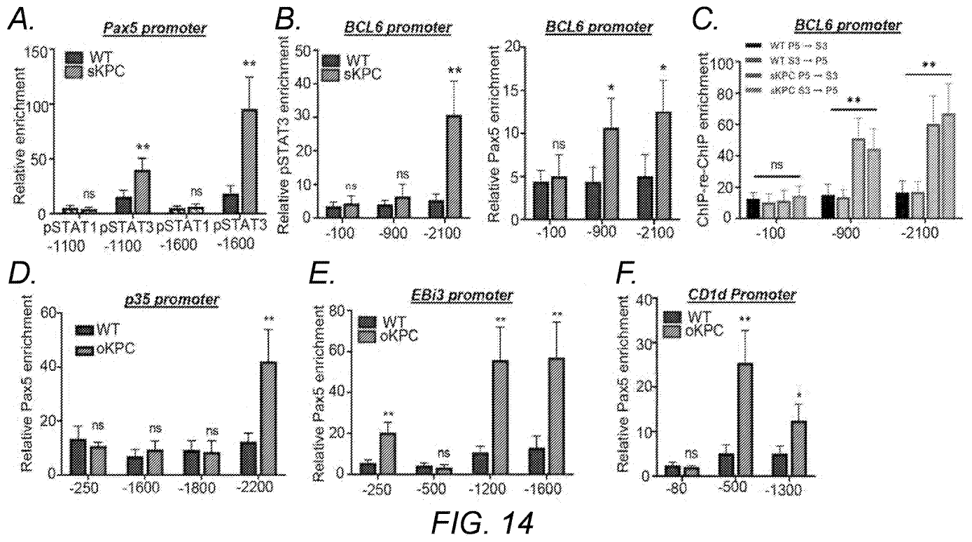

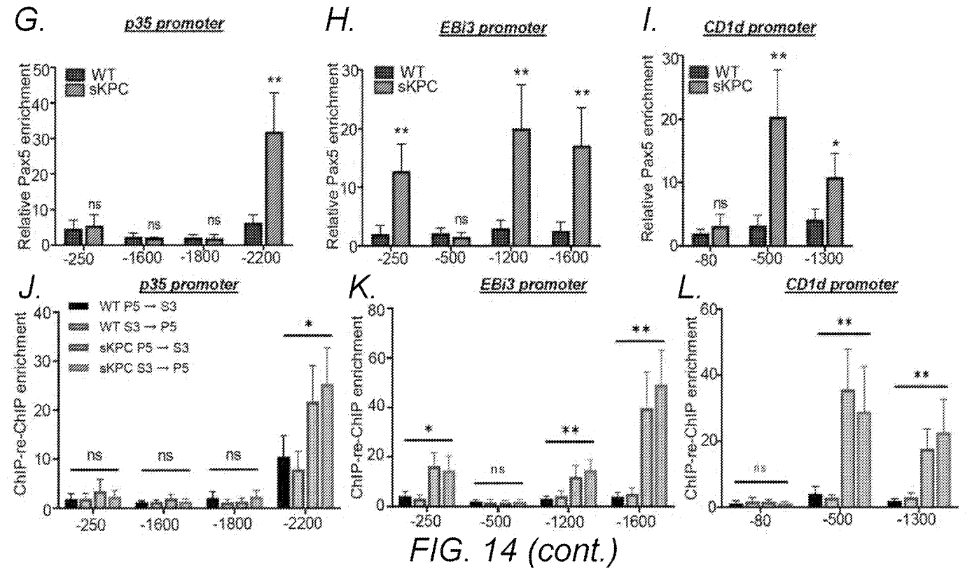

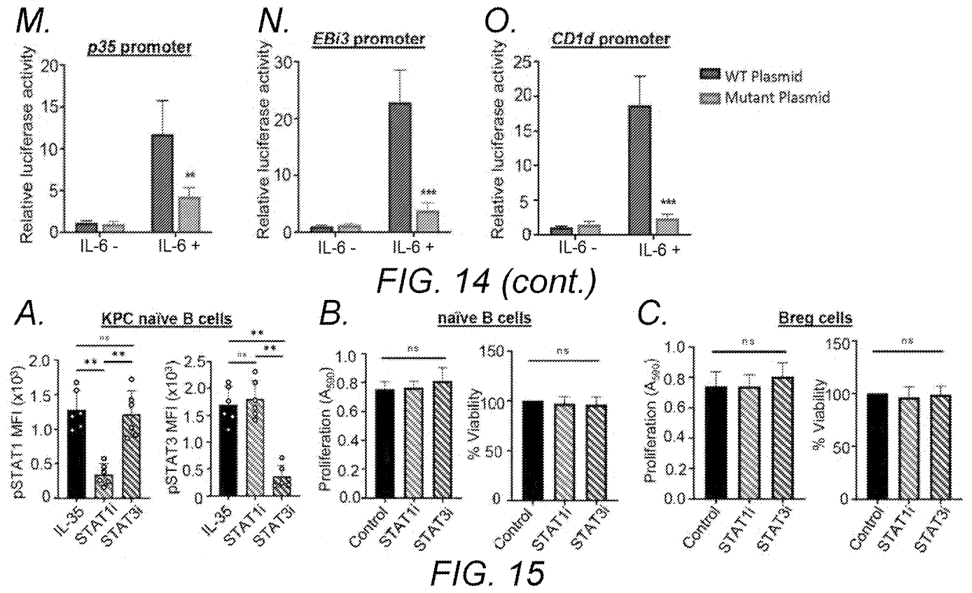

IL35 drives STAT3-Pax5 co-recruitment in tumor-educated B cells: To clarify the mechanism of Pax5 and Bcl6 transcriptional upregulation in tumor-educated naïve B cells, the Pax5 and Bcl6 gene promoters were next analyzed for pSTAT1/3 binding (FIG. 5 panels A and B). Analysis by ChIP showed preferential enrichment of pSTAT3, but not pSTAT1 at the Pax5 promoter region in tumor-educated naïve B cells isolated from oKPC and sKPC mice (FIG. 5 panel C, FIG. 14 panel A). pSTAT3 and Pax5 were also both enriched at Bcl6 promoter in tumor-educated naïve B cells as compared to WT controls (FIG. 5 panels D and E; FIG. 14 panel B). Using Proximity Ligation Assay (PLA), it was discovered that rIL35, but not rIL-10, rIL-12, rIL-23 and rIL-27, could specifically induce interaction of STAT3 and Pax5 in tumor-educated naïve B cells (FIG. 5 panel F). Pax5 and STAT3 interaction was lost upon B cell specific deletion of IL35 (FIG. 5 panel G). ChIP-re-ChIP using double pull-down with pSTAT3 and Pax5 antibodies also confirmed that the STAT3-Pax5 complex binds to the Bcl6 gene promoter (FIG. 5 panel H, FIG. 14 panel C). Pax5 had a similar binding pattern to pSTAT3 in naïve B cells within the gene promoter regions of p35, EBi3 and CD1d and exposure to rIL35 strongly favored enrichment of Pax5 in tumor-educated B cells from oKPC and sKPC mice (IG. 13 panels D-I). Indeed, significant enrichment for co-recruitment of STAT3/Pax5 complex was observed at the promoters of regulatory genes in tumor-educated naïve B cells (FIG. 5 panels I-K; FIG. 14 panels J-L). The STAT3-Pax5 binding sites were important for driving transcription of regulatory genes, as deletion of STAT3-Pax5 binding sequences at sites −2200 (p35), −1600 (EBi3) and −500 (Cd1d) resulted in decreased luciferase activity in 293T cells upon STAT3 activation (FIG. 14 panels M-O). These results indicate that formation of a pSTAT3-Pax5 complex is a specific mechanism for transcriptional modulation by IL35 and may underlie the transcriptional dysregulation of B cell differentiation in PDAC.

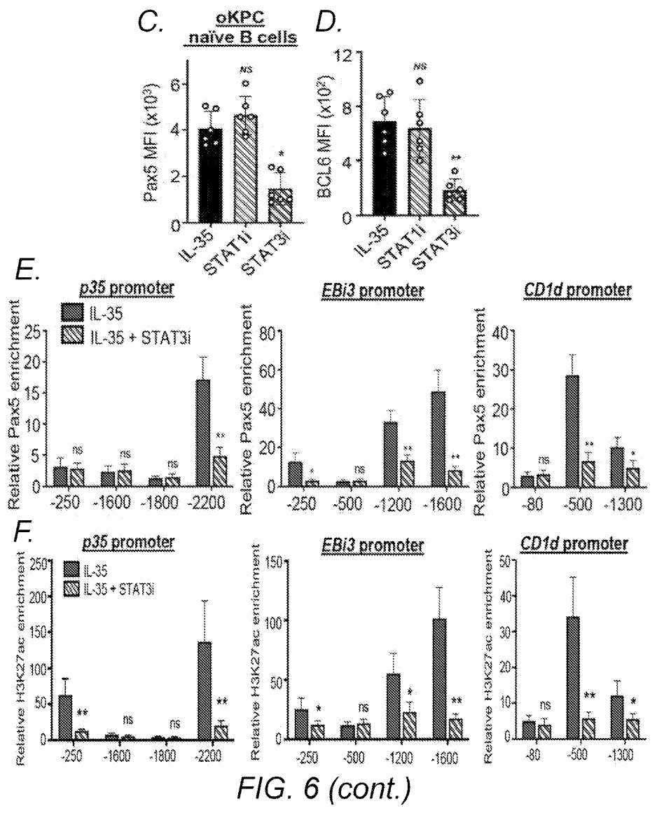

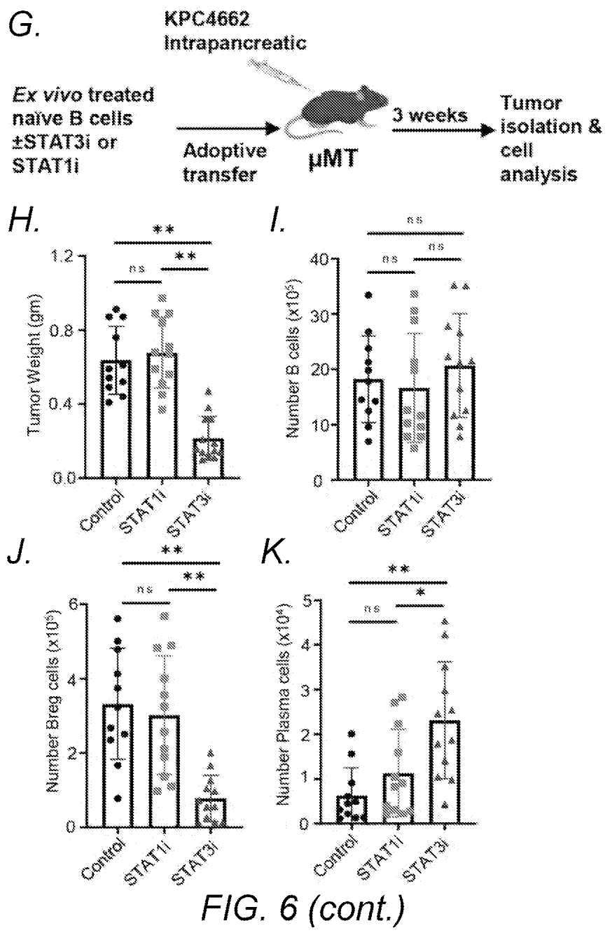

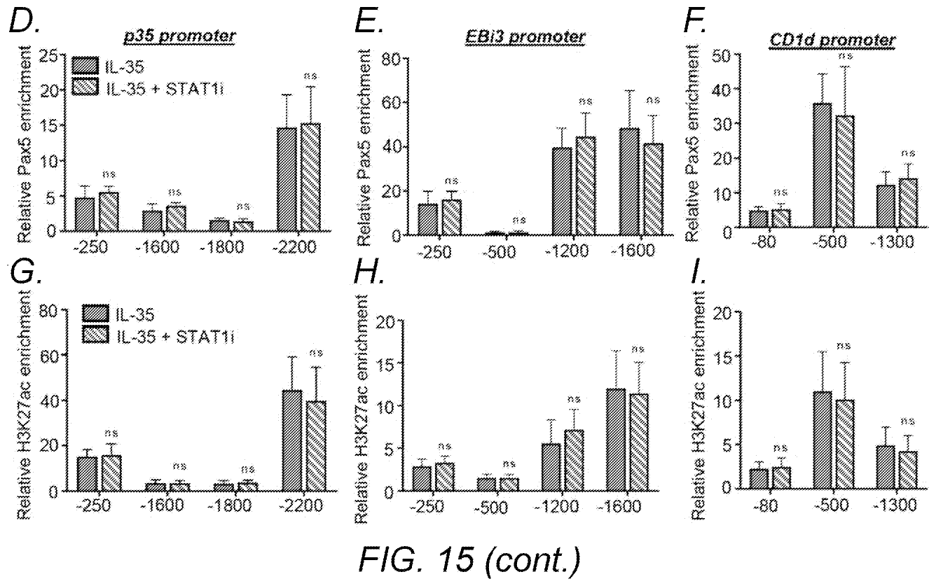

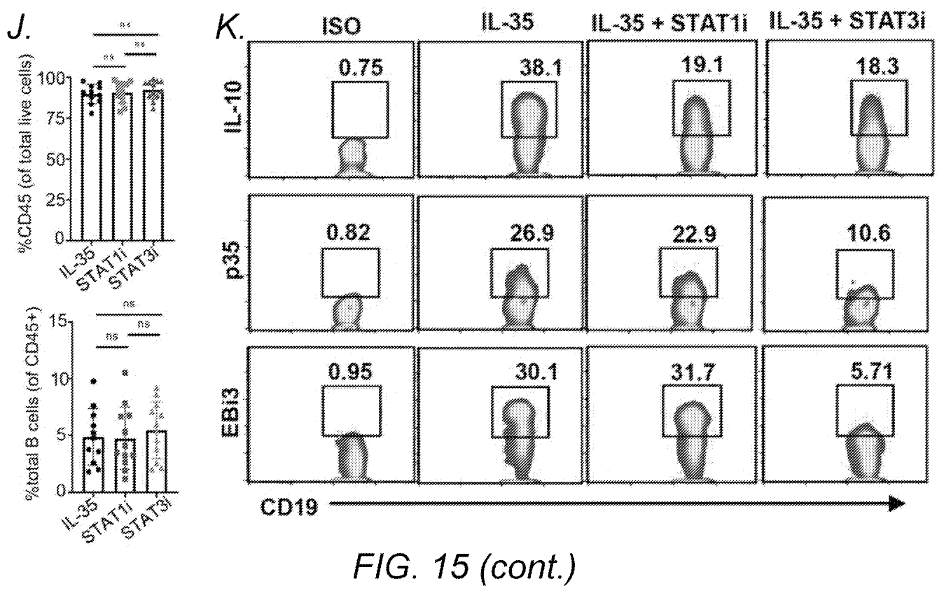

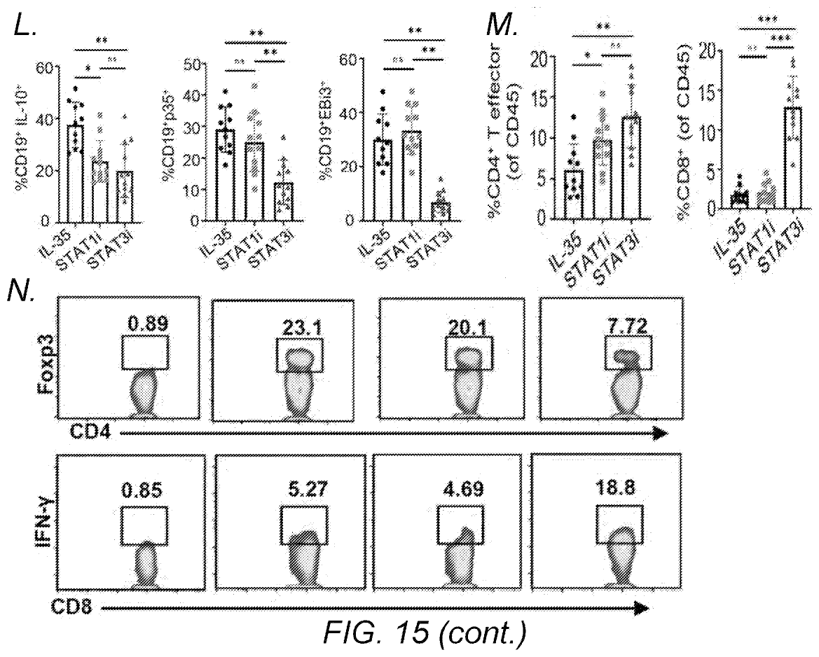

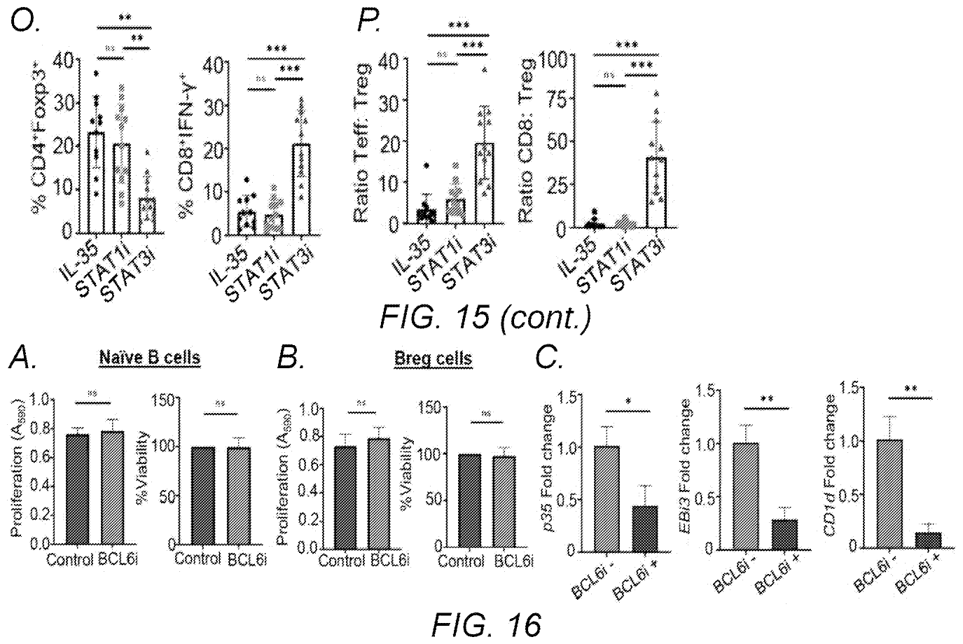

IL35-STAT3 axis deregulates naive B cells in pancreatic cancer: To examine the functional consequences of STAT1/3 regulation on PDAC-associated B cell function, tumor-educated splenic naïve B cells were treated with LPS/αCD40/rIL35 and a STAT1 or STAT3 inhibitor (Fludarabine and STA-21, respectively) (FIG. 15 panel A). The STAT1 and STAT3 inhibitor did not alter proliferation or viability of naïve or Breg cells (FIG. 15 panels B and C). Inhibition of STAT3, but not STAT1 significantly reduced expression of p35, EBi3, CD1d, Pax5 and Bcl6, whereas IL10 was regulated by both STAT1 or STAT3, suggesting that IL35/STAT3 exerts a dominant role in specifying IL35+ Breg cell fate (FIG. 6 panels A-D). Furthermore, inhibition of STAT3, but not STAT1 reduced acetylation levels and Pax5 recruitment at p35, EBi3 and Cd1d gene promoters in tumor-educated naïve B cells, indicating that STAT3 is required for IL35 mediated increases in chromatin modification and Pax5 recruitment at these regulatory loci (FIG. 6 panels E and F; FIG. 15 panels D-I).

To determine whether STAT3 was required for naïve B cell dysfunction in PDAC, naïve B cells from tumor-bearing mice were treated with LPS/αCD40/rIL35 and a STAT1 or STAT3 inhibitor and adoptively transferred to B cell deficient μMT mice (FIG. 6 panel G). Three weeks post-orthotopic injection of KPC cells, inhibition of STAT3 but not STAT1 in B cells significantly reduced tumor burden, decreased Breg frequency and cytokine production, and enhanced intratumoral accumulation of plasma cells (FIG. 6 panels H-K, FIG. 15 panels J-L). Inhibition of STAT3 in B cells also reduced intra-tumoral Treg frequency and enhanced intra-tumoral activity of CD4⁺ effector cells and cytotoxic CD8⁺ T cells (FIG. 14 panels M-P). These data indicate that STAT3 is essential for maintaining immunosuppression of naïve B cells in PDAC, and that targeting STAT3 is sufficient to induce anti-tumor plasma B cells.

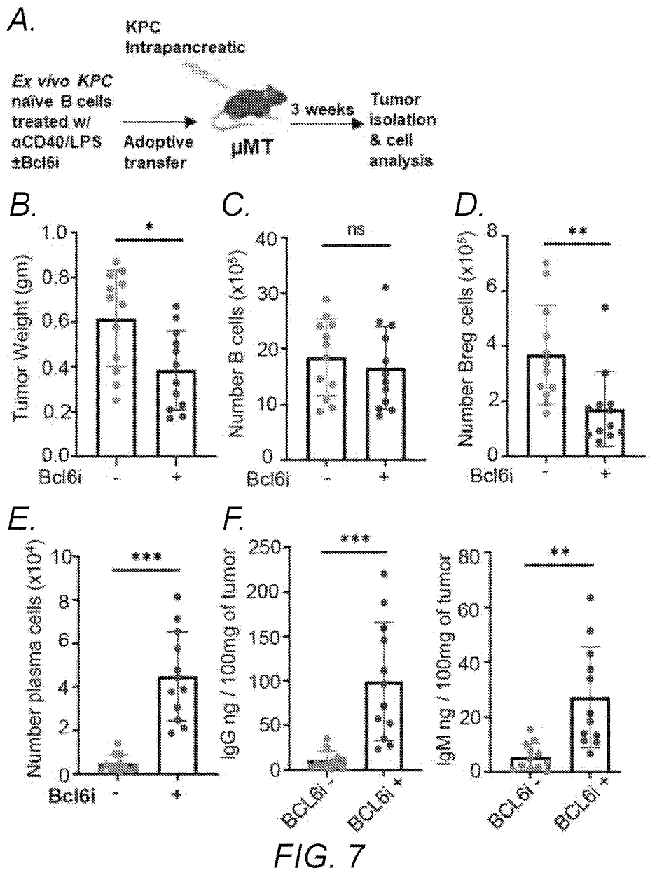

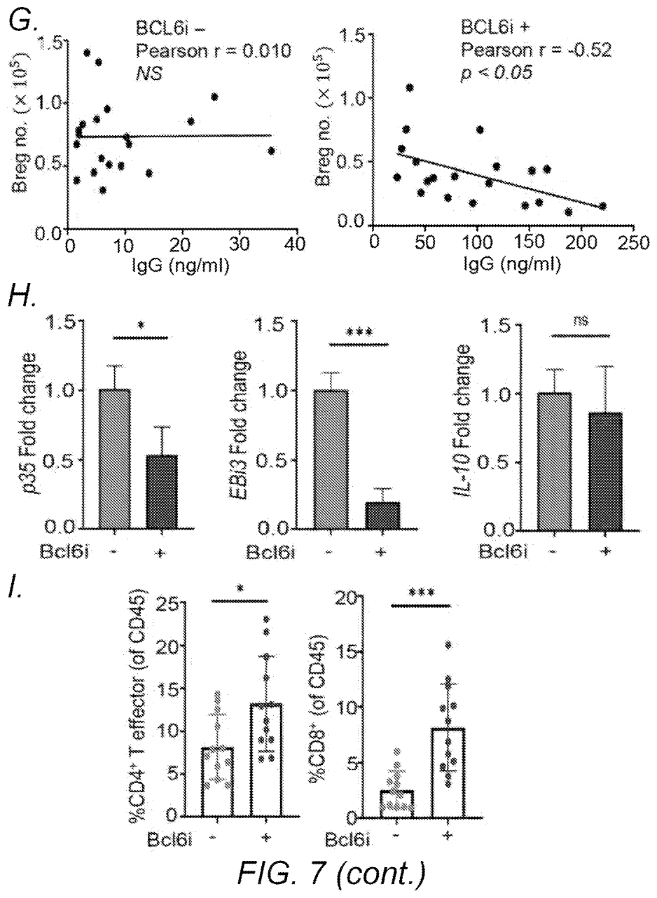

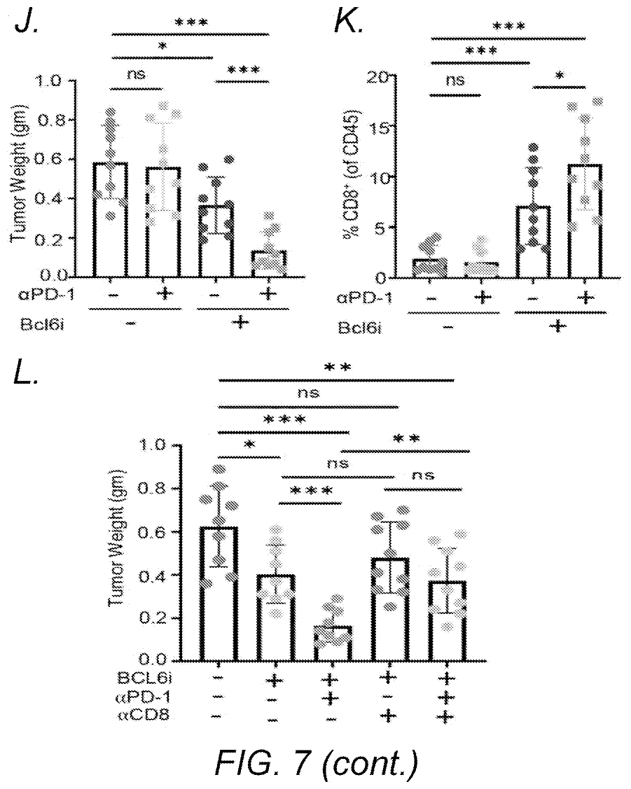

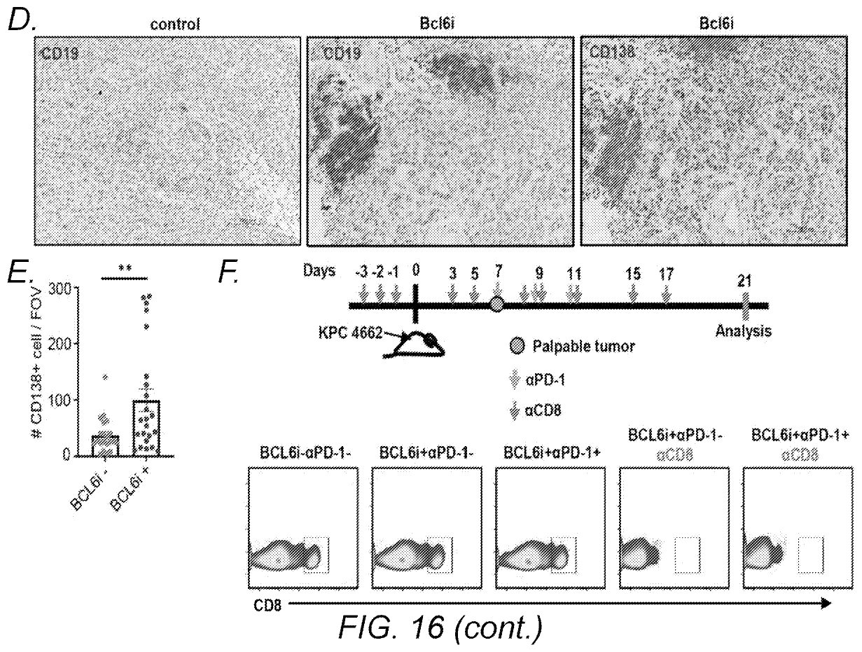

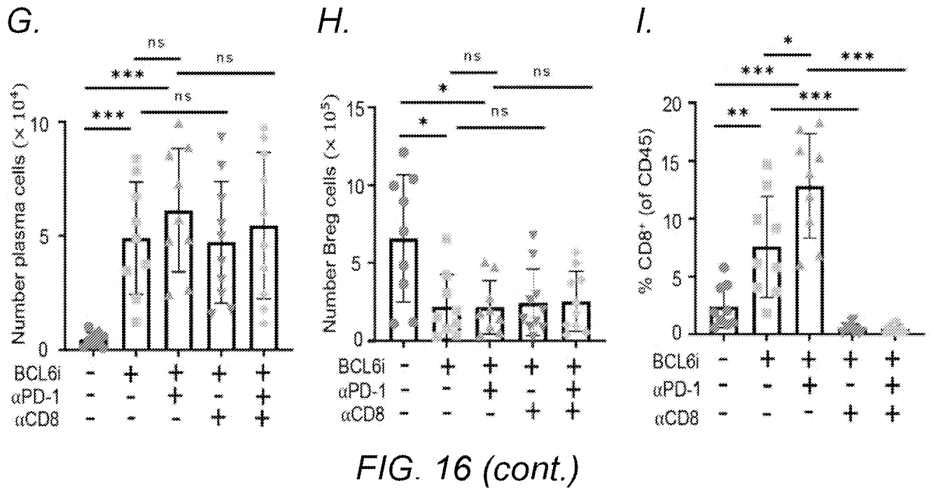

BCL6 expression in tumor educated naïve B cells is required to maintain Breg/plasma cell balance: Tumor-educated activated naïve B cells were treated with the Bcl6 inhibitor 79-6 (Bcl6i)(FIG. 7 panel A) to examine how upregulation of the transcription factor Bcl6 controls B cell differentiation in PDAC. Treatment with the Bcl6 inhibitor did not alter proliferation and viability of naïve or Breg cells (FIG. 16 panels A and B). Adoptive transfer of Bcl6i-treated naïve B cells to μMT mice resulted in inhibition of tumor growth, accompanied by decreases in intra-tumoral Breg frequency and cytokine production, as well as enhanced intra-tumoral accumulation of plasma cells (FIG. 7 panels B-E, FIG. 16 panels C-E). Increased intra-tumoral IgG and IgM concentration was detected, which inversely correlated with Breg frequency (FIG. 7 panels F and G). Furthermore, inhibition of BCL6 resulted in reduced expression of p35 and EBi3 in intra-tumoral Bregs but did not affect the expression of IL-10, demonstrating that BCL6 could potentiate IL35+ Breg cell fate (FIG. 7 panel H). A significant increase in the frequency of intra-tumoral CD4+ and CD8+ T cells was also observed, likely due to reduction in regulatory B cell function (FIG. 7 panel I).

Recent studies have suggested that improved B and T cell function are a prerequisite for efficacy of immune checkpoint blockade (Hollern et al. 2019 Cell 179:1191-1206). In this study, it was found that treatment of PDAC tumor-bearing animals with anti-PD1 in combination with Bcl6 blockade in naïve B cells led to an increased frequency of intra-tumoral CD8⁺ T cells and significantly reduced tumor growth (FIG. 7 panels J and K). Depletion of CD8⁺ T partially rescued tumor growth and did not alter plasma cell frequency, suggesting that T cell and B cell-directed targeting of the tumor may be additive (FIG. 7 panel L; FIG. 16 panels F-I). These results demonstrate that adoptive transfer of tumor-educated naïve B cells after BCL6 inhibition is sufficient to reprogram B cell-mediated anti-tumor immune responses and overcome resistance to anti-PD1 immune checkpoint inhibitor therapy in PDAC.

In summary, data from this study indicates that B cells can be derailed from their normal effector function at the naïve B cell stage, even in the presence of tumor-specific antigens. This study demonstrates that B cell dysfunction in cancer may be an outcome of an active suppression program (mediated by IL35 as this example) that occurs during tumorigenesis. Specifically, IL35/STAT3 signaling axis may shift naïve B cells away from plasma cell differentiation and towards regulatory function by stabilizing interaction between pioneer factor Pax5 and pStat3. Genetic or pharmacologic inhibition of the IL35/STAT3/Bcl6 signaling axis may promote intra-tumoral accumulation of plasma B cells, impacting tumor growth and resistance to immunotherapy, and transcriptional reprogramming of naïve B cells in PDAC may be therapeutic.

### Example 2: Methods as Used to Example 1

Bulk murine naïve B cell RNA sequencing data has been submitted to the NIH Gene Expression Omnibus (GEO) repository and is available under accession GSE179797.

Animals were maintained in a specific pathogen-free facility. Six-to-eight-week-old wild-type (WT) C57Bl/6J mice were purchased from The Charles River Laboratories (stock #027). Six- to eight-week-old, Il10−/− (stock #002251), p35−/− (stock #002692), Ebi3−/− (stock #008691) and μMT (stock #002288) mouse strains were purchased from The Jackson Laboratory. Both male and female mice were used for orthotopic injections of PDAC cells. The KrasLSL-G12D/+; Trp53LSL-R172H/+; p48Cre/+(KPC) mice and Ebi3Tom.L/L mice have been described previously (Hingorani et al. 2005 Cancer Cell 7:469-483; Turnis et al. 2016 Immunity 44:316-329). CD19Cre; Ebi3L/L mice were generated by crossing CD19Cre mice 78 to Ebi3Tom.L/L mice for two generations to obtain homozygosity at Ebi3 locus. Resulting mice lack expression of Ebi3 in B cells (BEbi3−/−). CD19Cre; Ebi3+/− littermates were used as controls. Unless otherwise indicated, experiments were performed using 7-8-week-old mice of indicated genotypes with at least 6-12 mice per group in triplicate.

The murine PDAC cell line KPC4662 and KPC2173 were derived from primary pancreatic tumors of C57Bl/6J KPC mice (Bayne et al. 2012 Cancer Cell 21:822-835). GFP-labeled KPC cells were generated as described previously (Pylayeva-Gupta et al. 2016 Cancer Discovery 6:247-255). Cells were maintained at 37 °C and 5% CO2 in complete DMEM (#11995-065, Gibco, 10% FCS and 1% penicillin-streptomycin #15140-122, Gibco) and were confirmed to be Mycoplasma and endotoxin free. Cells were used at <16 passages.

Informed consent was obtained from the patients and healthy donors before blood donation for human samples. Samples analyzed included splenic immune cells isolated from PDAC patients, where human resected spleen samples were collected from 25 patients with pancreatic ductal adenocarcinoma, and tumor microarray containing normal adjacent and PDAC tumor samples. All samples were received as de-identified, therefore, the information on the age and/or gender of the donors is not available.

Primary mouse or human lymphocytes, including naïve B cells, regulatory B cells (Breg), conventional B cells (Bcon) and T cells were isolated and maintained in complete RPMI media containing 10% FCS with 1× penicillin-streptomycin (#15140-122, Gibco) antibiotics for 24-72 hr. Details of specific culture conditions is described below.

Sorted naïve B, Breg and Bcon cells were activated with 1 μg/ml αCD40, 2 μg/ml LPS and/or rIL35 (50 ng/ml), rIL-12 (20 ng/ml), rIL-10 (20 ng/ml), rIL-23 (20 ng/ml) and rIL-27 (20 ng/ml) as indicated. Naïve B cells were cultured for 72 hrs, while Breg and Bcon cells were cultured for 48 hrs at 370 C and 5% CO2. Sorted T cells were stimulated with 1 μg/ml αCD3 and 2 μg/ml αCD28 for 48 hrs prior to PMA/Ionomycin stimulation. For in vitro CD8+ T-cell culture, splenic CD8+ T cells specific for the OVA257-264 (InvivoGen) antigen were sorted (>98% purity) from WT mice immunized with OVA257-264 for 1 week (10 μg/mouse). T cells were cultured with plate bound αCD3 (1 μg/mL, Bio X Cell) and soluble αCD28 (2 μg/mL, BioXCell), for 48 hrs.

For bone marrow mouse chimera generation, BWT and Bp35−/− mice were obtained by a mixed bone marrow chimera method. BWT and Bp35−/− mice were obtained by a mixed bone marrow chimera method using lethally irradiated (1,000 cGy radiation delivered from cesium source) using C57BL/6J mice as recipients. Recipients were reconstituted with a mixture of bone marrow cells from B cell-deficient μMT mice (The Jackson Laboratory, #002288) or WT C57BL/6J mice (80%), respectively, and p35−/− mice (20%; The Jackson Laboratory, #002692). A total of 10×106 bone marrow cells was injected intravenously into the irradiated WT recipients. The chimeric animals were used after eight weeks and specific deletion of p35 gene in B cells was confirmed by PCR.

For intrapancreatic injection of cancer cells, mice were anesthetized using a ketamine (100 mg/kg)/xylazine (10 mg/kg; Med-Vet International) cocktail. The depth of anesthesia was confirmed by verifying an absence of response to toe pinch. An incision in the left flank was made, and 75,000 KPC cells in ice-cold PBS mixed at 1:1 dilution with Matrigel (#354234, Corning) in a volume of 50 μL were injected using a 28-gauge needle into the tail of the pancreas. The wound was closed in two layers, and the animals were given the pain reliever buprenorphine (0.1 mg/kg; Med-Vet International) once subcutaneously after orthotopic surgery. To analyze the functional effects of plasma cells, we treated mice intravenously with 0.75 mg/kg body weight bortezomib (Millipore-sigma) twice weekly and control mice with equivalent volume of solvent PBS for 3 weeks. After 3 weeks, mice were sacrificed for tumor analysis.

In mouse tumor treatment studies, for therapeutic treatment with immune checkpoint blockade, anti-PD-1 (RMP1-14, Bio X Cell) or their respective IgG isotype controls were injected at 200 μg/injection on days 7, 9, and 11, once an orthotopic tumor reached 4 to 5 mm (day 7). Three doses of antibody were given in total, on days 7, 9, and 11 after injection of KPC cells and mice were sacrificed after 3 weeks for tumor analysis.

For lymphocyte isolation, single-cell suspensions were prepared from tumors and spleens isolated from orthotopic and/or adoptive transfer models. Spleens were mechanically disrupted using the plunger end of a 5 mL syringe and re-suspended in 1% FBS/PBS. Spleen samples were processed following RBC lysis (eBioscience; 00-4333-57). For isolation of tumor-infiltrating lymphocytes, tumor tissue was minced into 1 to 2 mm pieces and digested with collagenase IV (1.25 mg/mL; #LS004188, Worthington), 0.1% soybean trypsin inhibitor (#T9128, Sigma), hyaluronidase (1 mg/mL; #LS002592, Worthington), and DNase I (100 μg/mL; #LS002007, Worthington) in complete DMEM for 30 minutes at 370 C. Cell suspensions were passed through a 70-μm cell strainer (Falcon) and resuspended in RPMI media (Gibco). Lymphocytes were isolated from processed tumor tissues by OptiPrep (Sigma) density gradient centrifugation. MACS isolation of total CD45+ leukocytes (MACS Miltenyi Biotec #130-052-301) was performed on the leukocyte-enriched fraction according to Miltenyi Biotec protocol, and the purity was >90%. Cells were stained with fluorophore-labeled antibodies for 30 minutes on ice in FACS buffer (PBS with 3% FCS and 0.05% sodium azide). After staining, cells were washed twice with FACS buffer and resuspended in sorting buffer (PBS with 1% FCS and 0.05% sodium azide). Cell sorting using a BD FACS ARIA III sorter was performed to isolate CD19+IgDhiCD1d−CD27− naïve B cells, CD19+CD21hiCD5+CD1dhi regulatory B cells (Breg), CD19+CD21loCD5−CD1d− conventional B cells (Bcon), CD4+ and CD8+ T cells. Cells were collected in complete RPMI media containing 10% FCS with 1× penicillin-streptomycin (#15140-122, Gibco) antibiotics. More than 97% purity was achieved.

For Breg and Bcon cell isolation from human spleen, spleen samples were processed as described above by mechanically disrupting followed by RBC lysis. The isolated splenocytes were then stained with anti-human CD19 (HIB19; BioLegend), CD24 (ML5; BioLegend), and CD38 (HB-7; BioLegend) in FACS buffer for 20 minutes on ice. CD19+CD24hiCD38 hi Bregs and CD19+CD24loCD38lo Bcon cells were sorted using a BD FACS ARIAIII, and cells were collected in complete RPMI media. More than 97% cell purity was achieved.

The viability of KPC 4662 cells with 10, 200, 500 and 1000 nMol of bortezomib were assessed with MTT (Sigma #M5655) as per manufacturer instructions. Briefly, the 10 μL from 5 mg/mL MTT stock was added in each well of a 96 well plate and incubated at 37 °C for 3 hours. After incubation, 150 μl of DMSO were added in each well and plate was kept on orbital shaker for 15 min and was read within 1 hr at 590 nm.

For B cell T cell co-culture experiments, mouse splenic Bregs (CD19+CD21hiCD5+CD1Dhi) were sorted by flow cytometry from spleens of WT, KPC, and tumor-bearing 110−/−, p35−/− and EBi3−/− mice (>97% purity), as described above. A total of 100,000 Bregs or Bcon cells and 100,000 CD4+ or CD8+ T cells (1:1 ratio) were co-cultured in the 96-well Transwell plates, with B cells occupying the top chamber and CD4+ or CD8+ T cells the bottom chamber (Corning; 3381) for 48 hrs. B cells were activated by αCD40 (1 μg/mL, eBioscience) and LPS (2 μg/mL, Sigma) for 48 hrs, and T cells were activated by plate bound αCD3 (1 μg/mL) and soluble αCD28 (2 μg/mL). Cytokine secretion of T cells was evaluated by flow cytometry, as described below. For co-culture of B cells with T cells from PDAC patients, splenic CD19+CD24hiCD38 hi Bregs and CD19+CD24loCD38lo Bcon cells were sorted by flow cytometry (>97% purity), as described above. The Breg or Bcon cells were co-cultured with CD4+ or CD8+ T cells in 1:1 ratio and activated as described above and the expression of effector cytokines from T cells was evaluated by qPCR analysis of gene expression, as described below.

For in vitro treatment and adoptive transfer of B cells experiments, naïve B cells (CD19+IgDhiCD1d−CD27−) were isolated from spleens of KPC mice by BD FACS-ARIA III flow cytometry sorting (purity >98%). Sorted naïve B cells were treated with αCD40 (1 μg/ml), LPS (2 μg/ml), rIL35 (50 ng/ml) with or without BCL6 and STAT inhibitors; STA-21 (20 μmol/L) for STAT3 (Santa Cruz Biotechnology), Fludarabine (50 μmol/L) for STAT1 (Selleckchem) and 79-6 (100 μmol/L) for BCL6 for 72 hrs. The viability and proliferation of naïve B cells and purified Breg cells treated with STAT1 and STAT3 inhibitors were assessed with MTT (Sigma #M5655) as per manufacturer instructions. After 72 hrs, 10×106 control or BCL6 and STAT inhibited cells were adoptively transferred via tail vein injection into B cell deficient μMT mice. One day after adoptive transfer, 75,000 KPC4662 cells were orthotopically transplanted into the pancreas of μMT mice. Recipient mice were sacrificed 21 days post-tumor cell injections, tumor size and weight were measured, and spleens and tumors were collected for further processing and analysis.

Intracellular cytokine and transcription factor staining was performed as follows. For ex vivo stimulation, sorted cells from tumors or spleens of orthotopic and/or adoptive transfer models (except for B cells, which were cultured in LPS and αCD40 prior to this step) were incubated with PMA (50 ng/mL; Sigma, #P8139) and ionomycin (200 ng/mL; Sigma, #I0634) in the presence of Golgistop Brefeldin A (1X, BioLegend) in complete RPMI medium for 5 hrs at 37 °C. Cells were washed and blocked with αCD16/CD32 (Fc Block, BD Biosciences, 0.1 mg/100,000 cells) for 5 minutes on ice. Viability was assessed using the Live/Dead 7AAD (BioLegend; 420404) stain solution or Live/Dead Aqua cell stain kit (Life Technologies). Cells were then washed and stained with labeled antibodies against surface markers on ice for 30 minutes in FACS buffer (PBS with 3% FCS and 0.05% sodium azide). After surface staining, cells were washed, fixed, and permeabilized using cytofix/cytoperm buffer (BD, 554714) for 15 minutes at 4 °C in the dark. Intracellular staining was performed using fluorophore-conjugated cytokine antibodies for 1 hr at 4 °C in the dark. After intracellular staining, cells were washed and resuspended in FACS buffer for acquisition by flow cytometry. Intracellular staining for Foxp3 was performed using a Foxp3 staining kit (eBioscience, catalog no. 00-5523). Intracellular staining for transcription factors in B cells was performed by using True-Nuclear Transcription Factor Buffer Set (Biolegend; 424401). Briefly, after cell surface staining described above, cells were fixed using True-Nuclear 1× Fix Concentrate at room temperature (RT) in dark for 45 minutes. Cells were washed two times with the True-Nuclear 1× Perm Buffer and a secondary fluorochrome-conjugated antibody diluted in True-Nuclear 1× Perm Buffer was added. Cells were incubated at RT in dark for 1 hr. After incubation, cells were washed 2 times with the True-Nuclear 1× Perm Buffer and resuspended in FACS buffer for acquisition by flow cytometry. For staining of phosphoproteins, cells were fixed with fixation buffer (BioLegend; 420801) at room temperature for 10 minutes and permeabilized with True-Phos perm buffer (BioLegend; 425401) at −20 °C overnight. Cells were then washed twice and resuspended in cell staining buffer (BioLegend; 420201). Fluorophore-conjugated phosphoprotein cocktail antibodies or isotype controls were added and incubated for 60 minutes at 4 °C. After incubation, cells were washed, resuspended in FACS buffer, and samples were acquired on LSR II and LSRII Fortessa (BD Biosciences) and analyzed with FlowJo version 10.2 (TreeStar, Inc.). The human splenic B cells were processed as described above, but the blocking step was done with human BD Fc Block (BD Biosciences, 564219, 0.1 mg/100,000 cells) for 5 minutes on ice.

For ChIP assays, sorted naïve B cells or Breg cells from tumor-bearing or healthy mice were stimulated with αCD40 (1 μg/ml), LPS (2 μg/ml) and rIL35 (50 ng/ml) for 72 hr and 48 hr respectively. After activation and cytokine treatment cells were harvested and cross-linked with 37% (W/V) formaldehyde at final concentration of 1.42% for 15 min at RT. Formaldehyde quenching was done with 125 mM glycine for 5 min at RT. Cell lysis was performed using IP buffer (150 mM NaCl, 50 mM Tris-HCl pH 7.5, 5 mM EDTA, 0.5% V/V NP-40 and 1% V/V Triton X-100). Chromatin was sheared into fragment sizes 500-1000 base pairs in length with four rounds of 15 sec sonication with a 2 min rest between each round using a Diagenode Bioruptor. Sheared chromatin was then subjected to immunoprecipitation with different transcription factor and histone modifier antibodies with isotype matched control antibodies, followed by overnight incubation with rotation. DNA-protein complexes were immune-precipitated with protein A-agarose beads, washed with IP buffer to remove ethanol. Immunoprecipitation with protein A-agarose beads was performed at 4 °C for 1 hr on a rotating platform. The beads were then washed with IP buffer without inhibitors and subjected for DNA isolation. The DNA isolation was performed using 10% (W/V) chelex-100 slurry followed by precipitation of DNA with 70% ethanol. For ChIP-re-ChIP, the DNA-protein complexes were eluted with 0.1 M dithiothreitol followed by a second round of immunoprecipitation with a specific transcription factor antibody, washes with IP buffer and elution with Sodium bicarbonate. DNA was purified using 10% (W/V) chelex-100 slurry followed by precipitation with 70% ethanol. Purified DNA was used to perform real-time PCR with SYBR green master-mix in 10 μl reaction volume (2.5 μl DNA template, 0.3 μl of 10 μM primer pair, 5 μl master-mix and 2.2 μl PCR grade water). Relative occupancy of the immune-precipitated factor at the locus is estimated by using 2\hat{ }(Ctcontrol−Ctsample) equation. Relative enrichment of upstream of transcriptional start site (TSS) is shown and results are scaled to ChIP with control isotype antibody and input. The primers used to perform PCR are listed in Table 1.

For QPCR analysis for gene expression experiments, RNA was extracted from treated cells using the RNeasy Micro Kit (Qiagen). cDNA was generated using High-Capacity cDNA-RT Kit (Invitrogen). QPCR analysis (with 100 ng of DNA template) was performed using the SSO advanced universal SYBR green super-mix reagent (Bio-Rad) and Applied Bio-System platform. Results were normalized to the expression of β-actin, and each sample was run in triplicate. Gene expression was determined by the ΔΔCt method (2-ΔΔCt). Primer sequences are listed in Table 1. β-actin was used to normalize the data by the ΔCt method.

Protein interaction between STAT3 and Pax5 was detected by Duolink proximity ligation assay (PLA; Sigma-Aldrich). Splenic naïve B cells from KPC mice and intratumoral Breg cells from control, BEBi3−/− and Bp35−/− mice were sorted using BD FACS-ARIAIII sorter. Naïve B cells were treated with αCD40/LPS and recombinant cytokines for 72 hrs as indicated above. Breg cells were treated with αCD40/LPS for 48 hrs. After incubation, cells were processed for Duolink proximity ligation assay. Anti-Pax5 (Rabbit) and Anti-pSTAT3 (Mouse) antibodies were conjugated with Duolink In Situ PLA Probe anti-Rabbit PLUS and Duolink In Situ PLA Probe anti-Mouse MINUS (Sigma-Aldrich) respectively. Duolink flow cytometry protocol was followed with few modifications. Briefly, treated naïve B and Breg cells were fixed and permeabilized using BD cytofix/cytoperm buffer (BD Bioscience) followed by blocking with anti-CD16/CD32 (Fc Block, BD Biosciences, 0.1 mg/100,000 cells) for 5 minutes on ice. Samples were incubated with primary anti-rabbit Pax5 PLA-PLUS and anti-mouse pSTAT3 PLA-MINUS antibodies for 1 hr at 37 °C. Ligation, amplification and detection were performed using Duolink flow PLA Detection Kit—Red (Sigma-Aldrich) kit, following manufacturer's instructions. Duolink technical negative control contained only PLA probes but neither Pax5 PLUS nor pSTAT3 MINUS antibodies. The samples were analyzed by LSRII-Fortessa (BD Bioscience) and analyzed by FlowJo version 10.2 (TreeStar, Inc.).

For RNAseq library preparation and analysis, CD19+IgDhiCD1d-CD27−naïve B cells were isolated from spleens of non-tumor bearing and tumor bearing WT and BEBi3−/− mice (two biological replicates per condition) by BD FACS-ARIA III flow cytometry sorting (purity >98%). Sorted naïve B cells were subjected for RNA isolation using the RNeasy Micro Kit (Qiagen). RNAseq Libraries were prepared using the TruSeq Stranded mRNA Library Prep (Illumina, 20020594). In this process, mRNA was isolated using polyA-selection by incubation with poly-T oligo attached magnetic beads. mRNA was then fragmented under elevated temperature with divalent cations. First strand cDNA was generated using reverse transcriptase and random primers with the addition of actinomycin D. Second strand cDNA was generated using DNA Polymerase I with RNase H, and the reaction quenched with the incorporation of dUTP. The 3′ ends were adenylated and dual index adapters ligated using the kit's DNA Ligase enzyme. The final cDNA strands with adapters were amplified to produce the final libraries, which were pooled and diluted to 1.65 pM before being sequenced on a NextSeq500 using the NextSeq 500/550 Mid Output Kit v2.5 (150 Cycles) (Illumina, 20024904). Using the bcl2fastq2 Conversion software 2.20.0 we converted BCL files to FASTQ files and then collapsed the lanes into one file. Total expected read counts were quantified using Salmon 0.9.1(1) using arguments “--gcBias --seqBias”. The UCSC mouse reference genome mm10 used to quantify reads 81. Count data was loaded into R v3.6.3 with tximport v1.12.3, and differential expression analysis was performed using the DESeq2 v1.24.0. Heat maps generated with pheatmap v1.0.12 using the variance stabilized transform (VST). PCA plots generated using both VST data and FactoExtra1.0.7 library. Gene ontogeny analysis was performed using IPA.

Transient luciferase reporter transfection assay was performed in HEK 293T cell line using EBi3, p35 and Cd1d promoter luciferase reporter constructs. The STAT3 and Pax5 binding sites on EBi3, p35 and Cd1d promoter were identified by ChIP. We selected regions −1600 for EBi3, −2200 for p35 and −500 for Cd1d promoters. Mutations in the cloned promoter regions were designed by deleting STAT3-Pax5 binding consensus sequences (FIG. 5I-K). The WT and mutant sequences were cloned into construct containing luciferase reporter and all the WT and mutant vectors were generated by VectorBuilder Inc (Chicago, IL). The WT and mutant constructs were then transfected using Lipofectamine (Sigma-Aldrich) into HEK293T cells. After transfection, the cells were left untreated or treated with IL-6 (20 ng/ml) for 24 hrs. After 24 hrs, cells were processed for luciferase assay using Dual-Luciferase Reporter Assay system (Promega) as per manufacturer instructions and luminescence were measured in single photon counting (SPC) mode on the SpectraMax i3x. Full sequences of EBi3, p35 and Cd1d gene promoter wild-type and mutant constructs will be attached as Supplementary files following acceptance of the manuscript.

For enzyme-linked immunosorbent assays (ELISA), tumor homogenates were prepared by homogenizing tumor tissue with Tissue Extraction Reagent I (ThermoFisher; FNN0071, 50 mM Tris, pH 7.4, 250 mM NaCl, 5 mM EDTA, 2 mM Na3VO4, 1 mM NaF, 20 mM Na4P207, 0.02% NaN3, detergent). The Phosphatase inhibitor cocktail, Protease inhibitor cocktail and PMSF were added just prior to use. Samples were incubated at 4 °C for 1 hr on the orbital shaker and supernatants were collected by centrifuging the tubes at 9000 rpm for 10 min at 4 °C. All samples were stored at −80 °C. The concentration of IgG was measured using mouse IgG ELISA kit (ThermoFisher; 88-50400-22) and concentration of IgM was measured using mouse IgM ELISA kit (ThermoFisher; 88-50470-22) according to manufacturer's instructions.

For Antibody-Dependent Cellular Cytotoxicity (ADCC) experiments, effector cells were peripheral blood mononuclear cells (PBMC) obtained from C57B6/J mice on the same day of the experiment using BD vacutainer tube with sodium heparin (BD Biosciences). For the cytotoxicity assay, effector cells were cultured with target cells (non-cancerous pancreatic cells or tumor cells) at 20:1 (E:T) ratio with and without serum samples from tumor bearing WT, BEBi3−/− and Bp35−/− mice. After incubation for 6 hrs at 37 °C a cell cytotoxicity assays (LDH-Glo Cytotoxicity Assay, J2380 Promega) were performed according to manufacturer's instructions.

For CD8+ T cell depletion studies, 200 μg of anti-CD8 (Bio X Cell, BP0004-1, clone 53-6.7) or an IgG isotype control (Bio X Cell), were administered intra-peritoneally daily starting 3 days prior to tumor cell injection and twice a week after tumor cell injection. In vivo IL35 blockade was described previously 28. Mice were sacrificed 21 days after tumor implantation, tumor size, and weight were measured, and spleen and tumor samples were collected for further processing. Depletion of cells was confirmed by flow cytometry at the end of the experiment.

For immunohistochemistry assays, mouse tumor tissues were fixed in 10% buffered formalin (Fisher Scientific) for 48 hr. Tissues were then washed in 70% ethanol and embedded in paraffin at the Histology Core. Six-micrometer sections were treated with xylenes and rehydrated. Endogenous peroxidase activity was quenched using a solution of 1% hydrogen peroxide (stock of 30% hydrogen peroxide, Sigma) in methanol at room temperature for 10 minutes. Antigen retrieval was done in a microwave oven using 10 mmol/L sodium citrate with 0.05% Tween-20 solution (pH 6.1) for 15 minutes. Blocking was performed for 1 hour at room temperature in a solution of 10% goat serum, 10 mmol/L Tris-HCl, 0.1 mol/L magnesium chloride, 1% BSA, and 0.5% Tween-20. Sections were incubated with primary rat anti-CD19 (Cell Signaling Technology #90176T clone D4V4B) or anti-CD138 (ThermoFisher #36-2900) diluted in 2% BSA/PBS (CD19 1:400 and CD138 1:200) overnight at 4C0. Secondary biotinylated goat anti-rabbit (1:400 final concentration of 3.75 mg/mL) and incubated for 1 hour at room temperature. Tertiary ABC solution was prepared according to the manufacturer's instructions (Vectastain ABC kit, Vector Laboratories) and incubated with slides for 45 minutes at room temperature. Sections were developed using a 3,30-diaminobenzidine tetrahydrochloride kit (DAB peroxidase substrate kit, Vector Laboratories). Slides were then counterstained with Harris hematoxylin (Sigma), dehydrated, and mounted with DPX mounting media (Sigma). Images were acquired using Nikon Eclipse Ni-U microscope with NIS-Elements software (Nikon). CD138+ plasma cells were counted per 20×FOV, counting 3-6 FOV per tumor sample.

For immunofluorescence on human tissues, slides containing fluorescently labeled tissue sections (56 tumor samples and 23 normal adjacent) were scanned in the Aperio ScanScope FL (Leica Biosystems) using the 20× objective and images were archived in TPL's eSlide Manger database (Leica Biosystems). For analysis, expression of CD20, EBi3, CD138 and pan-Cytokeratin was assessed using ImageJ (Fiji). Analysis data included the percentage of CD20, EBi3 co-expressing (Breg) cells and CK-CD138+ expressing plasma cells and the correlation between Breg cells and plasma cells for each antibody marker.

For assays examining human immunoregulatory B cell signature, the B cell signature derived in Mirlekar et al. (2020 Cancer Immunology Research 8:292-308) was used. Briefly, RNA-seq library was prepared from human PBMC conventional and immunoregulatory B-cell populations from healthy volunteers and treatment-naïve PDAC patients. Sequencing was performed on the Illumina HiSeq4000 platform using 150 bp paired-end chemistry and targeting 9×107 reads per sample. FASTQ files were aligned to the human reference genome using STAR v2.4.2. The BAM output files were then quantified using Salmon v0.8.2. FastQC v0.11.7 and MultiQC v1.5 was used to generate quality assurance reports. Statistical analyses were executed in R v3.3.3. Differential gene expression analysis was conducted on the resulting expression matrices using the DESeq2 R package. Genes that were found to be differentially upregulated in tumor-associated Breg subtypes compared with Bcon subtypes, with a Benjamin-Hochberg corrected P value of less than 0.1, were identified. Breg signature was calculated by taking the geometric mean of the expression values of the identified genes.

For quantification and statistical analysis, at least 9 to 21 mice were used in each group, with a minimum of 6 mice in each group per experiment, and the experiments were repeated a 2-3 times to validate reproducibility. Before analysis, data were examined for quality. Group means were compared using Student t-test. Significance in variations between two groups was determined by unpaired Student t-test (two-tailed), experiments with more than two groups used one-way ANOVA comparison; when two groups were tested for more than one condition two-way ANOVA was used. Statistical analysis was performed using GraphPad Prism software. Data are presented as mean±SEM. P<0.05 was considered statistically significant. CIBERSORTx was used to determine the percent of plasma cells in each PAAD TCGA sample (plasma cell signature) (Chen et al. 2018 Methods in Molecular Biology 1711:243). This value was compared to the Breg signature score using Spearman's rank correlation coefficient. Cox proportional hazard model was used to determine the hazard ratio for the plasma cell signature. A T-test was performed to compare the gene expression between conventional B cells and Breg in PAAD samples for select genes. TCGA expression matrices were accessed at firebrowse.org.

TABLE 1
Primers as used in Examples 1 and 2.

| | | | |
|---|---|---|---|
| | SEQ | | SEQ |
| | ID | | ID |
| Forward (5′-3′) | NO | Reverse (5′-3′) | NO |

qPCR (mouse)

| | | | | |
|---|---|---|---|---|
| IL-12a | CATCGATGAGCTG | 1 | CAGATAGCCCATCAC | 2 |
| | ATGCAG | | CCTGT | |
| EBi3 | TGCTCTTCCTGTC | 3 | CGGGATACCGAGAA | 4 |
| | ACTTGCC | | GCATG | |
| CD1d | TCCTAGAGGCAG | 5 | AGCATTTGGCAGGAA | 6 |
| | GGAAGTCA | | ATCAC | |
| STAT3 | AGGAGTCTAACA | 7 | GTGGTACACCTCAGT | 8 |
| | ACGGCAGCCT | | CTCGAAG | |
| Pax5 | AACTTGCCCATCA | 9 | CTGATCTCCCAGGCA | 10 |
| | AGGTGTC | | AACAT | |
| BCL6 | CAGAGATGTGCCT | 11 | CTCCTCAGAGAAACG | 12 |
| | CCATACTGC | | GCAGTCA | |
| IL-10 | CGGGAAGACAAT | 13 | CGGTTAGCAGTATGT | 14 |
| | AACTGCACCC | | TGTCCAGC | |
| Blimp1 | GGCAATCTTGGGA | 15 | GACAGAGGCCGAGTT | 16 |
| | ACTGTGT | | TGAAG | |
| XBP1 | AAGAACACGCTT | 17 | ACTCCCCTTGGCCTC | 18 |
| | GGGAATGG | | CAC | |
| BActin | GGCTGTATTCCCC | 19 | CCAGTTGGTAACAAT | 20 |
| | TCCATCG | | GCCATGT | |

qPCR (Human)

| | | | | |
|---|---|---|---|---|
| Pax5 | CTTGCTCATCAAG | 21 | TGGCGACCTTTGGTT | 22 |
| | GTGTCAGGC | | TGGATCC | |
| BCL6 | CATGCAGAGATGT | 23 | TCAGAGAAGCGGCA | 24 |
| | GCCTCCACA | | GTCACACT | |
| STAT3 | CTTTGAGACCGAG | 25 | GGTCAGCATGTTGTA | 26 |
| | GTGTATCACC | | CCACAGG | |
| PRDM1 | CAGTTCCTAAGAA | 27 | GTGCTGGATTCACAT | 28 |
| | CGCCAACAGG | | AGCGCATC | |
| XBP1 | CTGCCAGAGATCG | 29 | CTCCTGGTTCTCAAC | 30 |
| | AAAGAAGGCGAA | | TACAAGGC | |
| | GGC | | | |
| IL-35(p35) | GATGAGCTGATGC | 31 | AGTCCTCCACCTCGT | 32 |
| | AGGCC | | TGTCCGTGA | |
| IL-35(EBi3) | GCAGACGCCAAC | 33 | CCAGTCACTCAGTTC | 34 |
| | GTCCAC | | CCCGT | |
| IL-10 | GCCTAACATGCTT | 35 | TGATGTCTGGGTCTT | 36 |
| | CGAGATC | | GGTTC | |
| IFN-γ | GAGTGTGGAGAC | 37 | TGCTTTGCGTTGGAC | 38 |
| | CATCAAGGAAG | | ATTCAAGTC | |
| TNF-α | CTCTTCTGCCTGC | 39 | ATGGGCTACAGGCTT | 40 |
| | TGCACTTTG | | GTCACTC | |
| HPRT | CCTGCTGGATTAC | 41 | TCCAACACTTCGTGG | 42 |
| | ATCAAAGCACTG | | GGTCCT | |

Chromatin immunoprecipitation (Mouse)

| | | | | |
|---|---|---|---|---|
| Pax5 promoter- | AGGTTGGACACC | 43 | ACAGGCAAAAATTCT | 44 |
| 1100 | ACACATGTAGAG | | GGACACC | |
| Pax5 promoter- | CCAGGCGCAGGC | 45 | ACTGAGCAGATTTCG | 46 |
| 1600 | AGG | | CCACA | |
| p35 promoter- | CGCACTTGTCCTT | 47 | CTGACCTTGGGAGAC | 48 |
| 250 | GAGATGTAGAG | | ACATTTTTA | |
| p35 promoter- | GCTCTGGACTCAC | 49 | TGTGCTTGCCCAGGA | 50 |
| 1600 | CTGGATGGCAG | | GGTTACAAT | |
| p35 promoter- | TGAGGGACAAAT | 51 | ACCAGACACAGTCCC | 52 |
| 1800 | ATGATCAAAATAC | | TAACCCAT | |
| | AT | | | |
| p35 promoter- | CATGTCATTGCTC | 53 | AGAGGTCAATGCTAT | 54 |
| 2200 | AGGATGGC | | AGGAAAGAAA | |
| EBi3 promoter- | TGGGTGAGATTGT | 55 | AGGAGGGAACTGAG | 56 |
| 250 | AAGTGGTGAGAG | | AGAAACAGAGA | |
| EBi3 promoter- | CCCAGGGTTCTGA | 57 | AGACACCTGTGCAGA | 58 |
| 500 | GCTCTGTTGAT | | ATACCCAAG | |
| EBi3 promoter- | CAGATCCCTCTGC | 59 | CTCCCAGTCATGCAC | 60 |
| 1200 | CCATTC | | TCATC | |
| EBi3 promoter- | TGTGGCCAGATTA | 61 | CCCTTCCATAGTGAG | 62 |
| 1600 | CAGCCTT | | ATATACCC | |
| CD1d promoter- | TCTTGCTTTCGAT | 63 | CTCAGAACCGCTACG | 64 |
| 80 | ATTGAGACGA | | CAAAC | |
| CD1d promoter- | TCCTCACATCAGA | 65 | GCTTTCGTATGCCCA | 66 |
| 500 | CTGACACA | | CAGTT | |
| CD1d promoter- | CAACAAAGATAG | 67 | GCATCGTTTGGGACC | 68 |
| 1300 | CTGCCGCC | | CTACT | |
| BCL6 promoter- | AAAGCGCACTCTC | 69 | CGCGGCTACAAGGCG | 70 |
| 100 | CCTCTTATG | | AC | |
| BCL6 promoter- | GGCTTCACAGAAT | 71 | TTTGCTGAAGCTTTC | 72 |
| 900 | GTTGTAACAGG | | CTGCAC | |
| BCL6 promoter- | ATCAGACAGCTA | 73 | TCCTATCACTGTTGG | 74 |
| 2100 | GCTACACAAAGA | | GAAAATGA | |

Chromatin immunoprecipitation (Human)

| | | | | |
|---|---|---|---|---|
| p35 promoter- | GAGACCTCCACG | 75 | AGCACAGGGCCACTG | 76 |
| 1700 | ACAATGCT | | TTAAA | |
| EBi3 promoter- | CTCTGTCTCCCTC | 77 | GAATTAGGGTGTGGA | 78 |
| 300 | CATGTCC | | GGAGC | |

Genotyping (Mouse)

| | | | | |
|---|---|---|---|---|
| IL-12a (p35) | ACGAGAGTTGCCT | 79 | CCTCATAGATGCTAC | 80 |
| | GGCTACTAG | | CAAGGCAC | |
| EBi3 | CTCTCAAGTACCG | 81 | CTGAGCTGACACCTG | 82 |
| | ACTCCGCTA | | GATGCAA | |
| βActin | GGCTGTATTCCCC | 83 | CCAGTTGGTAACAAT | 84 |
| | TCCATCG | | GCCATGT | |

The foregoing is illustrative of the present invention, and is not to be construed as limiting thereof. The invention is defined by the following claims, with equivalents of the claims to be included therein.

1. A method of producing a reprogrammed B cell, comprising
culturing a B cell in a medium with a reprogramming factor to produce a reprogrammed B cell.

2-5. (canceled)

6. A method of treating a cancer in a subject, comprising the steps of:
(a) culturing a B cell in a medium with a reprogramming factor to produce a population of one or more reprogrammed B cells; and
(b) delivering the reprogrammed B cells to the subject,
wherein the reprogrammed B cells comprise anti-tumor functions, thereby treating the cancer in the subject.

7. A method of enhancing an immune response to cancer in a subject, comprising the steps of:
(a) culturing a B cell in a medium with a reprogramming factor to produce a population of one or more reprogrammed B cells; and
(b) delivering the reprogrammed B cells to the subject,
wherein the reprogrammed B cells comprise anti-tumor functions,
thereby the immune response to the cancer in the subject.

8. The method of claim 6, wherein the anti-tumor functions comprise enhanced production of immunosuppressive cytokines, enhanced T cell interaction and/or activation, enhanced plasma B cell differentiation, reduced B regulatory cell (“Breg”) differentiation, reduced IL10 expression, reduced IL35 expression, and/or reduced TGFβ expression.

9. The method of claim 6, wherein the reprogrammed B cells are and/or differentiate into plasma B cells.

10. The method of claim 6, wherein the reprogramming factor comprises a STAT3 pathway activator and/or inhibitor.

11. (canceled)

12. The method of claim 6, wherein the culturing comprises incubating the B cell with the reprogramming factor for about 30 min to about 96 hours.

13. The method of claim 6 wherein the culturing comprises incubating the B cell with the reprogramming factor in a concentration of about 1 μmol/L to about 1000 μmol/L.

14. The method of claim 6, wherein the B cell is a liver-sourced B cell, a blood-circulatory-sourced B cell, a B cell sourced from a cancer proximal environment, a B cell sourced from a cancer nonproximal environment, or any combination thereof.

15-17. (canceled)

18. The method of claim 6, wherein the subject has, is suspected to have, and/or is at risk of cancer.

19. The method of claim 18, wherein the cancer is a solid tumor.

20. The method of claim 18, wherein the cancer is a liquid cancer.

21. The method of claim 6, further comprising a step of isolating the B cell from the subject prior to ex vivo culturing.

22. The method of claim 6, wherein delivering the reprogrammed B cells comprises at least one or more iterative administrations of the reprogrammed B cell.

23. The method of claim 6, further comprising co-delivering a therapeutic agent to the subject.

24. The method of claim 23, wherein co-delivering comprises concurrent delivery, delivering the therapeutic agent prior to delivery of the reprogrammed B cells, delivering the therapeutic agent after delivery of the reprogrammed B cells, or any combination thereof.

25-26. (canceled)

27. The method of claim 23, wherein the therapeutic agent is an immunotherapy agent.

28. (canceled)

29. A composition comprising a reprogrammed B cell for use in the method of claim 6.

30. The composition of claim 29, further comprising a pharmaceutical carrier, diluent, and/or adjuvant.

31. A kit comprising the composition of claim 29, and optional instructions for the use thereof.


SEQUENCE LISTING (84 sequences)

SEQ ID NO: 1 - DNA, 19 bp, synthetic construct
  source 1..19: mol_type = other DNA; note = IL12a forward primer
catcgatgagctgatgcag

SEQ ID NO: 2 - DNA, 20 bp, synthetic construct
  source 1..20: mol_type = other DNA; note = IL12a reverse primer
cagatagcccatcaccctgt

SEQ ID NO: 3 - DNA, 20 bp, synthetic construct
  source 1..20: mol_type = other DNA; note = EBi3 forward primer
tgctcttcctgtcacttgcc

SEQ ID NO: 4 - DNA, 19 bp, synthetic construct
  source 1..19: mol_type = other DNA; note = CGGGATACCGAGAAGCATG
cgggataccgagaagcatg

SEQ ID NO: 5 - DNA, 20 bp, synthetic construct
  source 1..20: mol_type = other DNA; note = CD1d forward primer
tcctagaggcagggaagtca

SEQ ID NO: 6 - DNA, 20 bp, synthetic construct
  source 1..20: mol_type = other DNA; note = CD1d reverse primer
agcatttggcaggaaatcac

SEQ ID NO: 7 - DNA, 22 bp, synthetic construct
  source 1..22: mol_type = other DNA; note = STAT3 forward primer
aggagtctaacaacggcagcct

SEQ ID NO: 8 - DNA, 22 bp, synthetic construct
  source 1..22: mol_type = other DNA; note = STAT3 reverse primer
gtggtacacctcagtctcgaag

SEQ ID NO: 9 - DNA, 20 bp, synthetic construct
  source 1..20: mol_type = other DNA; note = Pax5 forward primer
aacttgcccatcaaggtgtc

SEQ ID NO: 10 - DNA, 20 bp, synthetic construct
  source 1..20: mol_type = other DNA; note = Pax5 reverse primer
ctgatctcccaggcaaacat

SEQ ID NO: 11 - DNA, 22 bp, synthetic construct
  source 1..22: mol_type = other DNA; note = BCL6 forward primer
cagagatgtgcctccatactgc

SEQ ID NO: 12 - DNA, 22 bp, synthetic construct
  source 1..22: mol_type = other DNA; note = BCL6 reverse primer
ctcctcagagaaacggcagtca

SEQ ID NO: 13 - DNA, 22 bp, synthetic construct
  source 1..22: mol_type = other DNA; note = IL10 forward primer
cgggaagacaataactgcaccc

SEQ ID NO: 14 - DNA, 23 bp, synthetic construct
  source 1..23: mol_type = other DNA; note = IL10 reverse primer
cggttagcagtatgttgtccagc

SEQ ID NO: 15 - DNA, 20 bp, synthetic construct
  source 1..20: mol_type = other DNA; note = Blimp1 forward primer
ggcaatcttgggaactgtgt

SEQ ID NO: 16 - DNA, 20 bp, synthetic construct
  source 1..20: mol_type = other DNA; note = Blimp1 reverse primer
gacagaggccgagtttgaag

SEQ ID NO: 17 - DNA, 20 bp, synthetic construct
  source 1..20: mol_type = other DNA; note = XBP1 forward primer
aagaacacgcttgggaatgg

SEQ ID NO: 18 - DNA, 18 bp, synthetic construct
  source 1..18: mol_type = other DNA; note = XBP1 reverse primer
actccccttggcctccac

SEQ ID NO: 19 - DNA, 20 bp, synthetic construct
  source 1..20: mol_type = other DNA; note = beta-actin forward primer
ggctgtattcccctccatcg

SEQ ID NO: 20 - DNA, 22 bp, synthetic construct
  source 1..22: mol_type = other DNA; note = beta-actin reverse primer
ccagttggtaacaatgccatgt

SEQ ID NO: 21 - DNA, 22 bp, synthetic construct
  source 1..22: mol_type = other DNA; note = pax5 forward primer human
cttgctcatcaaggtgtcaggc

SEQ ID NO: 22 - DNA, 22 bp, synthetic construct
  source 1..22: mol_type = other DNA; note = pax5 reverse primer human
tggcgacctttggtttggatcc

SEQ ID NO: 23 - DNA, 22 bp, synthetic construct
  source 1..22: mol_type = other DNA; note = BCL6 forward primer human
catgcagagatgtgcctccaca

SEQ ID NO: 24 - DNA, 22 bp, synthetic construct
  source 1..22: mol_type = other DNA; note = BCL6 reverse primer human
tcagagaagcggcagtcacact

SEQ ID NO: 25 - DNA, 23 bp, synthetic construct
  source 1..23: mol_type = other DNA; note = STAT3 forward primer human
ctttgagaccgaggtgtatcacc

SEQ ID NO: 26 - DNA, 22 bp, synthetic construct
  source 1..22: mol_type = other DNA; note = STAT3 reverse primer human
ggtcagcatgttgtaccacagg

SEQ ID NO: 27 - DNA, 23 bp, synthetic construct
  source 1..23: mol_type = other DNA; note = PRDM1 forward primer human
cagttcctaagaacgccaacagg

SEQ ID NO: 28 - DNA, 23 bp, synthetic construct
  source 1..23: mol_type = other DNA; note = PRDM1 reverse primer human
gtgctggattcacatagcgcatc

SEQ ID NO: 29 - DNA, 28 bp, synthetic construct
  source 1..28: mol_type = other DNA; note = XBP1 forward primer human
ctgccagagatcgaaagaaggcgaaggc

SEQ ID NO: 30 - DNA, 23 bp, synthetic construct
  source 1..23: mol_type = other DNA; note = XBP1 reverse primer human
ctcctggttctcaactacaaggc

SEQ ID NO: 31 - DNA, 18 bp, synthetic construct
  source 1..18: mol_type = other DNA; note = il35p35 forward primer human
gatgagctgatgcaggcc

SEQ ID NO: 32 - DNA, 24 bp, synthetic construct
  source 1..24: mol_type = other DNA; note = il35p35 reverse primer human
agtcctccacctcgttgtccgtga

SEQ ID NO: 33 - DNA, 18 bp, synthetic construct
  source 1..18: mol_type = other DNA; note = il35EBi3 forward primer human
gcagacgccaacgtccac

SEQ ID NO: 34 - DNA, 20 bp, synthetic construct
  source 1..20: mol_type = other DNA; note = il35EBi3 reverse primer human
ccagtcactcagttccccgt

SEQ ID NO: 35 - DNA, 20 bp, synthetic construct
  source 1..20: mol_type = other DNA; note = IL10 forward primer human
gcctaacatgcttcgagatc

SEQ ID NO: 36 - DNA, 20 bp, synthetic construct
  source 1..20: mol_type = other DNA; note = IL10 reverse primer human
tgatgtctgggtcttggttc

SEQ ID NO: 37 - DNA, 23 bp, synthetic construct
  source 1..23: mol_type = other DNA; note = IFNgamma forward primer human
gagtgtggagaccatcaaggaag

SEQ ID NO: 38 - DNA, 24 bp, synthetic construct
  source 1..24: mol_type = other DNA; note = IFNgamma reverse primer human
tgctttgcgttggacattcaagtc

SEQ ID NO: 39 - DNA, 22 bp, synthetic construct
  source 1..22: mol_type = other DNA; note = TNFalpha forward primer human
ctcttctgcctgctgcactttg

SEQ ID NO: 40 - DNA, 22 bp, synthetic construct
  source 1..22: mol_type = other DNA; note = TNFalpha reverse primer human
atgggctacaggcttgtcactc

SEQ ID NO: 41 - DNA, 25 bp, synthetic construct
  source 1..25: mol_type = other DNA; note = HPRT forward primer human
cctgctggattacatcaaagcactg

SEQ ID NO: 42 - DNA, 21 bp, synthetic construct
  source 1..21: mol_type = other DNA; note = HPRT reverse primer human
tccaacacttcgtggggtcct

SEQ ID NO: 43 - DNA, 24 bp, synthetic construct
  source 1..24: mol_type = other DNA; note = mouse Pax5 promoter -1100 forward
aggttggacaccacacatgtagag

SEQ ID NO: 44 - DNA, 22 bp, synthetic construct
  source 1..22: mol_type = other DNA; note = mouse Pax5 promoter -1100 reverse
acaggcaaaaattctggacacc

SEQ ID NO: 45 - DNA, 15 bp, synthetic construct
  source 1..15: mol_type = other DNA; note = mouse Pax5 promoter -1600 forward
ccaggcgcaggcagg

SEQ ID NO: 46 - DNA, 20 bp, synthetic construct
  source 1..20: mol_type = other DNA; note = mouse Pax5 promoter -1600 reverse
actgagcagatttcgccaca

SEQ ID NO: 47 - DNA, 24 bp, synthetic construct
  source 1..24: mol_type = other DNA; note = mouse p35 promoter -250 forward
cgcacttgtccttgagatgtagag

SEQ ID NO: 48 - DNA, 24 bp, synthetic construct
  source 1..24: mol_type = other DNA; note = mouse p35 promoter -250 reverse
ctgaccttgggagacacattttta

SEQ ID NO: 49 - DNA, 24 bp, synthetic construct
  source 1..24: mol_type = other DNA; note = mouse p35 promoter -1600 forward
gctctggactcacctggatggcag

SEQ ID NO: 50 - DNA, 24 bp, synthetic construct
  source 1..24: mol_type = other DNA; note = mouse p35 promoter -1600 reverse
tgtgcttgcccaggaggttacaat

SEQ ID NO: 51 - DNA, 27 bp, synthetic construct
  source 1..27: mol_type = other DNA; note = mouse p35 promoter -1800 forward
tgagggacaaatatgatcaaaatacat

SEQ ID NO: 52 - DNA, 23 bp, synthetic construct
  source 1..23: mol_type = other DNA; note = mouse p35 promoter -1800 reverse
accagacacagtccctaacccat

SEQ ID NO: 53 - DNA, 21 bp, synthetic construct
  source 1..21: mol_type = other DNA; note = mouse p35 promoter -2200 forward
catgtcattgctcaggatggc

SEQ ID NO: 54 - DNA, 25 bp, synthetic construct
  source 1..25: mol_type = other DNA; note = mouse p35 promoter -2200 reverse
agaggtcaatgctataggaaagaaa

SEQ ID NO: 55 - DNA, 25 bp, synthetic construct
  source 1..25: mol_type = other DNA; note = mouse EBi3 promoter -250 forward
tgggtgagattgtaagtggtgagag

SEQ ID NO: 56 - DNA, 25 bp, synthetic construct
  source 1..25: mol_type = other DNA; note = mouse EBi3 promoter -250 reverse
aggagggaactgagagaaacagaga

SEQ ID NO: 57 - DNA, 24 bp, synthetic construct
  source 1..24: mol_type = other DNA; note = mouse EBi3 promoter -500 forward
cccagggttctgagctctgttgat

SEQ ID NO: 58 - DNA, 24 bp, synthetic construct
  source 1..24: mol_type = other DNA; note = mouse EBi3 promoter -500 reverse
agacacctgtgcagaatacccaag

SEQ ID NO: 59 - DNA, 19 bp, synthetic construct
  source 1..19: mol_type = other DNA; note = mouse EBi3 promoter -1200 forward
cagatccctctgcccattc

SEQ ID NO: 60 - DNA, 20 bp, synthetic construct
  source 1..20: mol_type = other DNA; note = mouse EBi3 promoter -1200 reverse
ctcccagtcatgcactcatc

SEQ ID NO: 61 - DNA, 20 bp, synthetic construct
  source 1..20: mol_type = other DNA; note = mouse EBi3 promoter -1600 forward
tgtggccagattacagcctt

SEQ ID NO: 62 - DNA, 23 bp, synthetic construct
  source 1..23: mol_type = other DNA; note = mouse EBi3 promoter -1600 reverse
cccttccatagtgagatataccc

SEQ ID NO: 63 - DNA, 23 bp, synthetic construct
  source 1..23: mol_type = other DNA; note = mouse CD1d promoter -80 forward
tcttgctttcgatattgagacga

SEQ ID NO: 64 - DNA, 20 bp, synthetic construct
  source 1..20: mol_type = other DNA; note = mouse CD1d promoter -80 reverse
ctcagaaccgctacgcaaac

SEQ ID NO: 65 - DNA, 21 bp, synthetic construct
  source 1..21: mol_type = other DNA; note = mouse CD1d promoter -500 forward
tcctcacatcagactgacaca

SEQ ID NO: 66 - DNA, 20 bp, synthetic construct
  source 1..20: mol_type = other DNA; note = mouse CD1d promoter -500 reverse
gctttcgtatgcccacagtt

SEQ ID NO: 67 - DNA, 20 bp, synthetic construct
  source 1..20: mol_type = other DNA; note = mouse CD1d promoter -1300 forward
caacaaagatagctgccgcc

SEQ ID NO: 68 - DNA, 20 bp, synthetic construct
  source 1..20: mol_type = other DNA; note = mouse CD1d promoter -1300 reverse
gcatcgtttgggaccctact

SEQ ID NO: 69 - DNA, 22 bp, synthetic construct
  source 1..22: mol_type = other DNA; note = mouse BCL6 promoter -100 forward
aaagcgcactctccctcttatg

SEQ ID NO: 70 - DNA, 17 bp, synthetic construct
  source 1..17: mol_type = other DNA; note = mouse BCL6 promoter -100 reverse
cgcggctacaaggcgac

SEQ ID NO: 71 - DNA, 24 bp, synthetic construct
  source 1..24: mol_type = other DNA; note = mouse BCL6 promoter -900 forward
ggcttcacagaatgttgtaacagg

SEQ ID NO: 72 - DNA, 21 bp, synthetic construct
  source 1..21: mol_type = other DNA; note = mouse BCL6 promoter -900 reverse
tttgctgaagctttcctgcac

SEQ ID NO: 73 - DNA, 24 bp, synthetic construct
  source 1..24: mol_type = other DNA; note = mouse BCL6 promoter -2100 forward
atcagacagctagctacacaaaga

SEQ ID NO: 74 - DNA, 23 bp, synthetic construct
  source 1..23: mol_type = other DNA; note = mouse BCL6 promoter -2100 reverse
tcctatcactgttgggaaaatga

SEQ ID NO: 75 - DNA, 20 bp, synthetic construct
  source 1..20: mol_type = other DNA; note = human p35 promoter -1700 forward
gagacctccacgacaatgct

SEQ ID NO: 76 - DNA, 20 bp, synthetic construct
  source 1..20: mol_type = other DNA; note = human p35 promoter -1700 reverse
agcacagggccactgttaaa

SEQ ID NO: 77 - DNA, 20 bp, synthetic construct
  source 1..20: mol_type = other DNA; note = human EBi3 promoter -300 forward
ctctgtctccctccatgtcc

SEQ ID NO: 78 - DNA, 20 bp, synthetic construct
  source 1..20: mol_type = other DNA; note = human EBi3 promoter -300 reverse
gaattagggtgtggaggagc

SEQ ID NO: 79 - DNA, 22 bp, synthetic construct
  source 1..22: mol_type = other DNA; note = mouse IL12ap35 genotyping forward
acgagagttgcctggctactag

SEQ ID NO: 80 - DNA, 23 bp, synthetic construct
  source 1..23: mol_type = other DNA; note = mouse IL12ap35 genotyping reverse
cctcatagatgctaccaaggcac

SEQ ID NO: 81 - DNA, 22 bp, synthetic construct
  source 1..22: mol_type = other DNA; note = mouse EBi3 genotyping forward
ctctcaagtaccgactccgcta

SEQ ID NO: 82 - DNA, 22 bp, synthetic construct
  source 1..22: mol_type = other DNA; note = mouse EBi3 genotyping reverse
ctgagctgacacctggatgcaa

SEQ ID NO: 83 - DNA, 20 bp, synthetic construct
  source 1..20: mol_type = other DNA; note = mouse betaActin genotyping forward
ggctgtattcccctccatcg

SEQ ID NO: 84 - DNA, 22 bp, synthetic construct
  source 1..22: mol_type = other DNA; note = mouse betaActin genotyping reverse
ccagttggtaacaatgccatgt